(19) United States
(12) Patent Application Publication
(10) Pub. No.: US 2026/0234111 A1
(43) Pub. Date: Aug. 13, 2026

(54) ISOTOPOLOGUES OF AMINOHETEROARYL KINASE INHIBITORS AND USES THEREOF

(71) Applicant: Allorion Therapeutics Inc, Natick, MA (US)

(72) Inventors: Dai CHENG, Guangzhou (CN); Xiaohang YIN, Guangzhou (CN); Zeqiang XIE, Guangzhou (CN)

(21) Appl. No.: 19/431,098

(22) Filed: Dec. 23, 2025

Related U.S. Application Data
(63) Continuation of application No. PCT/US2024/035290, filed on Jun. 24, 2024.

(30) Foreign Application Priority Data
Jun. 25, 2023 (WO) PCT/CN2023/102149

(51) Int. Cl. C07D 239/30; C07B 59/00; C07C 303/40; C07C 311/16; C07D 309/10

(52) U.S. Cl. CPC C07D 239/30; C07B 59/001; C07B 59/002; C07C 303/40; C07C 311/16; C07D 309/10; C07B 2200/05

(57) ABSTRACT

Provided are isotopologues of aminoheteroaryl compounds for inhibiting cyclin-dependent kinases, processes for the preparation of the compounds, pharmaceutical compositions comprising the compounds, and methods of treatment of diseases and disorders using the compounds or pharmaceutical compositions.

### CROSS-REFERENCE TO RELATED APPLICATIONS

This application is a continuation of International Application No. PCT/US2024/035290, filed Jun. 24, 2024, which claims the benefit of International Patent Application No. PCT/CN2023/102149, filed Jun. 25, 2023, the disclosures of which are hereby incorporated by reference in their entirety.

### BACKGROUND

Cyclin-dependent kinase (CDKs) are a family of serine/threonine protein kinases that regulate the cell cycle progression. Among CDKs, CDK2 is an essential driver for cells to transition from late G1 into S and G2 phases. During late G1, CDK2 is activated upon binding to cyclin E. The cyclin E/CDK2 complex hyper-phosphorylates RB to release E2F from Rb and initiate transcription of genes necessary for G1/S transition. Subsequently, CDK2 forms complex with Cyclin A to regulate S phase progression by activating proteins important for DNA replication and centrosome duplication, such as DNA replication licensing protein (CDC6) and centrosome protein CP110 (Tadesse et al. Targeting CDK2 in cancer: challenges and opportunities for therapy, Drug Discovery Today. 2019; 25(2): 406-413).

Cyclin E1 is frequently amplified and/or overexpressed in human cancer. In high grade serous ovarian cancer, cyclin E1 amplification is detected in approximately 20% of patients and is associated with chemo resistance/refractory (TCGA, Integrated genomic analyses of ovarian carcinoma, Nature. 2011; 474: 609-615; Nakayama et al; Gene amplification CCNE1 is related to poor survival and potential therapeutic target in ovarian cancer, Cancer (2010) 116: 2621-34). Cyclin E1 amplified ovarian cancer cell lines are sensitive to reagents that either inhibit CDK2 activity or decrease cellular CDK2 protein level, suggesting CDK2 dependence in these cyclin E1 amplified cells (Au-Yeung et al. Selective targeting of cyclin E1 amplified high grade serous ovarian cancer by clin-dependent kinase 2 and AKT inhibition, Clin. Cancer Res. 2017; 23(7):1862-1874). Poor outcomes and drug resistance were also associated with high Cyclin E1 expression in endometrial, gastric, breast and other cancers (Noske et al., Detection of CCNE1/URI (19q12) amplification by in situ hybridization is common in high grade and type II endometrial cancer, Oncotarget (2017) 8: 14794-14805; Ooi et al., Gene amplification of CCNE1, CCND1 and CDK6 in gastric cancers detected by multiplex ligation-dependent probe amplification and fluorescence in situ hybridization, Hum Pathol. (2017) 61:58-67; Keyomarsi et al., Cyclin E and survival in patients with breast cancer. N Engl J Med. (2002) 347: 1566-75). Estrogen receptor (ER) positive breast cancer cell lines with acquired resistance to CDK4/6 inhibitor Palbociclib has elevated cyclin E1 expression and can be re-sensitized upon knock down of CDK2 (Herrera-Abreu et al., Early adaptation and acquired resistance to CDK4/6 inhibition in estrogen receptor-positive breast cancer, Cancer Res. (2016) 76: 2301-2313). High cyclin E1 level was also reported to associate with poor response to Palbociclib plus fulvestrant combo therapy in ER+BC (CCNE1 high vs CCNE1 low: median PFS for Palbociclib+fulvestrant arm, 7.6 v 14.1 month; placebo+fulvestrant arm, 4.0 v 4.8 month) further underline the importance of CDK2 activity in mediating resistance to CDK4/6 inhibitors (Turner et al., Cyclin E1 expression and Palbociclib efficacy in previously treated hormone receptor positive metastatic breast cancer Clin Oncol. (2019) 37(14): 1169-1178).

Cyclin E2 (CCNE2) overexpression was reported as associated with endocrine resistance in breast cancer cells and CDK2 inhibition has been reported to restore sensitivity to tamoxifen or CDK4 inhibitors in tamoxifen-resistant and CCNE2 overexpressing cells. (Caldon et al., Cyclin E2 overexpression is associated with endocrine resistance but not insensitivity to CDK2 inhibition in human breast cancer cells. Mol Cancer Ther. (2012) 11:1488-99; Herrera-Abreu et al., Early Adaptation and Acquired Resistance to CDK4/6 Inhibition in Estrogen Receptor-Positive Breast Cancer, Cancer Res. (2016) 76: 2301-2313). Additionally, Cyclin E amplification has also been reported as contributing to trastuzumab resistance in HER2+ breast cancer. (Scaltriti et al. Cyclin E amplification/overexpression is a mechanism of trastuzumab resistance in HER2+ breast cancer patients, Proc Natl Acad Sci. (2011) 108: 3761-6). Further, Cyclin E overexpression was reported to play a role in basal-like and triple negative breast cancer (TNBC), as well as inflammatory breast cancer. (Elsawaf & Sinn, Triple Negative Breast Cancer: Clinical and Histological Correlations, Breast Care (2011) 6:273-278; Alexander et al., Cyclin E overexpression as a biomarker for combination treatment strategies in inflammatory breast cancer, Oncotarget (2017) 8: 14897-14911.)

### SUMMARY

Provided herein are compounds of Formula (I):

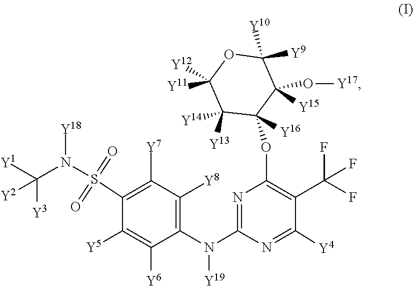

or a stereoisomer, a mixture of stereoisomers, tautomer, or a pharmaceutically acceptable salt thereof, wherein at least one of Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, Y¹⁶, Y¹⁷, Y¹⁸, and Y¹⁹ is a hydrogen that is isotopically enriched with deuterium, and the others of Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, Y¹⁶, Y¹⁷, Y¹⁸, and Y¹⁹ are non-enriched hydrogen atoms.

Also provided herein are pharmaceutical compositions comprising a compound described herein, such as compounds of Formula (I) and any sub-formula.

Also provided herein are methods of treating disease or disorders, such as cancer, using a compound described herein, such as a compound of Formula (I) and any sub-formula.

### DETAILED DESCRIPTION

### Definitions

To facilitate understanding of the application set forth herein, a number of terms are defined below. Generally, the nomenclature used herein and the laboratory procedures in organic chemistry, medicinal chemistry, and pharmacology described herein are those well known and commonly employed in the art. Unless defined otherwise, all technical and scientific terms used herein generally have the same meaning as commonly understood by one of ordinary skill in the art to which this application belongs.

The term “isotopic composition” refers to the amount of each isotope present for a given atom, and “natural isotopic composition” refers to the naturally occurring isotopic composition or abundance for a given atom. Atoms containing their natural isotopic composition may also be referred to herein as “non-enriched” atoms. Unless otherwise designated, the atoms of the compounds recited herein are meant to represent any stable isotope of that atom. For example, unless otherwise stated, when a position is designated specifically as “H” or “hydrogen,” the position is understood to have hydrogen at its natural isotopic composition.

The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. As used herein, an “isotopologue” is an isotopically enriched compound.

The term “isotopic enrichment” refers to a process by which a given atom or compound is isotopically enriched.

The term “isotopic enrichment factor” refers to the ratio between the isotopic composition and the natural isotopic composition of a specified isotope.

With regard to the compounds provided herein, when a particular atomic position is designated as deuterium or “D,” it is understood that the abundance of deuterium at that position is substantially greater than the natural abundance of deuterium, which is about 0.0156%. A position designated as deuterium typically has a minimum isotopic enrichment factor of, in certain embodiments, at least about 1000 (about 15% deuterium incorporation), at least about 2000 (about 30% deuterium incorporation), at least about 3000 (about 45% deuterium incorporation), at least about 3500 (about 52.5% deuterium incorporation), at least about 4000 (about 60% deuterium incorporation), at least about 4500 (about 67.5% deuterium incorporation), at least about 5000 (about 75% deuterium incorporation), at least about 5500 (about 82.5% deuterium incorporation), at least about 6000 (about 90% deuterium incorporation), at least about 6333.3 (about 95% deuterium incorporation), at least about 6466.7 (about 97% deuterium incorporation), at least about 6600 (about 99% deuterium incorporation), or at least about 6633.3 (about 99.5% deuterium incorporation) at each designated deuterium atom.

The isotopic enrichment and isotopic enrichment factor of the compounds provided herein can be determined using conventional analytical methods known to one of ordinary skill in the art, including mass spectrometry and nuclear magnetic resonance spectroscopy.

Definitions of specific functional groups and chemical terms are described in more detail below. The chemical elements are identified in accordance with the Periodic Table of the Elements, CAS version, Handbook of Chemistry and Physics, 75th ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5th ed., John Wiley & Sons, Inc., New York, 2001; Larock, Comprehensive Organic Transformations, VCH Publishers, Inc., New York, 1989; and Carruthers, Some Modern Methods of Organic Synthesis, 3rd ed., Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high performance liquid chromatography (HPLC), chiral supercritical fluid chromatograph (SFC), and the formation and crystallization of chiral salts; or isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The application additionally encompasses compounds described herein as individual isomers substantially free of other isomers, and alternatively, as mixtures of various isomers including racemic mixtures. When a stereochemistry is specifically drawn, unless otherwise contradictory from context, it should be understood that with respect to that particular chiral center or axial chirality, the compound can exist predominantly as the as-drawn stereoisomer, such as with less than 20%, less than 10%, less than 5%, less than 1%, by weight, by HPLC or SFC area, or both, or with a non-detectable amount of the other stereoisomer(s). For example, in some embodiments, the compound can exist predominantly as the as-drawn stereoisomer having an enantiomeric excess (“ee”) of greater than 80%, such as having an ee of 90% or above, 95% or above, 98% or above, 99% or above, or have a non-detectable amount of the other enantiomer. The presence and/or amounts of stereoisomers can be determined by those skilled in the art in view of the present application, including through the use of a chiral HPLC or chiral SFC. As understood by those skilled in the art, when a “*” is shown in the chemical structures herein, unless otherwise contradictory from context, it is to designate that the corresponding chiral center is enantiomerically pure or enriched in either of the configurations or is enantiomerically pure or enriched in the as-dawn configuration, such as with less than 20%, less than 10%, less than 5%, less than 1%, by weight, by HPLC or SFC area, or both, or with a non-detectable amount of the other stereoisomer(s). Also, when no stereochemistry is specifically drawn, and no “*” is used in the chemical structures, unless otherwise contradictory from context, it should be understood that such structures include the corresponding compound in any stereoisomeric forms, including individual isomers substantially free of other isomers and mixtures of various isomers including racemic mixtures.

In some embodiments, the compound is a racemic mixture of (S)- and (R)-isomers. In other embodiments, provided herein is a mixture of compounds wherein individual compounds of the mixture exist predominately in an (S)- or (R)-isomeric configuration. For example, in some embodiments, the compound mixture has an (S)-enantiomeric excess of greater than about 10%, greater than about 20%, greater than about 30%, greater than about 40%, greater than about 50%, greater than about 55%, greater than about 60%, greater than about 65%, greater than about 70%, greater than about 75%, greater than about 80%, greater than about 85%, greater than about 90%, greater than about 95%, greater than about 96%, greater than about 97%, greater than about 98%, or greater than about 99%. In some embodiments, the compound mixture has an (S)-enantiomeric excess of about 55%, about 60%, about 65%, about 70%, about 75%, about 80%, about 85%, about 90%, about 95%, about 96%, about 97%, about 98%, about 99%, or about 99.5%, or more. In some embodiments, the compound mixture has an (S)-enantiomeric excess of about 55% to about 99.5%, about 60% to about 99.5%, about 65% to about 99.5%, about 70% to about 99.5%, about 75% to about 99.5%, about 80% to about 99.5%, about 85% to about 99.5%, about 90% to about 99.5%, about 95% to about 99.5%, about 96% to about 99.5%, about 97% to about 99.5%, about 98% to about 99.5%, or about 99% to about 99.5%, or more than about 99.5%.

In other embodiments, the compound mixture has an (R)-enantiomeric excess of greater than about 10%, greater than about 20%, greater than about 30%, greater than about 40%, greater than about 50%, greater than about 55%, greater than about 60%, greater than about 65%, greater than about 70%, greater than about 75%, greater than about 80%, greater than about 85%, greater than about 90%, greater than about 95%, greater than about 96%, greater than about 97%, greater than about 98%, or greater than about 99%. In some embodiments, the compound mixture has an (R)-enantiomeric excess of about 55%, about 60%, about 65%, about 70%, about 75%, about 80%, about 85%, about 90%, about 95%, about 96%, about 97%, about 98%, about 99%, or about 99.5%, or more. In some embodiments, the compound mixture has an (R)-enantiomeric excess of about 55% to about 99.5%, about 60% to about 99.5%, about 65% to about 99.5%, about 70% to about 99.5%, about 75% to about 99.5%, about 80% to about 99.5%, about 85% to about 99.5%, about 90% to about 99.5%, about 95% to about 99.5%, about 96% to about 99.5%, about 97% to about 99.5%, about 98% to about 99.5%, or about 99% to about 99.5%, or more than about 99.5%.

In other embodiments, the compound mixture contains identical chemical entities except for their stereochemical orientations, namely (S)- or (R)-isomers. For example, if a compound provided herein has —CH(R)— unit, and R is not hydrogen, then the —CH(R)— is in an (S)- or (R)-stereochemical orientation for each of the identical chemical entities (i.e., (S)- or (R)-stereoisomers). In some embodiments, the mixture of identical chemical entities (i.e., mixture of stereoisomers) is a racemic mixture of (S)- and (R)-isomers. In other embodiments, the mixture of the identical chemical entities (i.e., mixture of stereoisomers) contains predominately (S)-isomer or predominately (R)-isomer. For example, in some embodiments, the (S)-isomer in the mixture of identical chemical entities (i.e., mixture of stereoisomers) is present at about 55%, about 60%, about 65%, about 70%, about 75%, about 80%, about 85%, about 90%, about 95%, about 96%, about 97%, about 98%, about 99%, or about 99.5% by weight, or more, relative to the total weight of the mixture of (S)- and (R)-isomers. In some embodiments, the (S)-isomer in the mixture of identical chemical entities (i.e., mixture of stereoisomers) is present at an (S)-enantiomeric excess of about 10% to about 99.5%, about 20% to about 99.5%, about 30% to about 99.5%, about 40% to about 99.5%, about 50% to about 99.5%, about 55% to about 99.5%, about 60% to about 99.5%, about 65% to about 99.5%, about 70% to about 99.5%, about 75% to about 99.5%, about 80% to about 99.5%, about 85% to about 99.5%, about 90% to about 99.5%, about 95% to about 99.5%, about 96% to about 99.5%, about 97% to about 99.5%, about 98% to about 99.5%, or about 99% to about 99.5%, or more than about 99.5%.

In other embodiments, the (R)-isomer in the mixture of identical chemical entities (i.e., mixture of stereoisomers) is present at about 55%, about 60%, about 65%, about 70%, about 75%, about 80%, about 85%, about 90%, about 95%, about 96%, about 97%, about 98%, about 99%, or about 99.5% by weight, or more, relative to the total weight of the mixture of (S)- and (R)-isomers. In some embodiments, the (R)-isomers in the mixture of identical chemical entities (i.e., mixture of stereoisomers) is present at an (R)-enantiomeric excess of about 10% to about 99.5%, about 20% to about 99.5%, about 30% to about 99.5%, about 40% to about 99.5%, about 50% to about 99.5%, about 55% to about 99.5%, about 60% to about 99.5%, about 65% to about 99.5%, about 70% to about 99.5%, about 75% to about 99.5%, about 80% to about 99.5%, about 85% to about 99.5%, about 90% to about 99.5%, about 95% to about 99.5%, about 96% to about 99.5%, about 97% to about 99.5%, about 98% to about 99.5%, or about 99% to about 99.5%, or more than about 99.5%.

Enantiomers can be isolated from racemic mixtures by any method known to those skilled in the art, including chiral high pressure liquid chromatography (HPLC), the formation and crystallization of chiral salts, or prepared by asymmetric syntheses. See, for example, Enantiomers, Racemates and Resolutions (Jacques, Ed., Wiley Interscience, New York, 1981); Wilen et al., Tetrahedron 33:2725 (1977); Stereochemistry of Carbon Compounds (E. L. Eliel, Ed., McGraw-Hill, NY, 1962); and Tables of Resolving Agents and Optical Resolutions p. 268 (E. L. Eliel, Ed., Univ. of Notre Dame Press, Notre Dame, IN 1972).

When a range of values is listed, it is intended to encompass each value and subrange within the range. For example, “C₁₋₆” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆.

As used herein, the phrase “administration” of a compound, “administering” a compound, or other variants thereof means providing the compound or a prodrug of the compound to the individual in need of treatment.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art.

The term “tautomers” or “tautomeric” refers to two or more interconvertible compounds resulting from tautomerization. The exact ratio of the tautomers depends on several factors, including for example temperature, solvent, and pH. Tautomerizations are known to those skilled in the art. Exemplary tautomerizations include keto-to-enol, amide-to-imide, lactam-to-lactim, enamine-to-imine, and enamine-to-(a different enamine) tautomerizations.

The term “subject” (alternatively referred to herein as “patient”) as used herein, refers to an animal, such as a mammal, such as a human, who has been the object of treatment, observation or experiment.

As used herein, the terms “treat,” “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated. As used herein, the terms “treat,” “treating,” “treatment,” and the like may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition. The term “treat” and synonyms contemplate administering a therapeutically effective amount of a compound described herein to a subject in need of such treatment.

The term “effective amount” refers to that amount of a compound or combination of compounds as described herein that is sufficient to achieve the intended effect, including, but not limited to, treatment of diseases. A therapeutically effective amount may vary depending upon the intended application (in vitro or in vivo), or the subject and disease condition being treated (e.g., the weight, age and gender of the subject), the severity of the disease condition, the manner of administration, etc. which can readily be determined by one of ordinary skill in the art. The term also applies to a dose that will induce a particular response in target cells and/or tissues. The specific dose will vary depending on the particular compounds chosen, the dosing regimen to be followed, whether the compound is administered in combination with other compounds, timing of administration, the tissue to which it is administered, and the physical delivery system in which the compound is carried.

As used herein, the singular form “a”, “an”, and “the”, includes plural references unless it is expressly stated or is unambiguously clear from the context that such is not intended.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the terms “comprising” and “including” can be used interchangeably. The terms “comprising” and “including” are to be interpreted as specifying the presence of the stated features or components as referred to but does not preclude the presence or addition of one or more features, or components, or groups thereof. Additionally, the terms “comprising” and “including” are intended to include examples encompassed by the term “consisting of”. Consequently, the term “consisting of” can be used in place of the terms “comprising” and “including” to provide for more specific embodiments.

As used herein, and unless otherwise specified, the term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions as provided herein is contemplated. Supplementary active ingredients can also be incorporated into the pharmaceutical compositions.

As used herein, and unless otherwise indicated, the term “about” or “approximately” refers to an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

Headings and subheadings are used for convenience and/or formal compliance only, do not limit the subject technology, and are not referred to in connection with the interpretation of the description of the subject technology. Features described under one heading or one subheading of the subject application may be combined, in various embodiments, with features described under other headings or subheadings. Further it is not necessarily the case that all features under a single heading or a single subheading are used together in embodiments.

### Compounds

Provided herein are isotopologues of certain aminoheteroaryl compounds. In some embodiments, the compounds are capable of selectively inhibiting one or more Cyclin-dependent kinases (CDKs). In some embodiments, the compounds are capable of selectively inhibiting Cyclin-dependent kinase 2 (CDK2) over other CDKs.

Isotopic enrichment (e.g., deuteration) of pharmaceuticals to improve pharmacokinetics (“PK”), pharmacodynamics (“PD”), and toxicity profiles, has been demonstrated previously with some classes of drugs. (See, e.g., Lijinsky et. al., Food Cosmet. Toxicol., 20: 393 (1982); Lijinsky et. al., J. Nat. Cancer Inst., 69: 1127 (1982); Mangold et. al., Mutation Res. 308: 33 (1994); Gordon et. al., Drug Metab. Dispos., 15: 589 (1987); Zello et. al., Metabolism, 43: 487 (1994); Gately et. al., J. Nucl. Med., 27: 388 (1986); Wade D, Chem. Biol. Interact. 117: 191 (1999)).

Without being limited by a particular theory, isotopic enrichment of a drug can be used, for example, to (1) reduce or eliminate unwanted metabolites, (2) increase the half-life of the parent drug, (3) decrease the number of doses needed to achieve a desired effect, (4) decrease the amount of a dose necessary to achieve a desired effect, (5) increase the formation of active metabolites, if any are formed, and/or (6) decrease the production of deleterious metabolites in specific tissues and/or create a more effective drug and/or a safer drug for combination therapy, whether the combination therapy is intentional or not.

Replacement of an atom for one of its isotopes may often result in a change in the reaction rate of a chemical reaction. This phenomenon is known as the Kinetic Isotope Effect (“KIE”). For example, if a C—H bond is broken during a rate-determining step in a chemical reaction (i.e., the step with the highest transition state energy), substitution of a deuterium for that hydrogen will cause a decrease in the reaction rate and the process will slow down. This phenomenon is known as the Deuterium Kinetic Isotope Effect (“DKIE”). (See, e.g, Foster et al., Adv. Drug Res., vol. 14, pp. 1-36 (1985); Kushner et al., Can. J. Physiol. Pharmacol., vol. 77, pp. 79-88 (1999)).

The magnitude of the DKIE can be expressed as the ratio between the rates of a given reaction in which a C—H bond is broken, and the same reaction where deuterium is substituted for hydrogen. The DKIE can range from about 1 (no isotope effect) to very large numbers, such as 50 or more, meaning that the reaction can be fifty, or more, times slower when deuterium is substituted for hydrogen. Without being limited by a particular theory, high DKIE values may be due in part to a phenomenon known as tunneling, which is a consequence of the uncertainty principle. Tunneling is ascribed to the small mass of a hydrogen atom, and occurs because transition states involving a proton can sometimes form in the absence of the required activation energy. Because deuterium has more mass than hydrogen, it statistically has a much lower probability of undergoing this phenomenon.

Tritium (“T”) is a radioactive isotope of hydrogen, used in research, fusion reactors, neutron generators and radiopharmaceuticals. Tritium is a hydrogen atom that has 2 neutrons in the nucleus and has an atomic weight close to 3. It occurs naturally in the environment in very low concentrations, most commonly found as T₂O. Tritium decays slowly (half-life=12.3 years) and emits a low energy beta particle that cannot penetrate the outer layer of human skin. Internal exposure is the main hazard associated with this isotope, yet it must be ingested in large amounts to pose a significant health risk. As compared with deuterium, a lesser amount of tritium must be consumed before it reaches a hazardous level. Substitution of tritium (“T”) for hydrogen results in yet a stronger bond than deuterium and gives numerically larger isotope effects. Similarly, substitution of isotopes for other elements, including, but not limited to, ¹³C or ¹⁴C for carbon, ³³S, ³⁴S, or ³⁶S for sulfur, ¹⁵N for nitrogen, and ¹⁷O or ¹⁸O for oxygen, may lead to a similar kinetic isotope effect.

The animal body expresses a variety of enzymes for the purpose of eliminating foreign substances, such as therapeutic agents, from its circulation system. Examples of such enzymes include the cytochrome P450 enzymes (“CYPs”), esterases, proteases, reductases, dehydrogenases, and monoamine oxidases, to react with and convert these foreign substances to more polar intermediates or metabolites for renal excretion. Some of the most common metabolic reactions of pharmaceutical compounds involve the oxidation of a carbon-hydrogen (C—H) bond to either a carbon-oxygen (C—O) or carbon-carbon (C—C) pi-bond. The resultant metabolites may be stable or unstable under physiological conditions, and can have substantially different pharmacokinetic, pharmacodynamic, and acute and long-term toxicity profiles relative to the parent compounds. For many drugs, such oxidations are rapid. These drugs therefore often require the administration of multiple or high daily doses.

Therefore, isotopic enrichment at certain positions of a compound provided herein may produce a detectable KIE that affects the pharmacokinetic, pharmacologic, and/or toxicological profiles of a compound provided herein in comparison with a similar compound having a natural isotopic composition. In certain embodiments, the deuterium enrichment is performed on the site of C—H bond cleavage during metabolism.

In some embodiments, provided herein are isotopologues of 4-((4-(((3S,4R)-3-hydroxytetrahydro-2H-pyran-4-yl)oxy)-5-(trifluoromethyl)pyrimidin-2-yl)amino)-N-methylbenzenesulfonamide, in which one or more hydrogen positions of the molecule is/are isotopically enriched with deuterium. In some embodiments, provided here are isotopologues of 4-((4-(((3S,4R)-3-hydroxytetrahydro-2H-pyran-4-yl)oxy)-5-(trifluoromethyl)pyrimidin-2-yl)amino)-N-(methyl-d₃)benzenesulfonamide, in which one or more hydrogen positions of the molecule (in addition to methyl-d₃) is/are isotopically enriched with deuterium.

In some embodiments, provided herein is a compound of Formula (I):

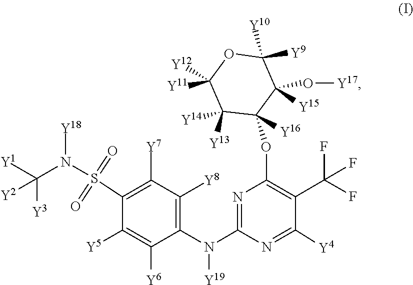

or a stereoisomer, a mixture of stereoisomers, tautomer, or a pharmaceutically acceptable salt thereof, wherein at least one of Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, Y¹⁶, Y¹⁷, Y¹⁸, and Y¹⁹ is a hydrogen that is isotopically enriched with deuterium (“D”), and the others of Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, Y¹⁶, Y¹⁷, Y¹⁸, and Y¹⁹ are non-enriched hydrogen atoms (“H”), provided that the compound is not

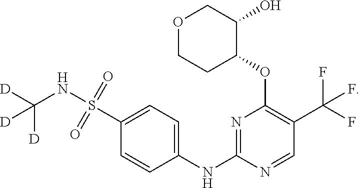 [SMILES: [2H]C([2H])([2H])NS(=O)(=O)c1ccc(Nc2ncc(C(F)(F)F)c(O[C@@H]3CCOC[C@@H]3O)n2)cc1]

In some embodiments, one or more Y atoms on the methyl group (i.e., Y¹, Y², and Y³) of Compound of Formula (I) are deuterium-enriched. In some embodiments, the Y atom on the pyrimidine ring (i.e., Y⁴) of Compound of Formula (I) is deuterium-enriched. In some embodiments, one or more Y atoms on the benzene ring (i.e., Y⁵, Y⁶, Y⁷, and Y⁸) of Compound of Formula (I) are deuterium-enriched. In some embodiments, one or more Y atoms on the tetrahydro-2H-pyran ring (i.e., Y⁹, Y¹⁰, Y¹¹, Y¹², Y¹³, Y¹⁴, Y¹⁵, and Y¹⁶) of Compound of Formula (I) are deuterium-enriched. In some embodiments, one or more exchangeable Y atoms (i.e., Y¹⁷, Y¹⁸, and Y¹⁹) of Compound of Formula (I) are deuterium-enriched. In some embodiments, one or more Y atoms on both the methyl group and benzene ring of Formula (I) are deuterium-enriched, i.e., any combination of deuteration shown above for the methyl group and the benzene ring is encompassed. For example, particular compounds provided herein include the following listed compounds in Tables 1-6, or an enantiomer or a mixture of enantiomers thereof, in which the label “D” indicates a deuterium-enriched atomic position, i.e., a sample comprising the given compound has a deuterium enrichment at the indicated position(s) above the natural abundance of deuterium.

In some embodiments, the compound is a compound of Formula (II):

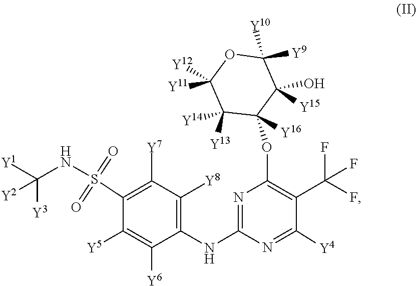

or a stereoisomer, a mixture of stereoisomers, tautomer, or a pharmaceutically acceptable salt thereof, wherein at least one of Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, and Y¹⁶ is a hydrogen that is isotopically enriched with deuterium, and the others of Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, and Y¹⁶ are non-enriched hydrogen atoms, provided that the compound is not

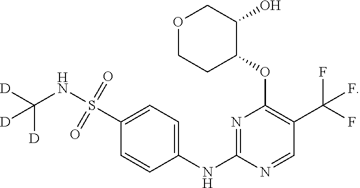 [SMILES: [2H]C([2H])([2H])NS(=O)(=O)c1ccc(Nc2ncc(C(F)(F)F)c(O[C@@H]3CCOC[C@@H]3O)n2)cc1]

In some embodiments, Y¹, Y², and Y³ are all H. In some embodiments, Y¹ is D, and Y² and Y³ are H. In some embodiments, Y¹ and Y² are D, and Y³ is H. In some embodiments, Y¹, Y², and Y³ are all D.

In some embodiments, the compound is a compound of Formula (III):

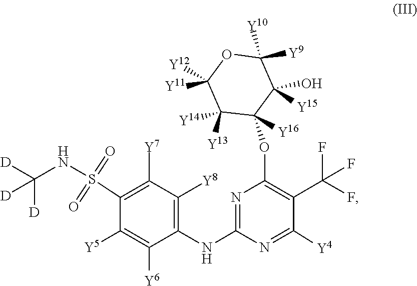

or a stereoisomer, a mixture of stereoisomers, tautomer, or a pharmaceutically acceptable salt thereof, wherein at least one of Y⁴, Y⁵, Y⁶, Y⁷, Y⁸, Y⁹, Y¹⁰, Y¹¹, Y¹², Y¹³, Y¹⁴, Y¹⁵, and Y¹⁶ is a hydrogen that is isotopically enriched with deuterium, and the others of Y⁴, Y⁵, Y⁶, Y⁷, Y⁸, Y⁹, Y¹⁰, Y¹¹, Y¹², Y¹³, Y¹⁴, Y¹⁵, and Y¹⁶ are non-enriched hydrogen atoms.

In some embodiments, the compound is a compound of Formula (I), Formula (II), or Formula (III) and one of Y⁴, Y⁵, Y⁶, Y⁷, Y⁸, Y⁹, Y¹⁰, Y¹¹, Y¹², Y¹³, Y¹⁴, Y¹⁵, and Y¹⁶ is a hydrogen that is isotopically enriched with deuterium, and the others of Y⁴, Y⁵, Y⁶, Y⁷, Y⁸, Y⁹, Y¹⁰, Y¹¹, Y¹², Y¹³, Y¹⁴, Y¹⁵, and Y¹⁶ are non-enriched hydrogen atoms.

In some embodiments, the compound is a compound of Formula (I), Formula (II), or Formula (III) and two of Y⁴, Y⁵, Y⁶, Y⁷, Y⁸, Y⁹, Y¹⁰, Y¹¹, Y¹², Y¹³, Y¹⁴, Y¹⁵, and Y¹⁶ are hydrogen atoms that are isotopically enriched with deuterium, and the others of Y⁴, Y⁵, Y⁶, Y⁷, Y⁸, Y⁹, Y¹⁰, Y¹¹, Y¹², Y¹³, Y¹⁴, Y¹⁵, and Y¹⁶ are non-enriched hydrogen atoms.

In some embodiments, the compound is a compound of Formula (I), Formula (II), or Formula (III) and three of Y⁴, Y⁵, Y⁶, Y⁷, Y⁸, Y⁹, Y¹⁰, Y¹¹, Y¹², Y¹³, Y¹⁴, Y¹⁵, and Y¹⁶ are hydrogen atoms that are isotopically enriched with deuterium, and the others of Y⁴, Y⁵, Y⁶, Y⁷, Y⁸, Y⁹, Y¹⁰, Y¹¹, Y¹², Y¹³, Y¹⁴, Y¹¹⁵ and Y¹⁶ are non-enriched hydrogen atoms.

In some embodiments, the compound is a compound of Formula (I), Formula (II), or Formula (III) and four of Y⁴, Y⁵, Y⁶, Y⁷, Y⁸, Y⁹, Y¹⁰, Y¹¹, Y¹², Y¹³, Y¹⁴, Y¹⁵, and Y¹⁶ are hydrogen atoms that are isotopically enriched with deuterium, and the others of Y⁴, Y⁵, Y⁶, Y⁷, Y⁸, Y⁹, Y¹⁰, Y¹¹, Y¹², Y¹³, Y¹⁴, Y¹⁵ and Y¹⁶ are non-enriched hydrogen atoms.

In some embodiments, the compound is a compound of Formula (I), Formula (II), or Formula (III) and five of Y⁴, Y⁵, Y⁶, Y⁷, Y⁸, Y⁹, Y¹⁰, Y¹¹, Y¹², Y¹³, Y¹⁴, Y¹⁵, and Y¹⁶ are hydrogen atoms that are isotopically enriched with deuterium, and the others of Y⁴, Y⁵, Y⁶, Y⁷, Y⁸, Y⁹, Y¹⁰, Y¹¹, Y¹², Y¹³, Y¹⁴, Y¹⁵ and Y¹⁶ are non-enriched hydrogen atoms.

In some embodiments, the compound is a compound of Formula (I), Formula (II), or Formula (III) and six of Y⁴, Y⁵, Y⁶, Y⁷, Y⁸, Y⁹, Y¹⁰, Y¹¹, Y¹², Y¹³, Y¹⁴, Y¹⁵, and Y¹⁶ are hydrogen atoms that are isotopically enriched with deuterium, and the others of Y⁴, Y⁵, Y⁶, Y⁷, Y⁸, Y⁹, Y¹⁰, Y¹¹, Y¹², Y¹³, Y¹⁴, Y¹⁵, and Y¹⁶ are non-enriched hydrogen atoms.

In some embodiments, the compound is a compound of Formula (I), Formula (II), or Formula (III) and seven of Y⁴, Y⁵, Y⁶, Y⁷, Y⁸, Y⁹, Y¹⁰, Y¹¹, Y¹², Y¹³, Y¹⁴, Y¹⁵, and Y¹⁶ are hydrogen atoms that are isotopically enriched with deuterium, and the others of Y⁴, Y⁵, Y⁶, Y⁷, Y⁸, Y⁹, Y¹⁰, Y¹¹, Y¹², Y¹³, Y¹⁴, Y¹⁵, and Y¹⁶ are non-enriched hydrogen atoms.

In some embodiments, the compound is a compound of Formula (I), Formula (II), or Formula (III) and eight of Y⁴, Y⁵, Y⁶, Y⁷, Y⁸, Y⁹, Y¹⁰, Y¹¹, Y¹², Y¹³, Y¹⁴, Y¹⁵, and Y¹⁶ are hydrogen atoms that are isotopically enriched with deuterium, and the others of Y⁴, Y⁵, Y⁶, Y⁷, Y⁸, Y⁹, Y¹⁰, Y¹¹, Y¹², Y¹³, Y¹⁴, Y¹⁵ and Y¹⁶ are non-enriched hydrogen atoms.

In some embodiments, the compound is a compound of Formula (I), Formula (II), or Formula (III) and nine of Y⁴, Y⁵, Y⁶, Y⁷, Y⁸, Y⁹, Y¹⁰, Y¹¹, Y¹², Y¹³, Y¹⁴, Y¹⁵, and Y¹⁶ are hydrogen atoms that are isotopically enriched with deuterium, and the others of Y⁴, Y⁵, Y⁶, Y⁷, Y⁸, Y⁹, Y¹⁰, Y¹¹, Y¹², Y¹³, Y¹⁴, Y¹⁵ and Y¹⁶ are non-enriched hydrogen atoms.

In some embodiments, the compound is a compound of Formula (I), Formula (II), or Formula (III) and ten of Y⁴, Y⁵, Y⁶, Y⁷, Y⁸, Y⁹, Y¹⁰, Y¹¹, Y¹², Y¹³, Y¹⁴, Y¹⁵, and Y¹⁶ are hydrogen atoms that are isotopically enriched with deuterium, and the others of Y⁴, Y⁵, Y⁶, Y⁷, Y⁸, Y⁹, Y¹⁰, Y¹¹, Y¹², Y¹³, Y¹⁴, Y¹⁵ and Y¹⁶ are non-enriched hydrogen atoms.

In some embodiments, the compound is a compound of Formula (I), Formula (II), or Formula (III) and eleven of Y⁴, Y⁵, Y⁶, Y⁷, Y⁸, Y⁹, Y¹⁰, Y¹¹, Y¹², Y¹³, Y¹⁴, Y¹⁵, and Y¹⁶ are hydrogen atoms that are isotopically enriched with deuterium, and the others of Y⁴, Y⁵, Y⁶, Y⁷, Y⁸, Y⁹, Y¹⁰, Y¹¹, Y¹², Y¹³, Y¹⁴, Y¹⁵ and Y¹⁶ are non-enriched hydrogen atoms.

In some embodiments, the compound is a compound of Formula (I), Formula (II), or Formula (III) and twelve of Y⁴, Y⁵, Y⁶, Y⁷, Y⁸, Y⁹, Y¹⁰, Y¹¹, Y¹², Y¹³, Y¹⁴, Y¹⁵, and Y¹⁶ are hydrogen atoms that are isotopically enriched with deuterium, and the other of Y⁴, Y⁵, Y⁶, Y⁷, Y⁸, Y⁹, Y¹⁰, Y¹¹, Y¹², Y¹³, Y¹⁴, Y¹⁵, and Y¹⁶ is non-enriched hydrogen atoms.

In some embodiments, the compound is a compound of Formula (I), Formula (II), or Formula (III) and all of Y⁴, Y⁵, Y⁶, Y⁷, Y⁸, Y⁹, Y¹⁰, Y¹¹, Y¹², Y¹³, Y¹⁴, Y¹⁵, and Y¹⁶ are hydrogen atoms that are isotopically enriched with deuterium.

In some embodiments, the compound is a compound of Formula (I), Formula (II), or Formula (III) and Y⁴ is D.

In some embodiments, the compound is a compound of Formula (I) or Formula (II) and Y¹, Y², Y³, and Y⁴ are all D.

In some embodiments, the compound is a compound of Formula (III-A), (III-B), (III-C), (III-D), (III-E), (III-F), (III-G), or (III-H).

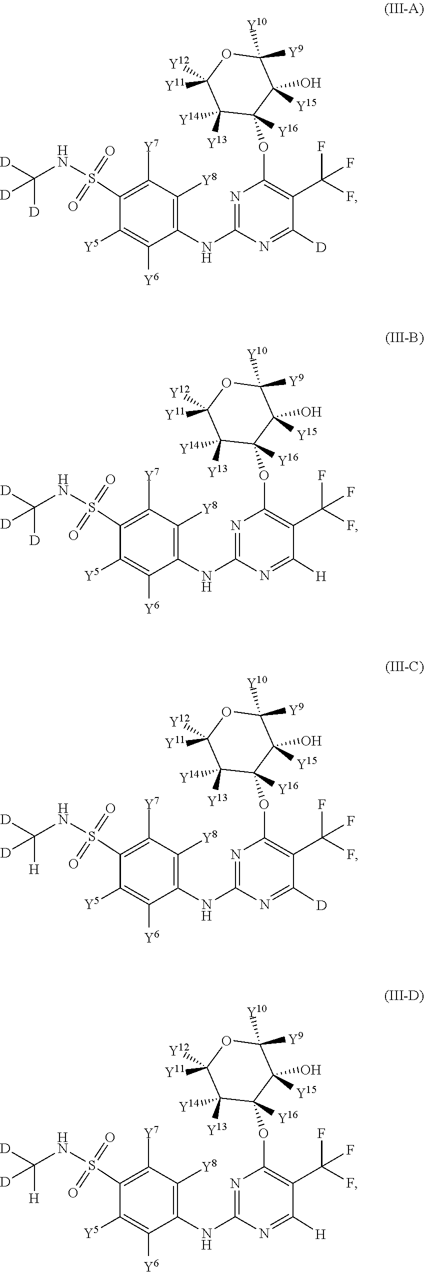

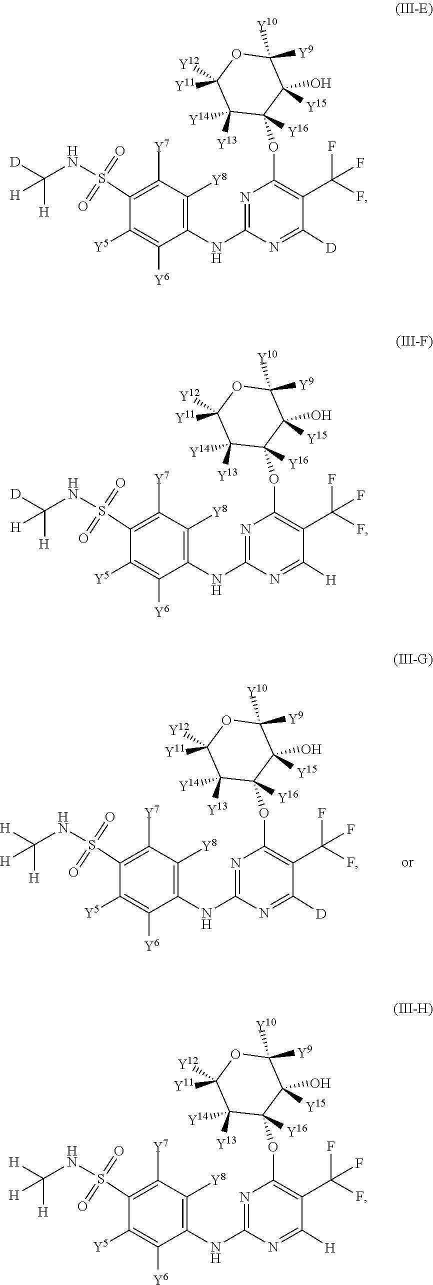

or a stereoisomer, a mixture of stereoisomers, tautomer, or a pharmaceutically acceptable salt thereof.

In some embodiments, the compound is a compound of Formula (III-A), (III-B), (III-C), (III-D), (III-E), (III-F), (III-G), or (III-H), wherein at least one of Y⁵, Y⁶, Y⁷, Y⁸, Y⁹, Y¹⁰, Y¹¹, Y¹², Y¹³, Y¹⁴, Y¹⁵, and Y¹⁶ is a hydrogen that is isotopically enriched with deuterium, and the others of Y⁵, Y⁶, Y⁷, Y⁸, Y⁹, Y¹⁰, Y¹¹, Y¹², Y¹³, Y¹⁴, Y¹⁵, and Y¹⁶ are non-enriched hydrogen atoms.

In some embodiments, the compound is a compound of Formula (III-A), (III-B), (III-C), (III-D), (III-E), (III-F), (III-G), or (III-H), wherein one of Y⁵, Y⁶, Y⁷, Y⁸, Y⁹, Y¹⁰, Y¹¹, Y¹², Y¹³, Y¹⁴, Y¹⁵, and Y¹⁶ is a hydrogen that is isotopically enriched with deuterium, and the others of Y⁵, Y⁶, Y⁷, Y⁸, Y⁹, Y¹⁰, Y¹¹, Y¹², Y¹³, Y¹⁴, Y¹⁵, and Y¹⁶ are non-enriched hydrogen atoms.

In some embodiments, the compound is a compound of Formula (III-A), (III-B), (III-C), (III-D), (III-E), (III-F), (III-G), or (III-H), wherein two of Y⁵, Y⁶, Y⁷, Y⁸, Y⁹, Y¹⁰, Y¹¹, Y¹², Y¹³, Y¹⁴, Y¹⁵, and Y¹⁶ are hydrogen atoms that are isotopically enriched with deuterium, and the others of Y⁵, Y⁶, Y⁷, Y⁸, Y⁹, Y¹⁰, Y¹¹, Y¹², Y¹³, Y¹⁴, Y¹⁵, and Y¹⁶ are non-enriched hydrogen atoms.

In some embodiments, the compound is a compound of Formula (III-A), (III-B), (III-C), (III-D), (III-E), (III-F), (III-G), or (III-H), wherein three of Y⁵, Y⁶, Y⁷, Y⁸, Y⁹, Y¹⁰, Y¹¹, Y¹², Y¹³, Y¹⁴, Y¹⁵, and Y¹⁶ are hydrogen atoms that are isotopically enriched with deuterium, and the others of Y⁵, Y⁶, Y⁷, Y⁸, Y⁹, Y¹⁰, Y¹¹, Y¹², Y¹³, Y¹⁴, Y¹⁵, and Y¹⁶ are non-enriched hydrogen atoms.

In some embodiments, the compound is a compound of Formula (III-A), (III-B), (III-C), (III-D), (III-E), (III-F), (III-G), or (III-H), wherein four of Y⁵, Y⁶, Y⁷, Y⁸, Y⁹, Y¹⁰, Y¹¹, Y¹², Y¹³, Y¹⁴, Y¹⁵, and Y¹⁶ are hydrogen atoms that are isotopically enriched with deuterium, and the others of Y⁵, Y⁶, Y⁷, Y⁸, Y⁹, Y¹⁰, Y¹¹, Y¹², Y¹³, Y¹⁴, Y¹⁵, and Y¹⁶ are non-enriched hydrogen atoms.

In some embodiments, the compound is a compound of Formula (III-A), (III-B), (III-C), (III-D), (III-E), (III-F), (III-G), or (III-H), wherein five of Y⁵, Y⁶, Y⁷, Y⁸, Y⁹, Y¹⁰, Y¹¹, Y¹², Y¹³, Y¹⁴, Y¹⁵, and Y¹⁶ are hydrogen atoms that are isotopically enriched with deuterium, and the others of Y⁵, Y⁶, Y⁷, Y⁸, Y⁹, Y¹⁰, Y¹¹, Y¹², Y¹³, Y¹⁴, Y¹⁵, and Y¹⁶ are non-enriched hydrogen atoms.

In some embodiments, the compound is a compound of Formula (III-A), (III-B), (III-C), (III-D), (III-E), (III-F), (III-G), or (III-H), wherein six of Y⁵, Y⁶, Y⁷, Y⁸, Y⁹, Y¹⁰, Y¹¹, Y¹², Y¹³, Y¹⁴, Y¹⁵, and Y¹⁶ are hydrogen atoms that are isotopically enriched with deuterium, and the others of Y⁵, Y⁶, Y⁷, Y⁸, Y⁹, Y¹⁰, Y¹¹, Y¹², Y¹³, Y¹⁴, Y¹⁵, and Y¹⁶ are non-enriched hydrogen atoms.

In some embodiments, the compound is a compound of Formula (III-A), (III-B), (III-C), (III-D), (III-E), (III-F), (III-G), or (III-H), wherein seven of Y⁵, Y⁶, Y⁷, Y⁸, Y⁹, Y¹⁰, Y¹¹, Y¹², Y¹³, Y¹⁴, Y¹⁵, and Y¹⁶ are hydrogen atoms that are isotopically enriched with deuterium, and the others of Y⁵, Y⁶, Y⁷, Y⁸, Y⁹, Y¹⁰, Y¹¹, Y¹², Y¹³, Y¹⁴, Y¹⁵, and Y¹⁶ are non-enriched hydrogen atoms.

In some embodiments, the compound is a compound of Formula (III-A), (III-B), (III-C), (III-D), (III-E), (III-F), (III-G), or (III-H), wherein eight of Y⁵, Y⁶, Y⁷, Y⁸, Y⁹, Y¹⁰, Y¹¹, Y¹², Y¹³, Y¹⁴, Y¹⁵, and Y¹⁶ are hydrogen atoms that are isotopically enriched with deuterium, and the others of Y⁵, Y⁶, Y⁷, Y⁸, Y⁹, Y¹⁰, Y¹¹, Y¹², Y¹³, Y¹⁴, Y¹⁵, and Y¹⁶ are non-enriched hydrogen atoms.

In some embodiments, the compound is a compound of Formula (III-A), (III-B), (III-C), (III-D), (III-E), (III-F), (III-G), or (III-H), wherein nine of Y⁵, Y⁶, Y⁷, Y⁸, Y⁹, Y¹⁰, Y¹¹, Y¹², Y¹³, Y¹⁴, Y¹⁵, and Y¹⁶ are hydrogen atoms that are isotopically enriched with deuterium, and the others of Y⁵, Y⁶, Y⁷, Y⁸, Y⁹, Y¹⁰, Y¹¹, Y¹², Y¹³, Y¹⁴, Y¹⁵, and Y¹⁶ are non-enriched hydrogen atoms.

In some embodiments, the compound is a compound of Formula (III-A), (III-B), (III-C), (III-D), (III-E), (III-F), (III-G), or (III-H), wherein ten of Y⁵, Y⁶, Y⁷, Y⁸, Y⁹, Y¹⁰, Y¹¹, Y¹², Y¹³, Y¹⁴, Y¹⁵, and Y¹⁶ are hydrogen atoms that are isotopically enriched with deuterium, and the others of Y⁵, Y⁶, Y⁷, Y⁸, Y⁹, Y¹⁰, Y¹¹, Y¹², Y¹³, Y¹⁴, Y¹⁵, and Y¹⁶ are non-enriched hydrogen atoms.

In some embodiments, the compound is a compound of Formula (III-A), (III-B), (III-C), (III-D), (III-E), (III-F), (III-G), or (III-H), wherein eleven of Y⁵, Y⁶, Y⁷, Y⁸, Y⁹, Y¹⁰, Y¹¹, Y¹², Y¹³, Y¹⁴, Y¹⁵, and Y¹⁶ are hydrogen atoms that are isotopically enriched with deuterium, and the others of Y⁵, Y⁶, Y⁷, Y⁸, Y⁹, Y¹⁰, Y¹¹, Y¹², Y¹³, Y¹⁴, Y¹⁵, and Y¹⁶ are non-enriched hydrogen atoms.

In some embodiments, the compound is a compound of Formula (III-A), (III-B), (III-C), (III-D), (III-E), (III-F), (III-G), or (III-H), wherein all of Y⁵, Y⁶, Y⁷, Y⁸, Y⁹, Y¹⁰, Y¹¹, Y¹², Y¹³, Y¹⁴, Y¹⁵, and Y¹⁶ are hydrogen atoms that are isotopically enriched with deuterium.

In some embodiments, the compound is a compound of Formula (IV):

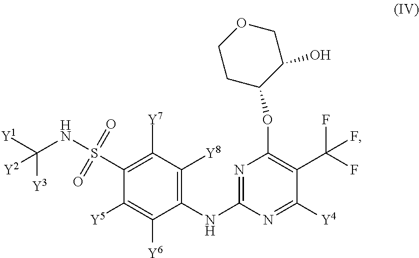

or a stereoisomer, a mixture of stereoisomers, tautomer, or a pharmaceutically acceptable salt thereof, wherein at least one of Y¹, Y², Y³, Y⁴, Y⁵, Y⁶, Y⁷, and Y⁸ is a hydrogen that is isotopically enriched with deuterium, and the others of Y¹, Y², Y³, Y⁴, Y⁵, Y⁶, Y⁷, and Y⁸ are non-enriched hydrogen atoms, provided that the compound is not

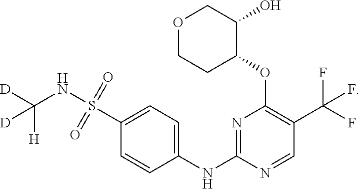 [SMILES: [2H]C([2H])NS(=O)(=O)c1ccc(Nc2ncc(C(F)(F)F)c(O[C@@H]3CCOC[C@@H]3O)n2)cc1]

In some embodiments, the compound is a compound of Formula (IV), wherein one of Y¹, Y², Y³, Y⁴, Y⁵, Y⁶, Y⁷, and Y⁸ is a hydrogen that is isotopically enriched with deuterium, and the others of Y¹, Y², Y³, Y⁴, Y⁵, Y⁶, Y⁷, and Y⁸ are non-enriched hydrogen atoms.

In some embodiments, the compound is a compound of Formula (IV), wherein two of Y¹, Y², Y³, Y⁴, Y⁵, Y⁶, Y⁷, and Y⁸ are hydrogen atoms that are that is isotopically enriched with deuterium, and the others of Y¹, Y², Y³, Y⁴, Y⁵, Y⁶, Y⁷, and Y⁸ are non-enriched hydrogen atoms.

In some embodiments, the compound is a compound of Formula (IV), wherein three of Y¹, Y², Y³, Y⁴, Y⁵, Y⁶, Y⁷, and Y⁸ are hydrogen atoms that are isotopically enriched with deuterium, and the others of Y¹, Y², Y³, Y⁴, Y⁵, Y⁶, Y⁷, and Y⁸ are non-enriched hydrogen atoms.

In some embodiments, the compound is a compound of Formula (IV), wherein four of Y¹, Y², Y³, Y⁴, Y⁵, Y⁶, Y⁷, and Y⁸ are hydrogen atoms that are isotopically enriched with deuterium, and the others of Y¹, Y², Y³, Y⁴, Y⁵, Y⁶, Y⁷, and Y⁸ are non-enriched hydrogen atoms.

In some embodiments, the compound is a compound of Formula (IV), wherein five of Y¹, Y², Y³, Y⁴, Y⁵, Y⁶, Y⁷, and Y⁸ are hydrogen atoms that are isotopically enriched with deuterium, and the others of Y¹, Y², Y³, Y⁴, Y⁵, Y⁶, Y⁷, and Y⁸ are non-enriched hydrogen atoms.

In some embodiments, the compound is a compound of Formula (IV), wherein six of Y¹, Y², Y³, Y⁴, Y⁵, Y⁶, Y⁷, and Y⁸ are hydrogen atoms that are isotopically enriched with deuterium, and the others of Y¹, Y², Y³, Y⁴, Y⁵, Y⁶, Y⁷, and Y⁸ are non-enriched hydrogen atoms.

In some embodiments, the compound is a compound of Formula (IV), wherein seven of Y, Y², Y³, Y⁴, Y⁵, Y⁶, Y⁷, and Y⁸ are hydrogen atoms that are isotopically enriched with deuterium, and the others of Y¹, Y², Y³, Y⁴, Y⁵, Y⁶, Y⁷, and Y⁸ are non-enriched hydrogen atoms.

In some embodiments, the compound is a compound of Formula (IV), wherein all of Y¹, Y², Y³, Y⁴, Y⁵, Y⁶, Y⁷, and Y⁸ are hydrogen atoms that are isotopically enriched with deuterium.

In some embodiments, the compound is a compound of Formula (V):

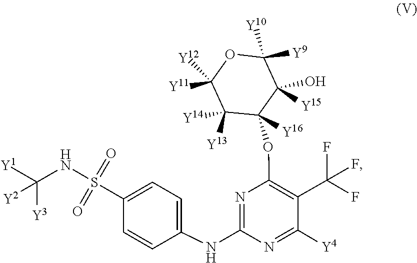

or a stereoisomer, a mixture of stereoisomers, tautomer, or a pharmaceutically acceptable salt thereof, wherein at least one of Y¹, Y², Y³, Y⁴, Y⁹, Y¹⁰, Y¹¹, Y¹², Y¹³, Y¹⁴, Y¹⁵, and Y¹⁶ is a hydrogen that is isotopically enriched with deuterium, and the others of Y¹, Y², Y³, Y⁴⁴, Y⁹, Y¹⁰, Y¹², Y¹³, Y¹⁴, Y¹⁵, and Y¹⁶ are non-enriched hydrogen atoms, provided that the compound is not

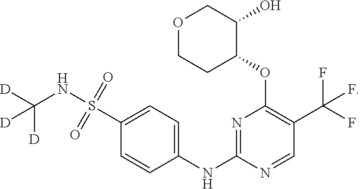 [SMILES: [2H]C([2H])([2H])NS(=O)(=O)c1ccc(Nc2ncc(C(F)(F)F)c(O[C@@H]3CCOC[C@@H]3O)n2)cc1]

In some embodiments, the compound is a compound of Formula (V), wherein one of Y¹, Y², Y³, Y⁴, Y⁹, Y¹⁰, Y¹¹, Y¹², Y¹³, Y¹⁴, Y¹⁵, and Y¹⁶ is a hydrogen that is isotopically enriched with deuterium, and the others of Y¹, Y², Y³, Y⁴, Y⁹, Y¹⁰, Y¹¹, Y¹², Y¹³, Y¹⁴, Y¹⁵, and Y¹⁶ are non-enriched hydrogen atoms.

In some embodiments, the compound is a compound of Formula (V), wherein two of Y¹, Y², Y³, Y⁴, Y⁹, Y¹⁰, Y¹¹, Y¹², Y¹³, Y¹⁴, Y¹⁵, and Y¹⁶ are hydrogen atoms that are isotopically enriched with deuterium, and the others of Y¹, Y², Y³, Y⁴, Y⁹, Y¹⁰, Y¹¹, Y¹², Y¹³, Y¹⁴, Y¹⁵, and Y¹⁶ are non-enriched hydrogen atoms.

In some embodiments, the compound is a compound of Formula (V), wherein three of Y¹, Y², Y³, Y⁴, Y⁹, Y¹⁰, Y¹¹, Y¹², Y¹³, Y¹⁴, Y¹⁵, and Y¹⁶ are hydrogen atoms that are isotopically enriched with deuterium, and the others of Y¹, Y², Y³, Y⁴, Y⁹, Y¹⁰, Y¹¹, Y¹², Y¹³, Y¹⁴, Y¹⁵, and Y¹⁶ are non-enriched hydrogen atoms.

In some embodiments, the compound is a compound of Formula (V), wherein four of Y¹, Y², Y³, Y⁴, Y⁹, Y¹⁰, Y¹¹, Y¹², Y¹³, Y¹⁴, Y¹⁵, and Y¹⁶ are hydrogen atoms that are isotopically enriched with deuterium, and the others of Y¹, Y², Y³, Y⁴, Y⁹, Y¹⁰, Y¹¹, Y¹², Y¹³, Y¹⁴, Y¹⁵, and Y¹⁶ are non-enriched hydrogen atoms.

In some embodiments, the compound is a compound of Formula (V), wherein five of Y¹, Y², Y³, Y⁴, Y⁹, Y¹⁰, Y¹¹, Y¹², Y¹³, Y¹⁴, Y¹⁵, and Y¹⁶ are hydrogen atoms that are isotopically enriched with deuterium, and the others of Y¹, Y², Y³, Y⁴, Y⁹, Y¹⁰, Y¹¹, Y¹², Y¹³, Y¹⁴, Y¹⁵, and Y¹⁶ are non-enriched hydrogen atoms.

In some embodiments, the compound is a compound of Formula (V), wherein six of Y¹, Y², Y³, Y⁴, Y⁹, Y¹⁰, Y¹¹, Y¹², Y¹³, Y¹⁴, Y¹⁵, and Y¹⁶ are hydrogen atoms that are isotopically enriched with deuterium, and the others of Y¹, Y², Y³, Y⁴, Y⁹, Y¹⁰, Y¹¹, Y¹², Y¹³, Y¹⁴, Y¹⁵, and Y¹⁶ are non-enriched hydrogen atoms.

In some embodiments, the compound is a compound of Formula (V), wherein seven of Y¹, Y², Y³, Y⁴, Y⁹, Y¹⁰, Y¹¹, Y¹², Y¹³, Y¹⁴, Y¹⁵, and Y¹⁶ are hydrogen atoms that are isotopically enriched with deuterium, and the others of Y¹, Y², Y³, Y⁴, Y⁹, Y¹⁰, Y¹¹, Y¹², Y¹³, Y¹⁴, Y¹, and Y¹⁶ are non-enriched hydrogen atoms.

In some embodiments, the compound is a compound of Formula (V), wherein eight of Y¹, Y², Y³, Y⁴, Y⁹, Y¹⁰, Y¹¹, Y¹², Y¹³, Y¹⁴, Y¹⁵, and Y¹⁶ are hydrogen atoms that are isotopically enriched with deuterium, and the others of Y¹, Y², Y³, Y⁴, Y⁹, Y¹⁰, Y¹¹, Y¹², Y¹³, Y¹⁴, Y¹⁵, and Y¹⁶ are non-enriched hydrogen atoms.

In some embodiments, the compound is a compound of Formula (V), wherein nine of Y¹, Y², Y³, Y⁴, Y⁹, Y¹⁰, Y¹¹, Y¹², Y¹³, Y¹⁴, Y¹⁵, and Y¹⁶ are hydrogen atoms that are isotopically enriched with deuterium, and the others of Y¹, Y², Y³, Y⁴, Y⁹, Y¹⁰, Y¹¹, Y¹², Y¹³, Y¹⁴, Y¹⁵, and Y¹⁶ are non-enriched hydrogen atoms.

In some embodiments, the compound is a compound of Formula (V), wherein ten of Y¹, Y², Y³, Y⁴, Y⁹, Y¹⁰, Y¹¹, Y¹², Y¹³, Y¹⁴, Y¹⁵, and Y¹⁶ are hydrogen atoms that are isotopically enriched with deuterium, and the others of Y¹, Y², Y³, Y⁴, Y⁹, Y¹⁰, Y¹¹, Y¹², Y¹³, Y¹⁴, Y¹⁵, and Y¹⁶ are non-enriched hydrogen atoms.

In some embodiments, the compound is a compound of Formula (V), wherein eleven of Y¹, Y², Y³, Y⁴, Y⁹, Y¹⁰, Y¹¹, Y¹², Y¹³, Y¹⁴, Y¹⁵, and Y¹⁶ are hydrogen atoms that are isotopically enriched with deuterium, and the others of Y¹, Y², Y³, Y⁴, Y⁹, Y¹⁰, Y¹¹, Y¹², Y¹³, Y¹⁴, Y¹⁵, and Y¹⁶ are non-enriched hydrogen atoms.

In some embodiments, the compound is a compound of Formula (V), wherein all of Y¹, Y², Y³, Y⁴, Y⁹, Y¹⁰, Y¹¹, Y¹², Y¹³, Y¹⁴, Y¹⁵, and Y¹⁶ are hydrogen atoms that are isotopically enriched with deuterium.

In some embodiments, the compound is a compound of Formula (VI):

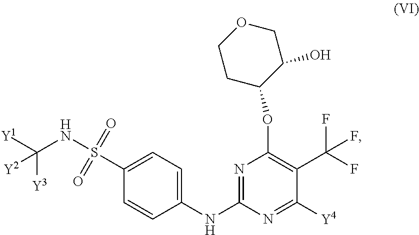

or a stereoisomer, a mixture of stereoisomers, tautomer, or a pharmaceutically acceptable salt thereof, wherein at least one of Y¹, Y², Y³, and Y⁴ is a hydrogen that is isotopically enriched with deuterium, and the others of Y¹, Y², Y³, and Y⁴ are non-enriched hydrogen atoms, provided that the compound is not

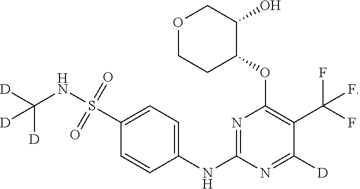 [SMILES: [2H]c1nc(Nc2ccc(S(=O)(=O)NC([2H])([2H])[2H])cc2)nc(O[C@@H]2CCOC[C@@H]2O)c1C(F)(F)F]

In some embodiments, the compound is a compound of Formula (VI), wherein one of Y¹, Y², Y³, and Y⁴ is a hydrogen atom that is isotopically enriched with deuterium, and the others of Y¹, Y², Y³, and Y⁴ are non-enriched hydrogen atoms.

In some embodiments, the compound is a compound of Formula (VI), wherein two of Y¹, Y², Y³, and Y⁴ are hydrogen atoms that are isotopically enriched with deuterium, and the others of Y¹, Y², Y³, and Y⁴ are non-enriched hydrogen atoms.

In some embodiments, the compound is a compound of Formula (VI), wherein three of Y¹, Y², Y³, and Y⁴ are hydrogen atoms that are isotopically enriched with deuterium, and the others of Y¹, Y², Y³, and Y⁴ are non-enriched hydrogen atoms.

In some embodiments, the compound is a compound of Formula (VI), wherein all of Y¹, Y², Y³, and Y⁴ are hydrogen atoms that are isotopically enriched with deuterium.

In some embodiments, the compound is selected from any one of the compounds in Tables 1 to 6, or a stereoisomer, a mixture of stereoisomers, tautomer, or a pharmaceutically acceptable salt thereof.

In some embodiments, the compound is:

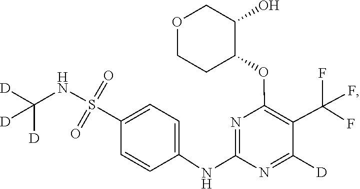 [SMILES: [2H]c1nc(Nc2ccc(S(=O)(=O)NC([2H])([2H])[2H])cc2)nc(O[C@@H]2CCOC[C@@H]2O)c1C(F)(F)F]

or a pharmaceutically acceptable salt thereof.

In some embodiments, the phenyl moiety of the compound

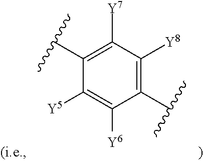

is

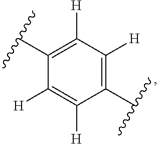 [SMILES: *c1ccc(*)cc1]

wherein the attachment to the left is to the —SO₂— group. In some embodiments, the phenyl moiety is

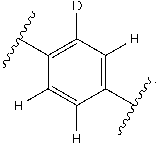 [SMILES: *c1ccc(*)c([2H])c1]

In some embodiments, the phenyl moiety is

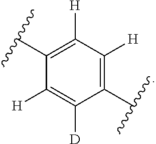 [SMILES: *c1ccc(*)c([2H])c1]

In some embodiments, the phenyl moiety is

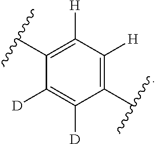 [SMILES: *c1ccc(*)c([2H])c1[2H]]

In some embodiments, the phenyl moiety is

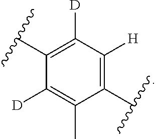 [SMILES: *c1cc([2H])c(*)c([2H])c1C]

In some embodiments, the phenyl moiety is

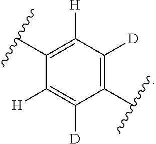 [SMILES: *c1cc([2H])c(*)c([2H])c1]

In some embodiments, the phenyl moiety is

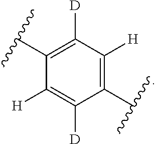 [SMILES: *c1cc([2H])c(*)cc1[2H]]

In some embodiments, the phenyl moiety is

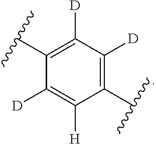 [SMILES: *c1cc([2H])c(*)c([2H])c1[2H]]

In some embodiments, the phenyl moiety is

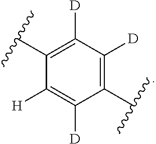 [SMILES: *c1cc([2H])c(*)c([2H])c1[2H]]

In some embodiments, the phenyl moiety is

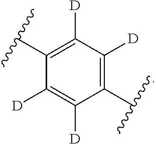 [SMILES: *c1c([2H])c([2H])c(*)c([2H])c1[2H]]

In some embodiments, the pyrimidine moiety of the compound

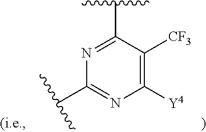

is

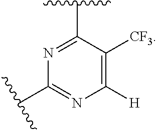 [SMILES: *c1ncc(C(F)(F)F)c(*)n1]

In some embodiments, the pyrimidine moiety is

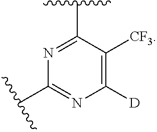 [SMILES: *c1nc(*)c(C(F)(F)F)c([2H])n1]

In some embodiments, the tetrahydropyran moiety of the compound

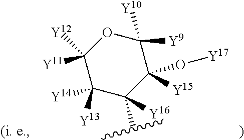

is

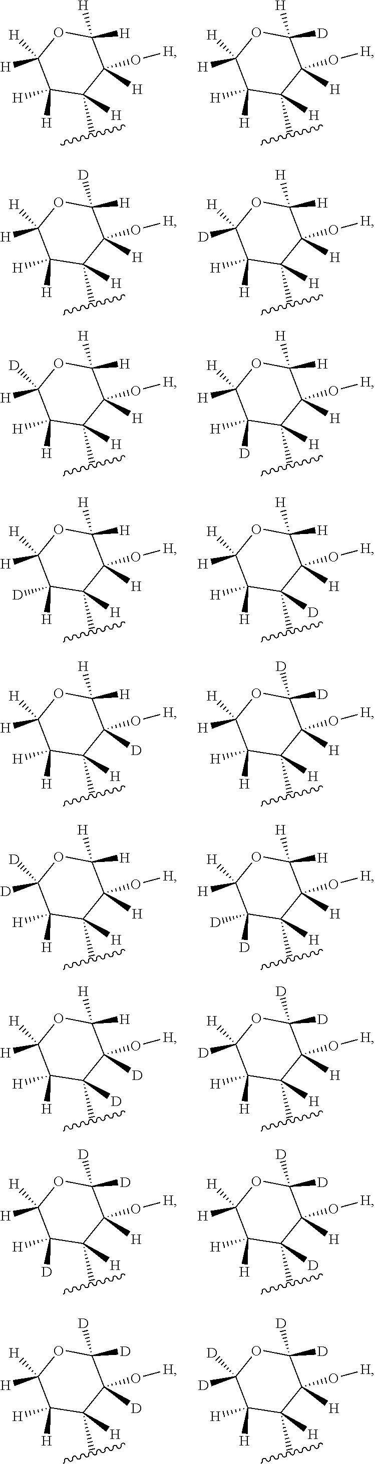

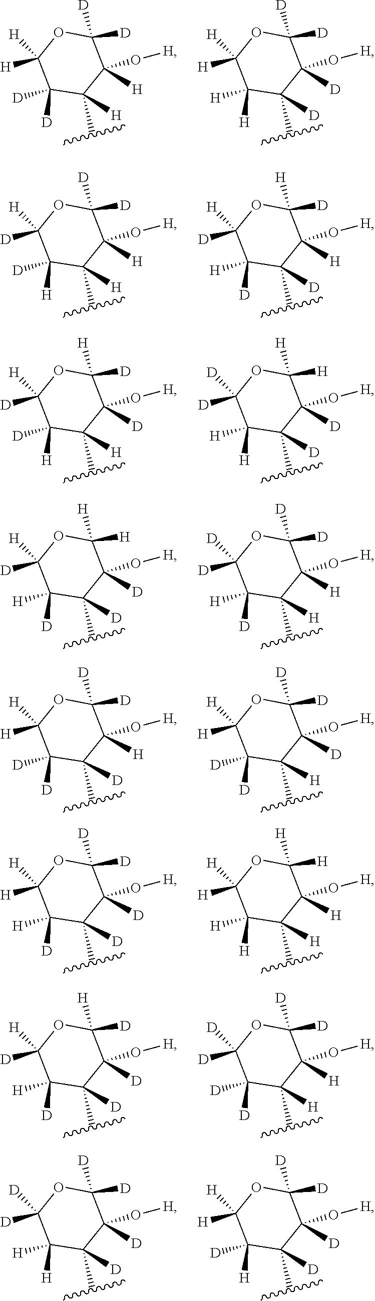

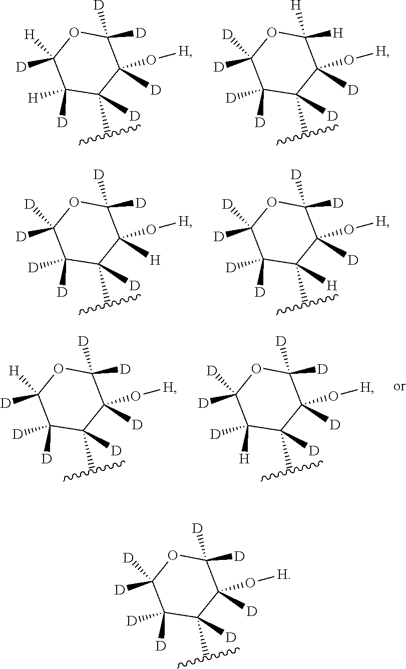

In some embodiments, the tetrahydropyran moiety is

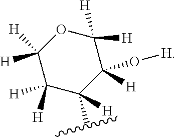

In some embodiments, the tetrahydropyran moiety is

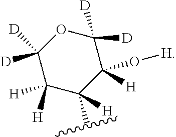

In some embodiments, the tetrahydropyran moiety is

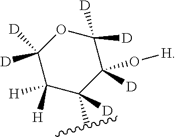

In some embodiments, the tetrahydropyran moiety is

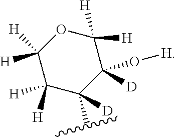

In some embodiments, the tetrahydropyran moiety is

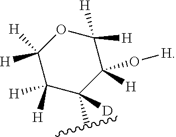

In some embodiments, the tetrahydropyran moiety is

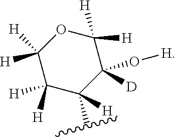

All combinations of the phenyl moiety, the pyrimidine moiety, and the tetrahydropyran moiety are specifically provided herein. All additional combinations with —CH₃, —CH₂D, —CHD₂, or -CD₃ (i.e., the methyl group on the sulfonamide) are also specifically provided herein.

In some embodiments, the compound is a compound of Formula (I) selected from any one of the compounds in Tables 1-4, or a stereoisomer, a mixture of stereoisomers, tautomer, or a pharmaceutically acceptable salt thereof.

TABLE 1
Deuterium enriched compounds of Formula (I):

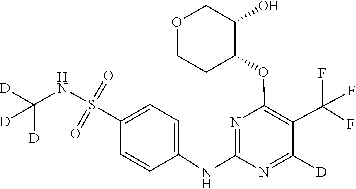 [SMILES: [2H]c1nc(Nc2ccc(S(=O)(=O)NC([2H])([2H])[2H])cc2)nc(O[C@@H]2CCOC[C@@H]2O)c1C(F)(F)F]

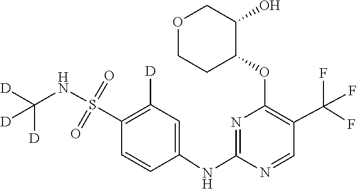 [SMILES: [2H]c1cc(Nc2ncc(C(F)(F)F)c(O[C@@H]3CCOC[C@@H]3O)n2)ccc1S(=O)(=O)NC([2H])([2H])[2H]]

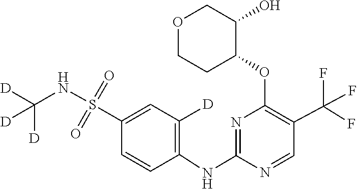 [SMILES: [2H]c1cc(S(=O)(=O)NC([2H])([2H])[2H])ccc1Nc1ncc(C(F)(F)F)c(O[C@@H]2CCOC[C@@H]2O)n1]

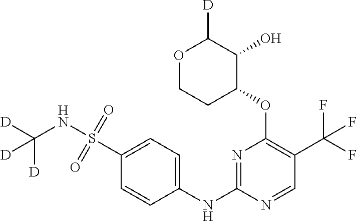 [SMILES: [2H]C1OCC[C@@H](Oc2nc(Nc3ccc(S(=O)(=O)NC([2H])([2H])[2H])cc3)ncc2C(F)(F)F)[C@H]1O]

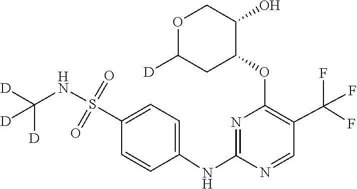 [SMILES: [2H]C1C[C@@H](Oc2nc(Nc3ccc(S(=O)(=O)NC([2H])([2H])[2H])cc3)ncc2C(F)(F)F)[C@@H](O)CO1]

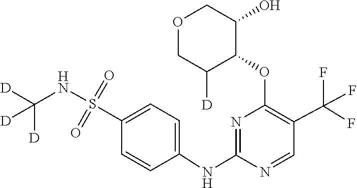 [SMILES: [2H]C1COC[C@H](O)[C@@H]1Oc1nc(Nc2ccc(S(=O)(=O)NC([2H])([2H])[2H])cc2)ncc1C(F)(F)F]

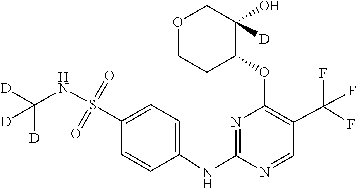 [SMILES: [2H]C([2H])([2H])NS(=O)(=O)c1ccc(Nc2ncc(C(F)(F)F)c(O[C@@H]3CCOC[C@]3([2H])O)n2)cc1]

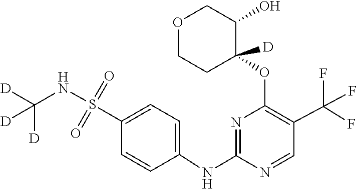 [SMILES: [2H]C([2H])([2H])NS(=O)(=O)c1ccc(Nc2ncc(C(F)(F)F)c(O[C@]3([2H])CCOC[C@@H]3O)n2)cc1]

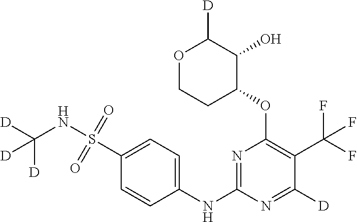 [SMILES: [2H]c1nc(Nc2ccc(S(=O)(=O)NC([2H])([2H])[2H])cc2)nc(O[C@@H]2CCOC([2H])[C@@H]2O)c1C(F)(F)F]

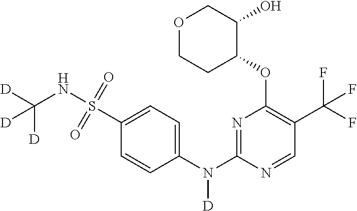

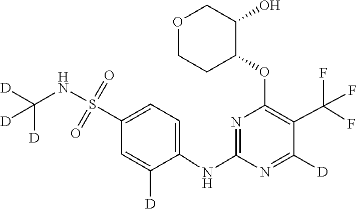 [SMILES: [2H]c1cc(S(=O)(=O)NC([2H])([2H])[2H])ccc1Nc1nc([2H])c(C(F)(F)F)c(O[C@@H]2CCOC[C@@H]2O)n1]

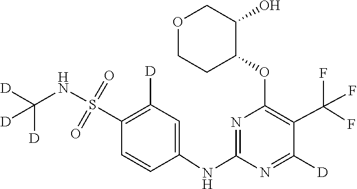 [SMILES: [2H]c1cc(Nc2nc([2H])c(C(F)(F)F)c(O[C@@H]3CCOC[C@@H]3O)n2)ccc1S(=O)(=O)NC([2H])([2H])[2H]]

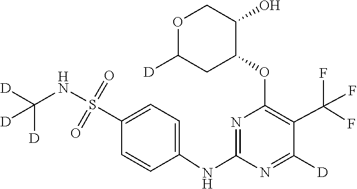 [SMILES: [2H]c1nc(Nc2ccc(S(=O)(=O)NC([2H])([2H])[2H])cc2)nc(O[C@@H]2CC([2H])OC[C@@H]2O)c1C(F)(F)F]

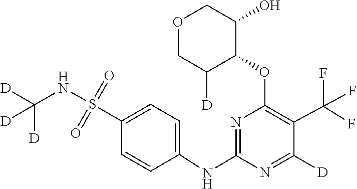 [SMILES: [2H]c1nc(Nc2ccc(S(=O)(=O)NC([2H])([2H])[2H])cc2)nc(O[C@@H]2C([2H])COC[C@@H]2O)c1C(F)(F)F]

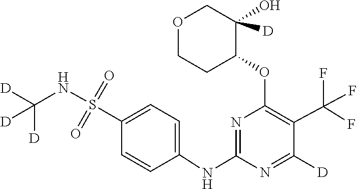 [SMILES: [2H]c1nc(Nc2ccc(S(=O)(=O)NC([2H])([2H])[2H])cc2)nc(O[C@@H]2CCOC[C@]2([2H])O)c1C(F)(F)F]

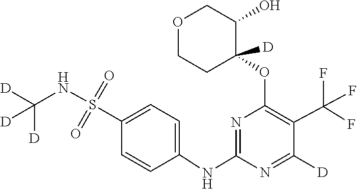 [SMILES: [2H]c1nc(Nc2ccc(S(=O)(=O)NC([2H])([2H])[2H])cc2)nc(O[C@]2([2H])CCOC[C@@H]2O)c1C(F)(F)F]

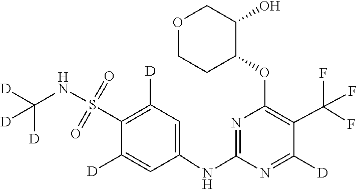 [SMILES: [2H]c1cc(Nc2nc([2H])c(C(F)(F)F)c(O[C@@H]3CCOC[C@@H]3O)n2)cc([2H])c1S(=O)(=O)NC([2H])([2H])[2H]]

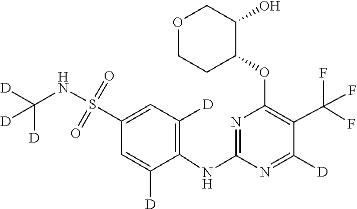 [SMILES: [2H]c1cc(S(=O)(=O)NC([2H])([2H])[2H])cc([2H])c1Nc1nc([2H])c(C(F)(F)F)c(O[C@@H]2CCOC[C@@H]2O)n1]

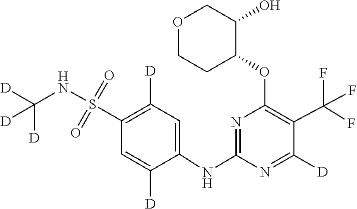 [SMILES: [2H]c1cc(S(=O)(=O)NC([2H])([2H])[2H])c([2H])cc1Nc1nc([2H])c(C(F)(F)F)c(O[C@@H]2CCOC[C@@H]2O)n1]

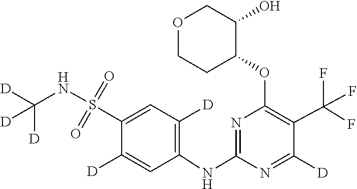 [SMILES: [2H]c1cc(S(=O)(=O)NC([2H])([2H])[2H])c([2H])cc1Nc1nc([2H])c(C(F)(F)F)c(O[C@@H]2CCOC[C@@H]2O)n1]

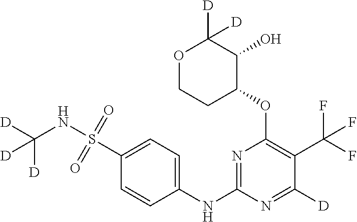 [SMILES: [2H]c1nc(Nc2ccc(S(=O)(=O)NC([2H])([2H])[2H])cc2)nc(O[C@@H]2CCOC([2H])([2H])[C@@H]2O)c1C(F)(F)F]

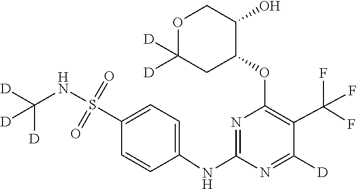 [SMILES: [2H]c1nc(Nc2ccc(S(=O)(=O)NC([2H])([2H])[2H])cc2)nc(O[C@@H]2CC([2H])([2H])OC[C@@H]2O)c1C(F)(F)F]

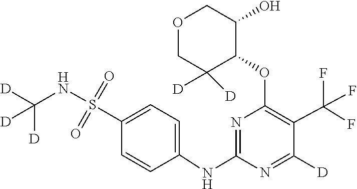 [SMILES: [2H]c1nc(Nc2ccc(S(=O)(=O)NC([2H])([2H])[2H])cc2)nc(O[C@H]2[C@@H](O)COCC2([2H])[2H])c1C(F)(F)F]

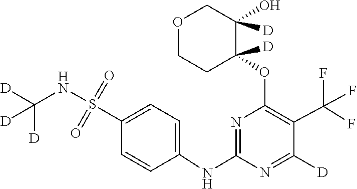 [SMILES: [2H]c1nc(Nc2ccc(S(=O)(=O)NC([2H])([2H])[2H])cc2)nc(O[C@]2([2H])CCOC[C@]2([2H])O)c1C(F)(F)F]

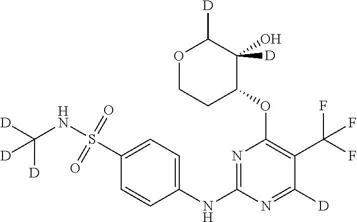 [SMILES: [2H]c1nc(Nc2ccc(S(=O)(=O)NC([2H])([2H])[2H])cc2)nc(O[C@@H]2CCOC([2H])[C@]2([2H])O)c1C(F)(F)F]

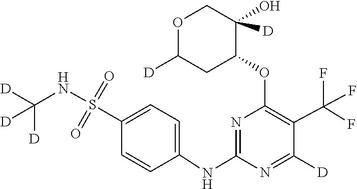 [SMILES: [2H]c1nc(Nc2ccc(S(=O)(=O)NC([2H])([2H])[2H])cc2)nc(O[C@@H]2CC([2H])OC[C@]2([2H])O)c1C(F)(F)F]

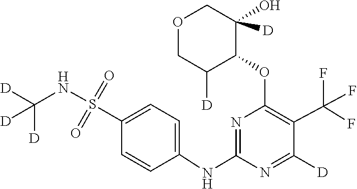 [SMILES: [2H]c1nc(Nc2ccc(S(=O)(=O)NC([2H])([2H])[2H])cc2)nc(O[C@@H]2C([2H])COC[C@]2([2H])O)c1C(F)(F)F]

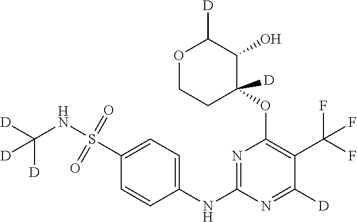 [SMILES: [2H]c1nc(Nc2ccc(S(=O)(=O)NC([2H])([2H])[2H])cc2)nc(O[C@]2([2H])CCOC([2H])[C@@H]2O)c1C(F)(F)F]

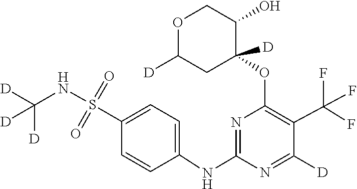 [SMILES: [2H]c1nc(Nc2ccc(S(=O)(=O)NC([2H])([2H])[2H])cc2)nc(O[C@]2([2H])CC([2H])OC[C@@H]2O)c1C(F)(F)F]

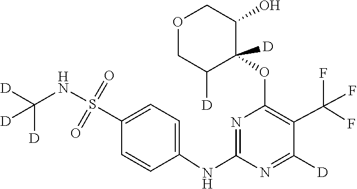 [SMILES: [2H]c1nc(Nc2ccc(S(=O)(=O)NC([2H])([2H])[2H])cc2)nc(O[C@]2([2H])C([2H])COC[C@@H]2O)c1C(F)(F)F]

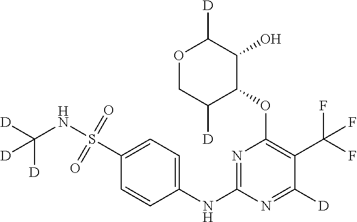 [SMILES: [2H]c1nc(Nc2ccc(S(=O)(=O)NC([2H])([2H])[2H])cc2)nc(O[C@@H]2C([2H])COC([2H])[C@@H]2O)c1C(F)(F)F]

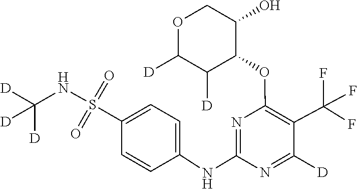 [SMILES: [2H]c1nc(Nc2ccc(S(=O)(=O)NC([2H])([2H])[2H])cc2)nc(O[C@@H]2C([2H])C([2H])OC[C@@H]2O)c1C(F)(F)F]

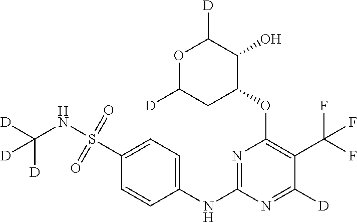 [SMILES: [2H]c1nc(Nc2ccc(S(=O)(=O)NC([2H])([2H])[2H])cc2)nc(O[C@@H]2CC([2H])OC([2H])[C@@H]2O)c1C(F)(F)F]

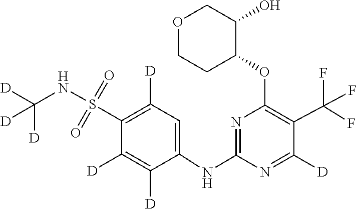 [SMILES: [2H]c1cc(Nc2nc([2H])c(C(F)(F)F)c(O[C@@H]3CCOC[C@@H]3O)n2)c([2H])c([2H])c1S(=O)(=O)NC([2H])([2H])[2H]]

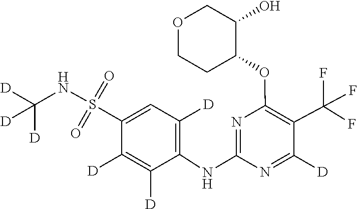 [SMILES: [2H]c1cc(S(=O)(=O)NC([2H])([2H])[2H])c([2H])c([2H])c1Nc1nc([2H])c(C(F)(F)F)c(O[C@@H]2CCOC[C@@H]2O)n1]

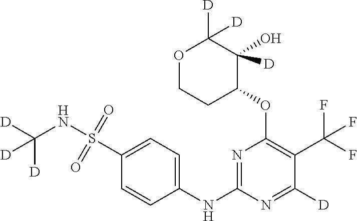 [SMILES: [2H]c1nc(Nc2ccc(S(=O)(=O)NC([2H])([2H])[2H])cc2)nc(O[C@@H]2CCOC([2H])([2H])[C@]2([2H])O)c1C(F)(F)F]

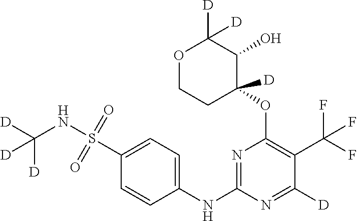 [SMILES: [2H]c1nc(Nc2ccc(S(=O)(=O)NC([2H])([2H])[2H])cc2)nc(O[C@]2([2H])CCOC([2H])([2H])[C@@H]2O)c1C(F)(F)F]

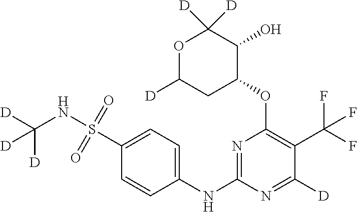 [SMILES: [2H]c1nc(Nc2ccc(S(=O)(=O)NC([2H])([2H])[2H])cc2)nc(O[C@@H]2CC([2H])OC([2H])([2H])[C@@H]2O)c1C(F)(F)F]

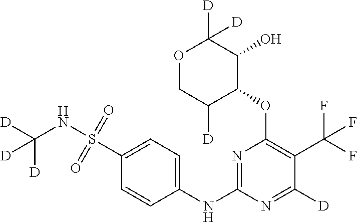 [SMILES: [2H]c1nc(Nc2ccc(S(=O)(=O)NC([2H])([2H])[2H])cc2)nc(O[C@@H]2C([2H])COC([2H])([2H])[C@@H]2O)c1C(F)(F)F]

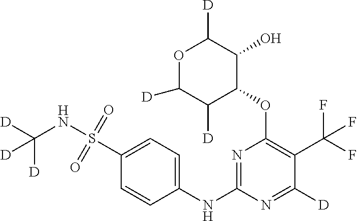 [SMILES: [2H]c1nc(Nc2ccc(S(=O)(=O)NC([2H])([2H])[2H])cc2)nc(O[C@@H]2C([2H])C([2H])OC([2H])[C@@H]2O)c1C(F)(F)F]

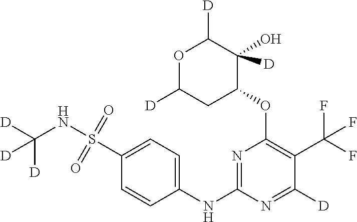 [SMILES: [2H]c1nc(Nc2ccc(S(=O)(=O)NC([2H])([2H])[2H])cc2)nc(O[C@@H]2CC([2H])OC([2H])[C@]2([2H])O)c1C(F)(F)F]

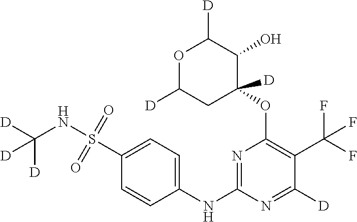 [SMILES: [2H]c1nc(Nc2ccc(S(=O)(=O)NC([2H])([2H])[2H])cc2)nc(O[C@]2([2H])CC([2H])OC([2H])[C@@H]2O)c1C(F)(F)F]

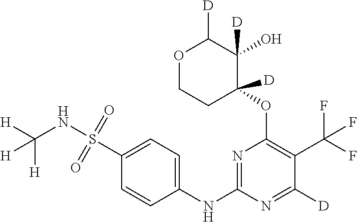 [SMILES: [2H]c1nc(Nc2ccc(S(=O)(=O)NC)cc2)nc(O[C@]2([2H])CCOC([2H])[C@]2([2H])O)c1C(F)(F)F]

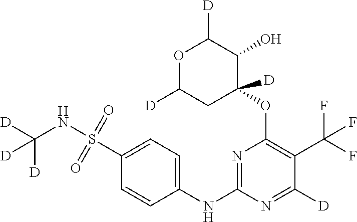 [SMILES: [2H]c1nc(Nc2ccc(S(=O)(=O)NC([2H])([2H])[2H])cc2)nc(O[C@]2([2H])CC([2H])OC([2H])[C@@H]2O)c1C(F)(F)F]

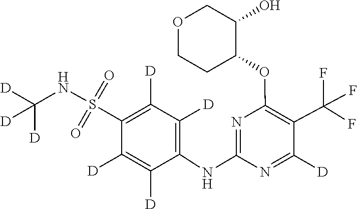 [SMILES: [2H]c1nc(Nc2c([2H])c([2H])c(S(=O)(=O)NC([2H])([2H])[2H])c([2H])c2[2H])nc(O[C@@H]2CCOC[C@@H]2O)c1C(F)(F)F]

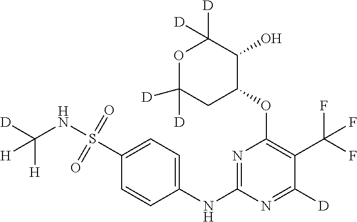 [SMILES: [2H]CNS(=O)(=O)c1ccc(Nc2nc([2H])c(C(F)(F)F)c(O[C@@H]3CC([2H])([2H])OC([2H])([2H])[C@@H]3O)n2)cc1]

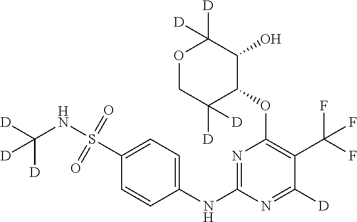 [SMILES: [2H]c1nc(Nc2ccc(S(=O)(=O)NC([2H])([2H])[2H])cc2)nc(O[C@H]2[C@@H](O)C([2H])([2H])OCC2([2H])[2H])c1C(F)(F)F]

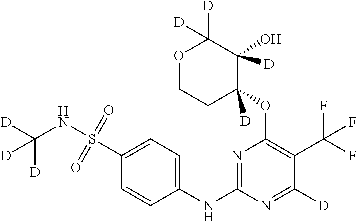 [SMILES: [2H]c1nc(Nc2ccc(S(=O)(=O)NC([2H])([2H])[2H])cc2)nc(O[C@]2([2H])CCOC([2H])([2H])[C@]2([2H])O)c1C(F)(F)F]

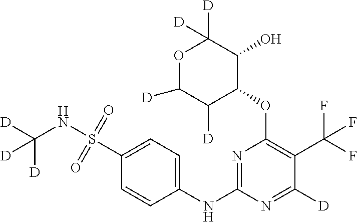 [SMILES: [2H]c1nc(Nc2ccc(S(=O)(=O)NC([2H])([2H])[2H])cc2)nc(O[C@@H]2C([2H])C([2H])OC([2H])([2H])[C@@H]2O)c1C(F)(F)F]

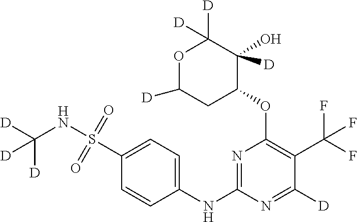 [SMILES: [2H]c1nc(Nc2ccc(S(=O)(=O)NC([2H])([2H])[2H])cc2)nc(O[C@@H]2CC([2H])OC([2H])([2H])[C@]2([2H])O)c1C(F)(F)F]

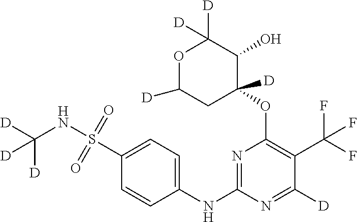 [SMILES: [2H]c1nc(Nc2ccc(S(=O)(=O)NC([2H])([2H])[2H])cc2)nc(O[C@]2([2H])CC([2H])OC([2H])([2H])[C@@H]2O)c1C(F)(F)F]

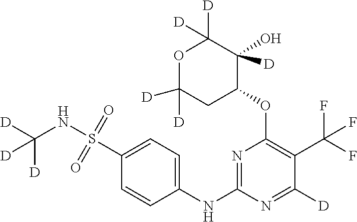 [SMILES: [2H]c1nc(Nc2ccc(S(=O)(=O)NC([2H])([2H])[2H])cc2)nc(O[C@@H]2CC([2H])([2H])OC([2H])([2H])[C@]2([2H])O)c1C(F)(F)F]

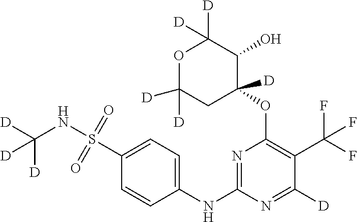 [SMILES: [2H]c1nc(Nc2ccc(S(=O)(=O)NC([2H])([2H])[2H])cc2)nc(O[C@]2([2H])CC([2H])([2H])OC([2H])([2H])[C@@H]2O)c1C(F)(F)F]

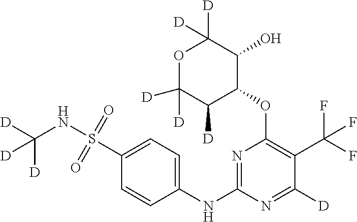 [SMILES: [2H]c1nc(Nc2ccc(S(=O)(=O)NC([2H])([2H])[2H])cc2)nc(O[C@@H]2[C@@H]([2H])C([2H])([2H])OC([2H])([2H])[C@@H]2O)c1C(F)(F)F]

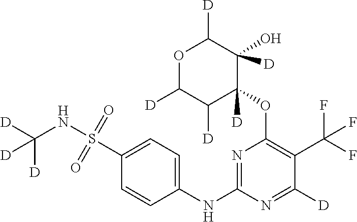 [SMILES: [2H]c1nc(Nc2ccc(S(=O)(=O)NC([2H])([2H])[2H])cc2)nc(O[C@]2([2H])C([2H])C([2H])OC([2H])[C@]2([2H])O)c1C(F)(F)F]

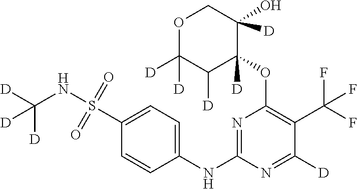 [SMILES: [2H]c1nc(Nc2ccc(S(=O)(=O)NC([2H])([2H])[2H])cc2)nc(O[C@]2([2H])C([2H])C([2H])([2H])OC[C@]2([2H])O)c1C(F)(F)F]

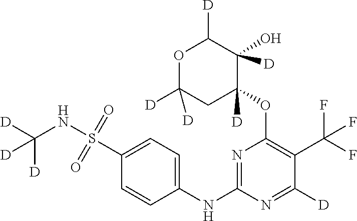 [SMILES: [2H]c1nc(Nc2ccc(S(=O)(=O)NC([2H])([2H])[2H])cc2)nc(O[C@]2([2H])CC([2H])([2H])OC([2H])[C@]2([2H])O)c1C(F)(F)F]

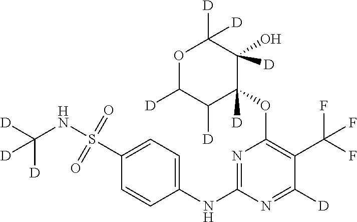 [SMILES: [2H]c1nc(Nc2ccc(S(=O)(=O)NC([2H])([2H])[2H])cc2)nc(O[C@]2([2H])C([2H])C([2H])OC([2H])([2H])[C@]2([2H])O)c1C(F)(F)F]

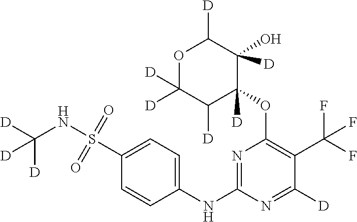 [SMILES: [2H]c1nc(Nc2ccc(S(=O)(=O)NC([2H])([2H])[2H])cc2)nc(O[C@]2([2H])C([2H])C([2H])([2H])OC([2H])[C@]2([2H])O)c1C(F)(F)F]

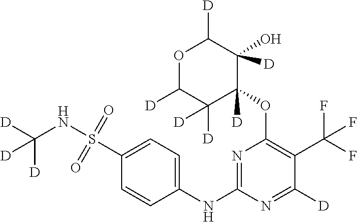 [SMILES: [2H]c1nc(Nc2ccc(S(=O)(=O)NC([2H])([2H])[2H])cc2)nc(O[C@]2([2H])C([2H])([2H])C([2H])OC([2H])[C@]2([2H])O)c1C(F)(F)F]

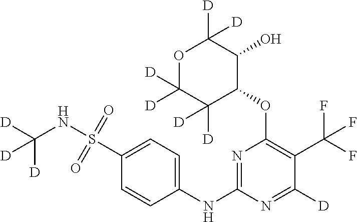 [SMILES: [2H]c1nc(Nc2ccc(S(=O)(=O)NC([2H])([2H])[2H])cc2)nc(O[C@H]2[C@@H](O)C([2H])([2H])OC([2H])([2H])C2([2H])[2H])c1C(F)(F)F]

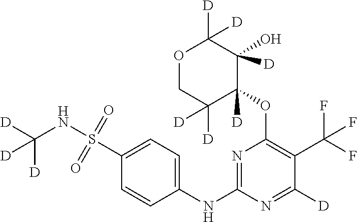 [SMILES: [2H]c1nc(Nc2ccc(S(=O)(=O)NC([2H])([2H])[2H])cc2)nc(O[C@]2([2H])C([2H])([2H])COC([2H])([2H])[C@]2([2H])O)c1C(F)(F)F]

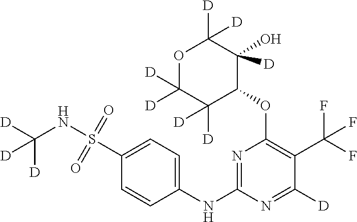 [SMILES: [2H]c1nc(Nc2ccc(S(=O)(=O)NC([2H])([2H])[2H])cc2)nc(O[C@@H]2C([2H])([2H])C([2H])([2H])OC([2H])([2H])[C@]2([2H])O)c1C(F)(F)F]

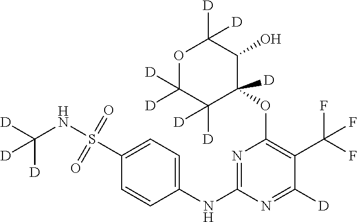 [SMILES: [2H]c1nc(Nc2ccc(S(=O)(=O)NC([2H])([2H])[2H])cc2)nc(O[C@@]2([2H])[C@@H](O)C([2H])([2H])OC([2H])([2H])C2([2H])[2H])c1C(F)(F)F]

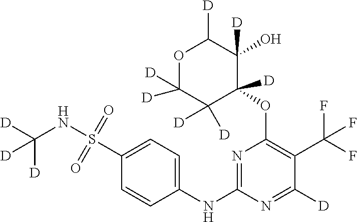 [SMILES: [2H]c1nc(Nc2ccc(S(=O)(=O)NC([2H])([2H])[2H])cc2)nc(O[C@]2([2H])C([2H])([2H])C([2H])([2H])OC([2H])[C@]2([2H])O)c1C(F)(F)F]

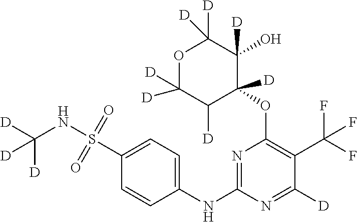 [SMILES: [2H]c1nc(Nc2ccc(S(=O)(=O)NC([2H])([2H])[2H])cc2)nc(O[C@]2([2H])C([2H])C([2H])([2H])OC([2H])([2H])[C@]2([2H])O)c1C(F)(F)F]

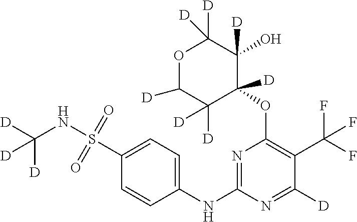 [SMILES: [2H]c1nc(Nc2ccc(S(=O)(=O)NC([2H])([2H])[2H])cc2)nc(O[C@]2([2H])C([2H])([2H])C([2H])OC([2H])([2H])[C@]2([2H])O)c1C(F)(F)F]

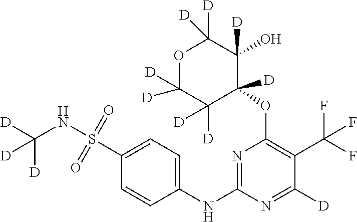 [SMILES: [2H]c1nc(Nc2ccc(S(=O)(=O)NC([2H])([2H])[2H])cc2)nc(O[C@]2([2H])C([2H])([2H])C([2H])([2H])OC([2H])([2H])[C@]2([2H])O)c1C(F)(F)F]

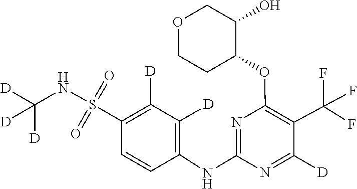 [SMILES: [2H]c1nc(Nc2ccc(S(=O)(=O)NC([2H])([2H])[2H])c([2H])c2[2H])nc(O[C@@H]2CCOC[C@@H]2O)c1C(F)(F)F]

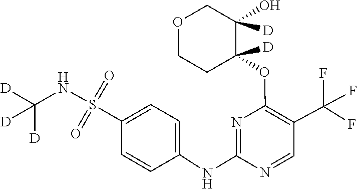 [SMILES: [2H]C([2H])([2H])NS(=O)(=O)c1ccc(Nc2ncc(C(F)(F)F)c(O[C@]3([2H])CCOC[C@]3([2H])O)n2)cc1]

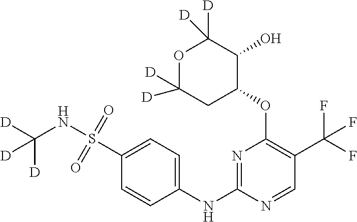 [SMILES: [2H]C([2H])([2H])NS(=O)(=O)c1ccc(Nc2ncc(C(F)(F)F)c(O[C@@H]3CC([2H])([2H])OC([2H])([2H])[C@@H]3O)n2)cc1]

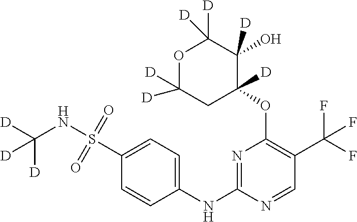 [SMILES: [2H]C([2H])([2H])NS(=O)(=O)c1ccc(Nc2ncc(C(F)(F)F)c(O[C@]3([2H])CC([2H])([2H])OC([2H])([2H])[C@]3([2H])O)n2)cc1]

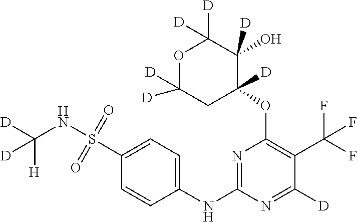 [SMILES: [2H]c1nc(Nc2ccc(S(=O)(=O)NC([2H])[2H])cc2)nc(O[C@]2([2H])CC([2H])([2H])OC([2H])([2H])[C@]2([2H])O)c1C(F)(F)F]

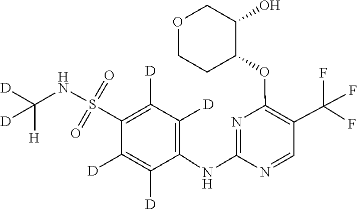 [SMILES: [2H]c1c([2H])c(S(=O)(=O)NC([2H])[2H])c([2H])c([2H])c1Nc1ncc(C(F)(F)F)c(O[C@@H]2CCOC[C@@H]2O)n1]

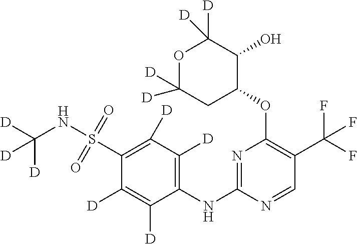 [SMILES: [2H]c1c([2H])c(S(=O)(=O)NC([2H])([2H])[2H])c([2H])c([2H])c1Nc1ncc(C(F)(F)F)c(O[C@@H]2CC([2H])([2H])OC([2H])([2H])[C@@H]2O)n1]

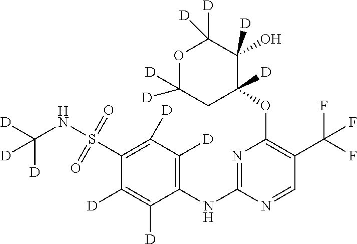 [SMILES: [2H]c1c([2H])c(S(=O)(=O)NC([2H])([2H])[2H])c([2H])c([2H])c1Nc1ncc(C(F)(F)F)c(O[C@]2([2H])CC([2H])([2H])OC([2H])([2H])[C@]2([2H])O)n1]

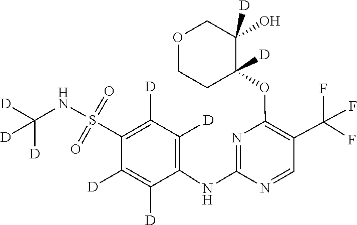 [SMILES: [2H]c1c([2H])c(S(=O)(=O)NC([2H])([2H])[2H])c([2H])c([2H])c1Nc1ncc(C(F)(F)F)c(O[C@]2([2H])CCOC[C@]2([2H])O)n1]

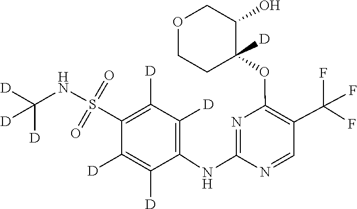 [SMILES: [2H]c1c([2H])c(S(=O)(=O)NC([2H])([2H])[2H])c([2H])c([2H])c1Nc1ncc(C(F)(F)F)c(O[C@]2([2H])CCOC[C@@H]2O)n1]

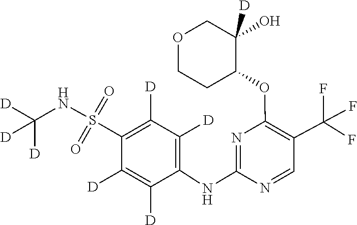 [SMILES: [2H]c1c([2H])c(S(=O)(=O)NC([2H])([2H])[2H])c([2H])c([2H])c1Nc1ncc(C(F)(F)F)c(O[C@@H]2CCOC[C@]2([2H])O)n1]

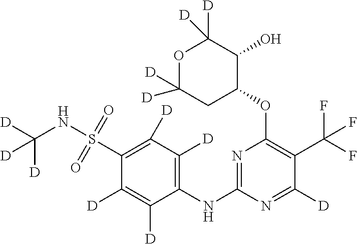 [SMILES: [2H]c1nc(Nc2c([2H])c([2H])c(S(=O)(=O)NC([2H])([2H])[2H])c([2H])c2[2H])nc(O[C@@H]2CC([2H])([2H])OC([2H])([2H])[C@@H]2O)c1C(F)(F)F]

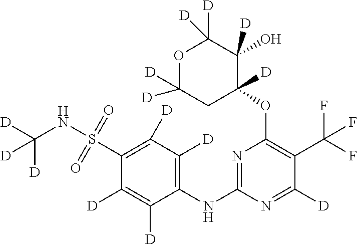 [SMILES: [2H]c1nc(Nc2c([2H])c([2H])c(S(=O)(=O)NC([2H])([2H])[2H])c([2H])c2[2H])nc(O[C@]2([2H])CC([2H])([2H])OC([2H])([2H])[C@]2([2H])O)c1C(F)(F)F]

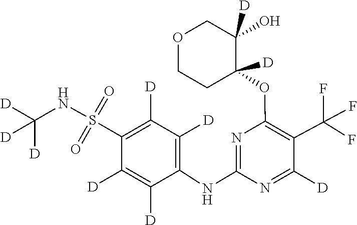 [SMILES: [2H]c1nc(Nc2c([2H])c([2H])c(S(=O)(=O)NC([2H])([2H])[2H])c([2H])c2[2H])nc(O[C@]2([2H])CCOC[C@]2([2H])O)c1C(F)(F)F]

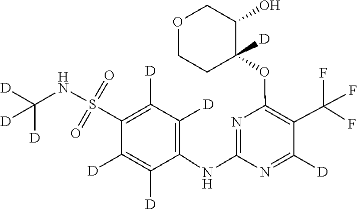 [SMILES: [2H]c1nc(Nc2c([2H])c([2H])c(S(=O)(=O)NC([2H])([2H])[2H])c([2H])c2[2H])nc(O[C@]2([2H])CCOC[C@@H]2O)c1C(F)(F)F]

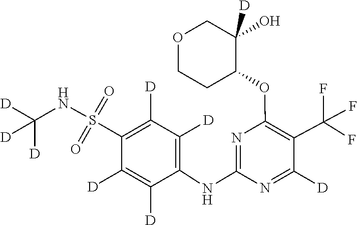 [SMILES: [2H]c1nc(Nc2c([2H])c([2H])c(S(=O)(=O)NC([2H])([2H])[2H])c([2H])c2[2H])nc(O[C@@H]2CCOC[C@]2([2H])O)c1C(F)(F)F]

TABLE 2
Deuterium enriched compounds of Formula (I):

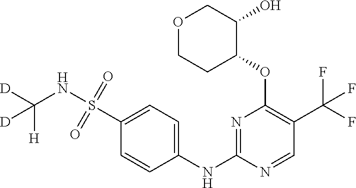 [SMILES: [2H]C([2H])NS(=O)(=O)c1ccc(Nc2ncc(C(F)(F)F)c(O[C@@H]3CCOC[C@@H]3O)n2)cc1]

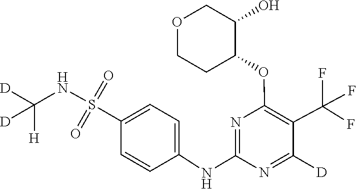 [SMILES: [2H]c1nc(Nc2ccc(S(=O)(=O)NC([2H])[2H])cc2)nc(O[C@@H]2CCOC[C@@H]2O)c1C(F)(F)F]

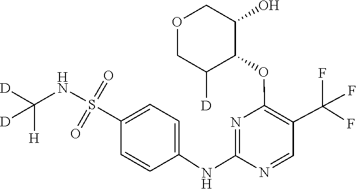 [SMILES: [2H]C([2H])NS(=O)(=O)c1ccc(Nc2ncc(C(F)(F)F)c(O[C@@H]3C([2H])COC[C@@H]3O)n2)cc1]

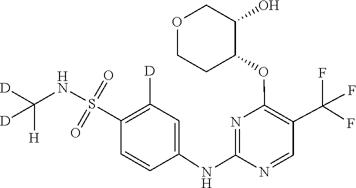 [SMILES: [2H]c1cc(Nc2ncc(C(F)(F)F)c(O[C@@H]3CCOC[C@@H]3O)n2)ccc1S(=O)(=O)NC([2H])[2H]]

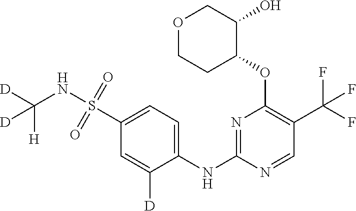 [SMILES: [2H]c1cc(S(=O)(=O)NC([2H])[2H])ccc1Nc1ncc(C(F)(F)F)c(O[C@@H]2CCOC[C@@H]2O)n1]

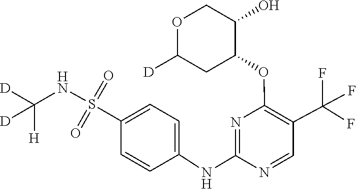 [SMILES: [2H]C([2H])NS(=O)(=O)c1ccc(Nc2ncc(C(F)(F)F)c(O[C@@H]3CC([2H])OC[C@@H]3O)n2)cc1]

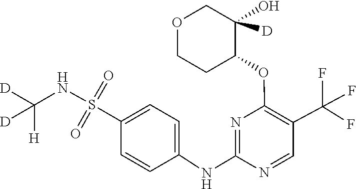 [SMILES: [2H]C([2H])NS(=O)(=O)c1ccc(Nc2ncc(C(F)(F)F)c(O[C@@H]3CCOC[C@]3([2H])O)n2)cc1]

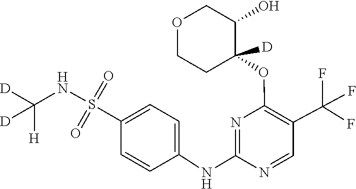 [SMILES: [2H]C([2H])NS(=O)(=O)c1ccc(Nc2ncc(C(F)(F)F)c(O[C@]3([2H])CCOC[C@@H]3O)n2)cc1]

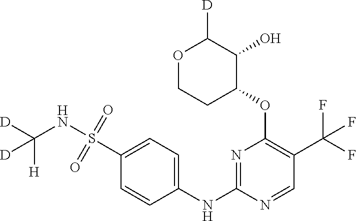 [SMILES: [2H]C([2H])NS(=O)(=O)c1ccc(Nc2ncc(C(F)(F)F)c(O[C@@H]3CCOC([2H])[C@@H]3O)n2)cc1]

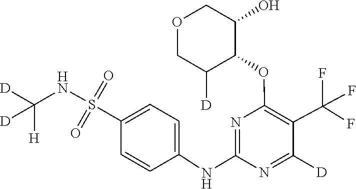 [SMILES: [2H]c1nc(Nc2ccc(S(=O)(=O)NC([2H])[2H])cc2)nc(O[C@@H]2C([2H])COC[C@@H]2O)c1C(F)(F)F]

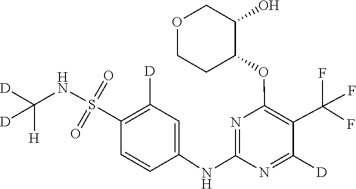 [SMILES: [2H]c1cc(Nc2nc([2H])c(C(F)(F)F)c(O[C@@H]3CCOC[C@@H]3O)n2)ccc1S(=O)(=O)NC([2H])[2H]]

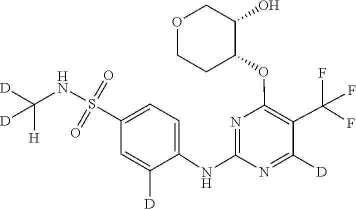 [SMILES: [2H]c1cc(S(=O)(=O)NC([2H])[2H])ccc1Nc1nc([2H])c(C(F)(F)F)c(O[C@@H]2CCOC[C@@H]2O)n1]

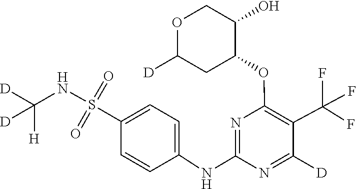 [SMILES: [2H]c1nc(Nc2ccc(S(=O)(=O)NC([2H])[2H])cc2)nc(O[C@@H]2CC([2H])OC[C@@H]2O)c1C(F)(F)F]

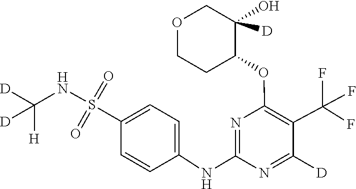 [SMILES: [2H]c1nc(Nc2ccc(S(=O)(=O)NC([2H])[2H])cc2)nc(O[C@@H]2CCOC[C@]2([2H])O)c1C(F)(F)F]

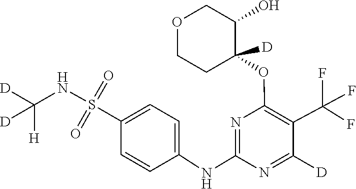 [SMILES: [2H]c1nc(Nc2ccc(S(=O)(=O)NC([2H])[2H])cc2)nc(O[C@]2([2H])CCOC[C@@H]2O)c1C(F)(F)F]

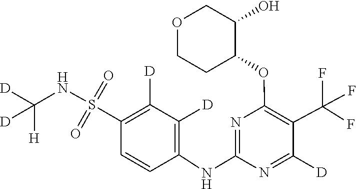 [SMILES: [2H]c1nc(Nc2ccc(S(=O)(=O)NC([2H])[2H])c([2H])c2[2H])nc(O[C@@H]2CCOC[C@@H]2O)c1C(F)(F)F]

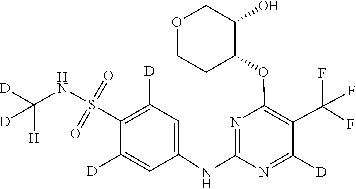 [SMILES: [2H]c1cc(Nc2nc([2H])c(C(F)(F)F)c(O[C@@H]3CCOC[C@@H]3O)n2)cc([2H])c1S(=O)(=O)NC([2H])[2H]]

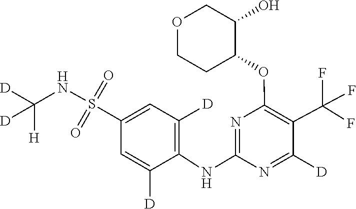 [SMILES: [2H]c1cc(S(=O)(=O)NC([2H])[2H])cc([2H])c1Nc1nc([2H])c(C(F)(F)F)c(O[C@@H]2CCOC[C@@H]2O)n1]

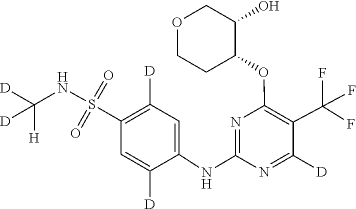 [SMILES: [2H]c1cc(S(=O)(=O)NC([2H])[2H])c([2H])cc1Nc1nc([2H])c(C(F)(F)F)c(O[C@@H]2CCOC[C@@H]2O)n1]

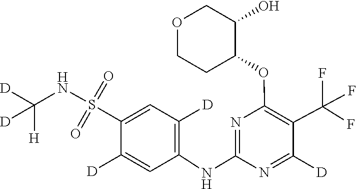 [SMILES: [2H]c1cc(S(=O)(=O)NC([2H])[2H])c([2H])cc1Nc1nc([2H])c(C(F)(F)F)c(O[C@@H]2CCOC[C@@H]2O)n1]

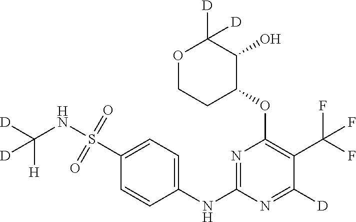 [SMILES: [2H]c1nc(Nc2ccc(S(=O)(=O)NC([2H])[2H])cc2)nc(O[C@@H]2CCOC([2H])([2H])[C@@H]2O)c1C(F)(F)F]

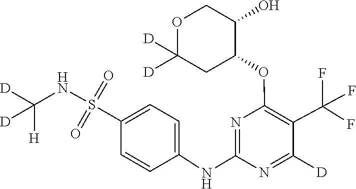 [SMILES: [2H]c1nc(Nc2ccc(S(=O)(=O)NC([2H])[2H])cc2)nc(O[C@@H]2CC([2H])([2H])OC[C@@H]2O)c1C(F)(F)F]

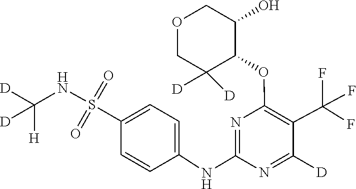 [SMILES: [2H]c1nc(Nc2ccc(S(=O)(=O)NC([2H])[2H])cc2)nc(O[C@H]2[C@@H](O)COCC2([2H])[2H])c1C(F)(F)F]

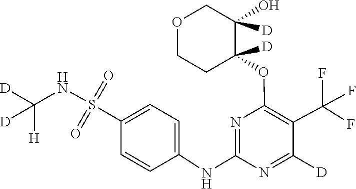 [SMILES: [2H]c1nc(Nc2ccc(S(=O)(=O)NC([2H])[2H])cc2)nc(O[C@]2([2H])CCOC[C@]2([2H])O)c1C(F)(F)F]

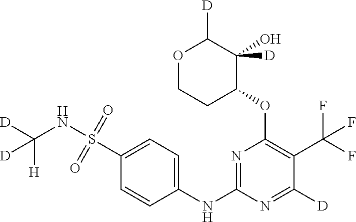 [SMILES: [2H]c1nc(Nc2ccc(S(=O)(=O)NC([2H])[2H])cc2)nc(O[C@@H]2CCOC([2H])[C@]2([2H])O)c1C(F)(F)F]

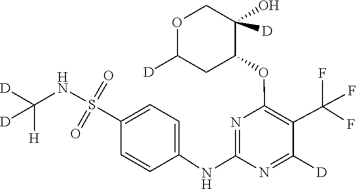 [SMILES: [2H]c1nc(Nc2ccc(S(=O)(=O)NC([2H])[2H])cc2)nc(O[C@@H]2CC([2H])OC[C@]2([2H])O)c1C(F)(F)F]

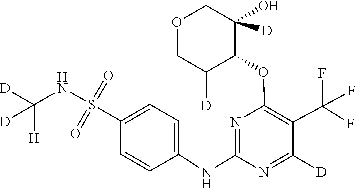 [SMILES: [2H]c1nc(Nc2ccc(S(=O)(=O)NC([2H])[2H])cc2)nc(O[C@@H]2C([2H])COC[C@]2([2H])O)c1C(F)(F)F]

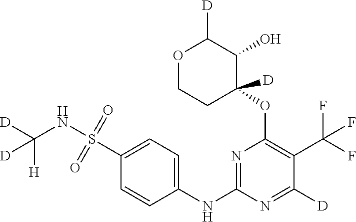 [SMILES: [2H]c1nc(Nc2ccc(S(=O)(=O)NC([2H])[2H])cc2)nc(O[C@]2([2H])CCOC([2H])[C@@H]2O)c1C(F)(F)F]

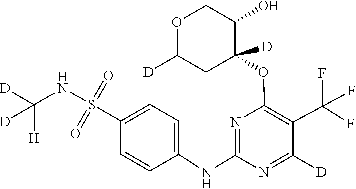 [SMILES: [2H]c1nc(Nc2ccc(S(=O)(=O)NC([2H])[2H])cc2)nc(O[C@]2([2H])CC([2H])OC[C@@H]2O)c1C(F)(F)F]

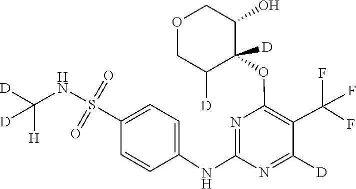 [SMILES: [2H]c1nc(Nc2ccc(S(=O)(=O)NC([2H])[2H])cc2)nc(O[C@]2([2H])C([2H])COC[C@@H]2O)c1C(F)(F)F]

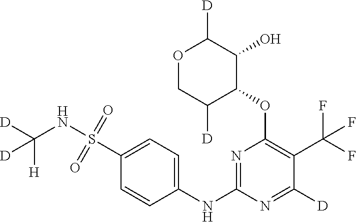 [SMILES: [2H]c1nc(Nc2ccc(S(=O)(=O)NC([2H])[2H])cc2)nc(O[C@@H]2C([2H])COC([2H])[C@@H]2O)c1C(F)(F)F]

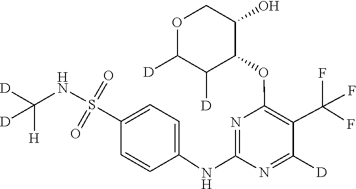 [SMILES: [2H]c1nc(Nc2ccc(S(=O)(=O)NC([2H])[2H])cc2)nc(O[C@@H]2C([2H])C([2H])OC[C@@H]2O)c1C(F)(F)F]

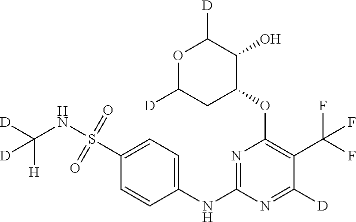 [SMILES: [2H]c1nc(Nc2ccc(S(=O)(=O)NC([2H])[2H])cc2)nc(O[C@@H]2CC([2H])OC([2H])[C@@H]2O)c1C(F)(F)F]

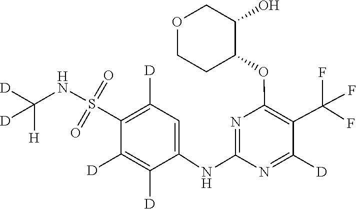 [SMILES: [2H]c1cc(Nc2nc([2H])c(C(F)(F)F)c(O[C@@H]3CCOC[C@@H]3O)n2)c([2H])c([2H])c1S(=O)(=O)NC([2H])[2H]]

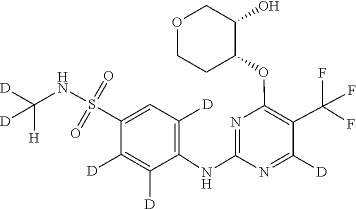 [SMILES: [2H]c1cc(S(=O)(=O)NC([2H])[2H])c([2H])c([2H])c1Nc1nc([2H])c(C(F)(F)F)c(O[C@@H]2CCOC[C@@H]2O)n1]

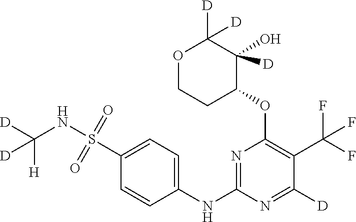 [SMILES: [2H]c1nc(Nc2ccc(S(=O)(=O)NC([2H])[2H])cc2)nc(O[C@@H]2CCOC([2H])([2H])[C@]2([2H])O)c1C(F)(F)F]

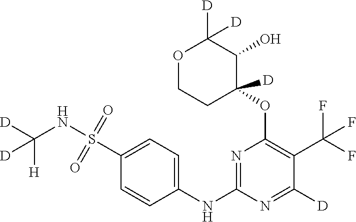 [SMILES: [2H]c1nc(Nc2ccc(S(=O)(=O)NC([2H])[2H])cc2)nc(O[C@]2([2H])CCOC([2H])([2H])[C@@H]2O)c1C(F)(F)F]

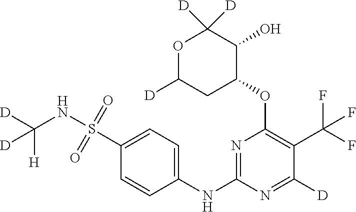 [SMILES: [2H]c1nc(Nc2ccc(S(=O)(=O)NC([2H])[2H])cc2)nc(O[C@@H]2CC([2H])OC([2H])([2H])[C@@H]2O)c1C(F)(F)F]

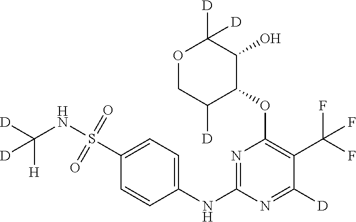 [SMILES: [2H]c1nc(Nc2ccc(S(=O)(=O)NC([2H])[2H])cc2)nc(O[C@@H]2C([2H])COC([2H])([2H])[C@@H]2O)c1C(F)(F)F]

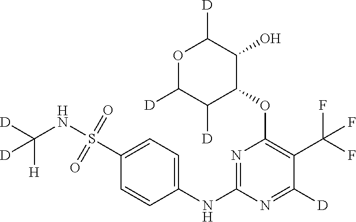 [SMILES: [2H]c1nc(Nc2ccc(S(=O)(=O)NC([2H])[2H])cc2)nc(O[C@@H]2C([2H])C([2H])OC([2H])[C@@H]2O)c1C(F)(F)F]

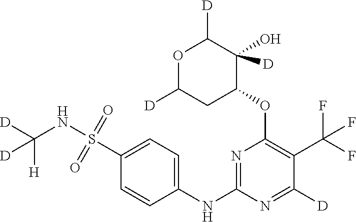 [SMILES: [2H]c1nc(Nc2ccc(S(=O)(=O)NC([2H])[2H])cc2)nc(O[C@@H]2CC([2H])OC([2H])[C@]2([2H])O)c1C(F)(F)F]

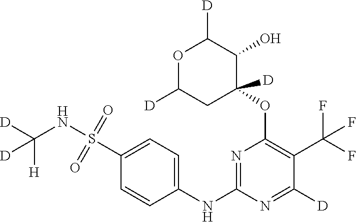 [SMILES: [2H]c1nc(Nc2ccc(S(=O)(=O)NC([2H])[2H])cc2)nc(O[C@]2([2H])CC([2H])OC([2H])[C@@H]2O)c1C(F)(F)F]

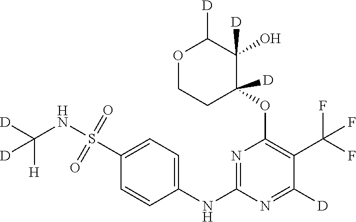 [SMILES: [2H]c1nc(Nc2ccc(S(=O)(=O)NC([2H])[2H])cc2)nc(O[C@]2([2H])CCOC([2H])[C@]2([2H])O)c1C(F)(F)F]

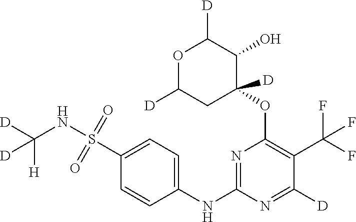 [SMILES: [2H]c1nc(Nc2ccc(S(=O)(=O)NC([2H])[2H])cc2)nc(O[C@]2([2H])CC([2H])OC([2H])[C@@H]2O)c1C(F)(F)F]

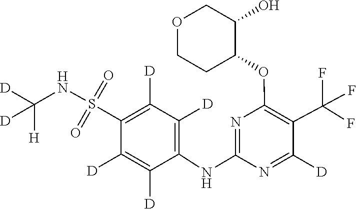 [SMILES: [2H]c1nc(Nc2c([2H])c([2H])c(S(=O)(=O)NC([2H])[2H])c([2H])c2[2H])nc(O[C@@H]2CCOC[C@@H]2O)c1C(F)(F)F]

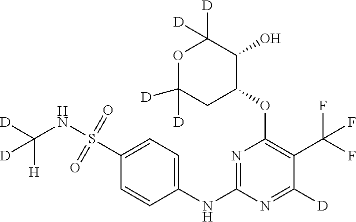 [SMILES: [2H]c1nc(Nc2ccc(S(=O)(=O)NC([2H])[2H])cc2)nc(O[C@@H]2CC([2H])([2H])OC([2H])([2H])[C@@H]2O)c1C(F)(F)F]

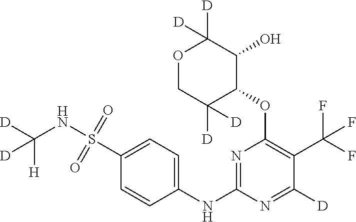 [SMILES: [2H]c1nc(Nc2ccc(S(=O)(=O)NC([2H])[2H])cc2)nc(O[C@H]2[C@@H](O)C([2H])([2H])OCC2([2H])[2H])c1C(F)(F)F]

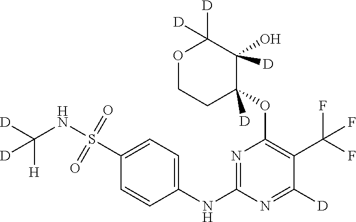 [SMILES: [2H]c1nc(Nc2ccc(S(=O)(=O)NC([2H])[2H])cc2)nc(O[C@]2([2H])CCOC([2H])([2H])[C@]2([2H])O)c1C(F)(F)F]

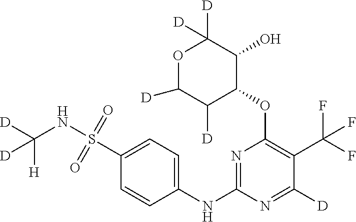 [SMILES: [2H]c1nc(Nc2ccc(S(=O)(=O)NC([2H])[2H])cc2)nc(O[C@@H]2C([2H])C([2H])OC([2H])([2H])[C@@H]2O)c1C(F)(F)F]

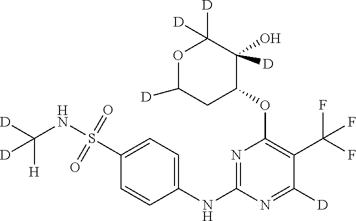 [SMILES: [2H]c1nc(Nc2ccc(S(=O)(=O)NC([2H])[2H])cc2)nc(O[C@@H]2CC([2H])OC([2H])([2H])[C@]2([2H])O)c1C(F)(F)F]

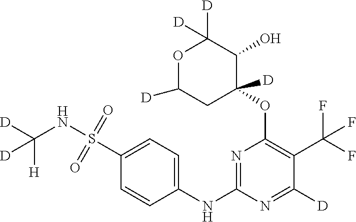 [SMILES: [2H]c1nc(Nc2ccc(S(=O)(=O)NC([2H])[2H])cc2)nc(O[C@]2([2H])CC([2H])OC([2H])([2H])[C@@H]2O)c1C(F)(F)F]

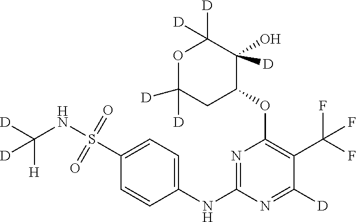 [SMILES: [2H]c1nc(Nc2ccc(S(=O)(=O)NC([2H])[2H])cc2)nc(O[C@@H]2CC([2H])([2H])OC([2H])([2H])[C@]2([2H])O)c1C(F)(F)F]

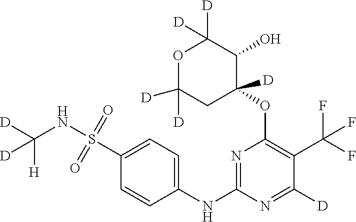 [SMILES: [2H]c1nc(Nc2ccc(S(=O)(=O)NC([2H])[2H])cc2)nc(O[C@]2([2H])CC([2H])([2H])OC([2H])([2H])[C@@H]2O)c1C(F)(F)F]

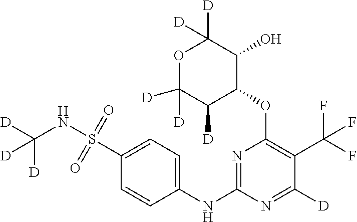 [SMILES: [2H]c1nc(Nc2ccc(S(=O)(=O)NC([2H])([2H])[2H])cc2)nc(O[C@@H]2[C@@H]([2H])C([2H])([2H])OC([2H])([2H])[C@@H]2O)c1C(F)(F)F]

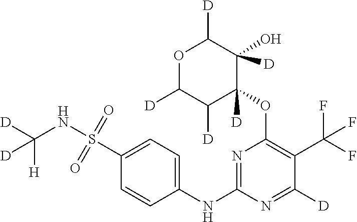 [SMILES: [2H]c1nc(Nc2ccc(S(=O)(=O)NC([2H])[2H])cc2)nc(O[C@]2([2H])C([2H])C([2H])OC([2H])[C@]2([2H])O)c1C(F)(F)F]

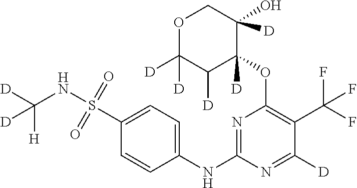 [SMILES: [2H]c1nc(Nc2ccc(S(=O)(=O)NC([2H])[2H])cc2)nc(O[C@]2([2H])C([2H])C([2H])([2H])OC[C@]2([2H])O)c1C(F)(F)F]

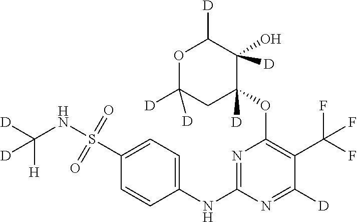 [SMILES: [2H]c1nc(Nc2ccc(S(=O)(=O)NC([2H])[2H])cc2)nc(O[C@]2([2H])CC([2H])([2H])OC([2H])[C@]2([2H])O)c1C(F)(F)F]

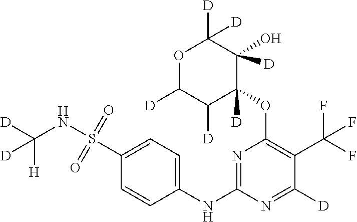 [SMILES: [2H]c1nc(Nc2ccc(S(=O)(=O)NC([2H])[2H])cc2)nc(O[C@]2([2H])C([2H])C([2H])OC([2H])([2H])[C@]2([2H])O)c1C(F)(F)F]

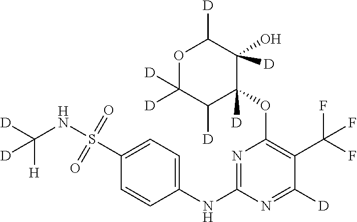 [SMILES: [2H]c1nc(Nc2ccc(S(=O)(=O)NC([2H])[2H])cc2)nc(O[C@]2([2H])C([2H])C([2H])([2H])OC([2H])[C@]2([2H])O)c1C(F)(F)F]

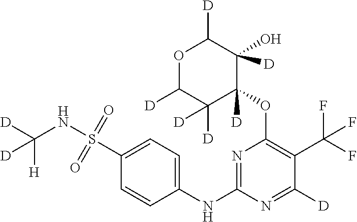 [SMILES: [2H]c1nc(Nc2ccc(S(=O)(=O)NC([2H])[2H])cc2)nc(O[C@]2([2H])C([2H])([2H])C([2H])OC([2H])[C@]2([2H])O)c1C(F)(F)F]

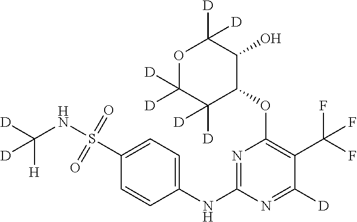 [SMILES: [2H]c1nc(Nc2ccc(S(=O)(=O)NC([2H])[2H])cc2)nc(O[C@H]2[C@@H](O)C([2H])([2H])OC([2H])([2H])C2([2H])[2H])c1C(F)(F)F]

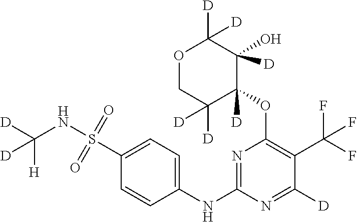 [SMILES: [2H]c1nc(Nc2ccc(S(=O)(=O)NC([2H])[2H])cc2)nc(O[C@]2([2H])C([2H])([2H])COC([2H])([2H])[C@]2([2H])O)c1C(F)(F)F]

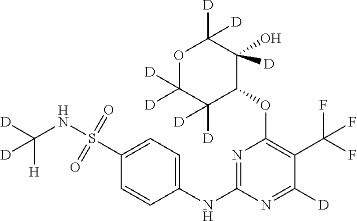 [SMILES: [2H]c1nc(Nc2ccc(S(=O)(=O)NC([2H])[2H])cc2)nc(O[C@@H]2C([2H])([2H])C([2H])([2H])OC([2H])([2H])[C@]2([2H])O)c1C(F)(F)F]

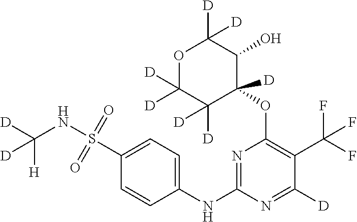 [SMILES: [2H]c1nc(Nc2ccc(S(=O)(=O)NC([2H])[2H])cc2)nc(O[C@@]2([2H])[C@@H](O)C([2H])([2H])OC([2H])([2H])C2([2H])[2H])c1C(F)(F)F]

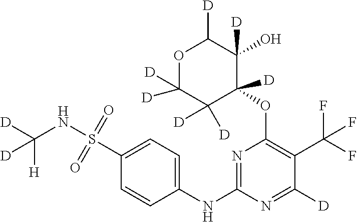 [SMILES: [2H]c1nc(Nc2ccc(S(=O)(=O)NC([2H])[2H])cc2)nc(O[C@]2([2H])C([2H])([2H])C([2H])([2H])OC([2H])[C@]2([2H])O)c1C(F)(F)F]

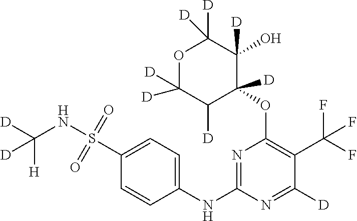 [SMILES: [2H]c1nc(Nc2ccc(S(=O)(=O)NC([2H])[2H])cc2)nc(O[C@]2([2H])C([2H])C([2H])([2H])OC([2H])([2H])[C@]2([2H])O)c1C(F)(F)F]

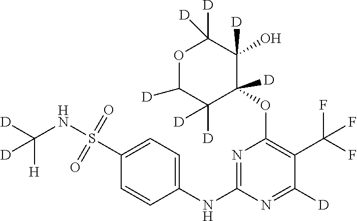 [SMILES: [2H]c1nc(Nc2ccc(S(=O)(=O)NC([2H])[2H])cc2)nc(O[C@]2([2H])C([2H])([2H])C([2H])OC([2H])([2H])[C@]2([2H])O)c1C(F)(F)F]

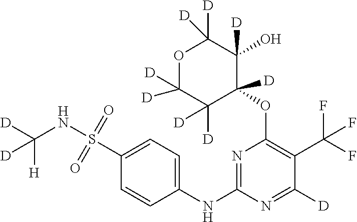 [SMILES: [2H]c1nc(Nc2ccc(S(=O)(=O)NC([2H])[2H])cc2)nc(O[C@]2([2H])C([2H])([2H])C([2H])([2H])OC([2H])([2H])[C@]2([2H])O)c1C(F)(F)F]

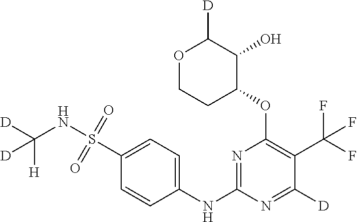 [SMILES: [2H]c1nc(Nc2ccc(S(=O)(=O)NC([2H])[2H])cc2)nc(O[C@@H]2CCOC([2H])[C@@H]2O)c1C(F)(F)F]

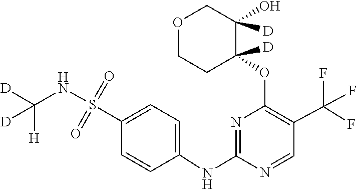 [SMILES: [2H]C([2H])NS(=O)(=O)c1ccc(Nc2ncc(C(F)(F)F)c(O[C@]3([2H])CCOC[C@]3([2H])O)n2)cc1]

TABLE 3
Deuterium enriched compounds of Formula (I):

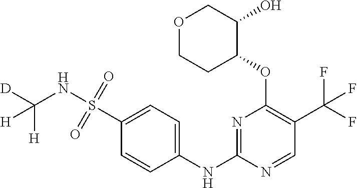 [SMILES: [2H]CNS(=O)(=O)c1ccc(Nc2ncc(C(F)(F)F)c(O[C@@H]3CCOC[C@@H]3O)n2)cc1]

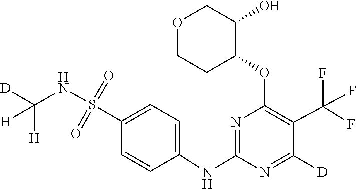 [SMILES: [2H]CNS(=O)(=O)c1ccc(Nc2nc([2H])c(C(F)(F)F)c(O[C@@H]3CCOC[C@@H]3O)n2)cc1]

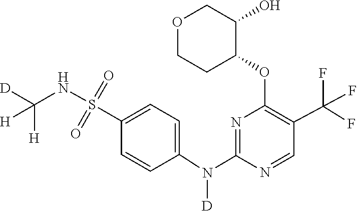

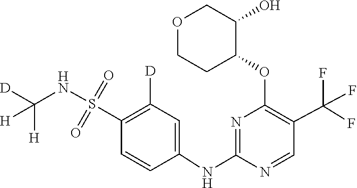 [SMILES: [2H]CNS(=O)(=O)c1ccc(Nc2ncc(C(F)(F)F)c(O[C@@H]3CCOC[C@@H]3O)n2)cc1[2H]]

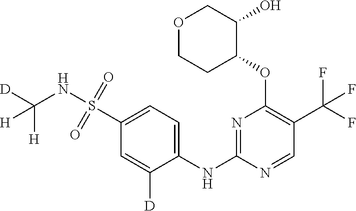 [SMILES: [2H]CNS(=O)(=O)c1ccc(Nc2ncc(C(F)(F)F)c(O[C@@H]3CCOC[C@@H]3O)n2)c([2H])c1]

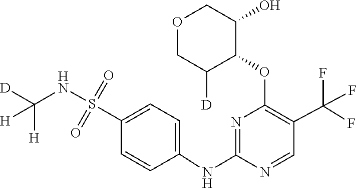 [SMILES: [2H]CNS(=O)(=O)c1ccc(Nc2ncc(C(F)(F)F)c(O[C@@H]3C([2H])COC[C@@H]3O)n2)cc1]

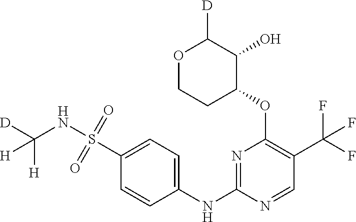 [SMILES: [2H]CNS(=O)(=O)c1ccc(Nc2ncc(C(F)(F)F)c(O[C@@H]3CCOC([2H])[C@@H]3O)n2)cc1]

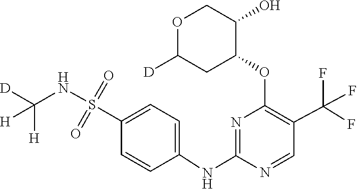 [SMILES: [2H]CNS(=O)(=O)c1ccc(Nc2ncc(C(F)(F)F)c(O[C@@H]3CC([2H])OC[C@@H]3O)n2)cc1]

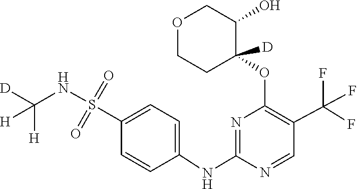 [SMILES: [2H]CNS(=O)(=O)c1ccc(Nc2ncc(C(F)(F)F)c(O[C@]3([2H])CCOC[C@@H]3O)n2)cc1]

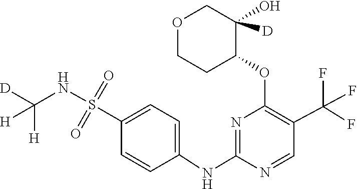 [SMILES: [2H]CNS(=O)(=O)c1ccc(Nc2ncc(C(F)(F)F)c(O[C@@H]3CCOC[C@]3([2H])O)n2)cc1]

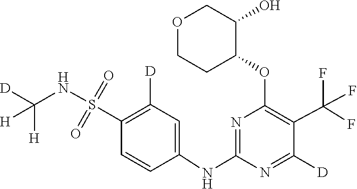 [SMILES: [2H]CNS(=O)(=O)c1ccc(Nc2nc([2H])c(C(F)(F)F)c(O[C@@H]3CCOC[C@@H]3O)n2)cc1[2H]]

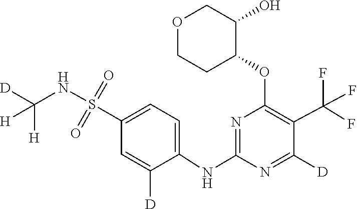 [SMILES: [2H]CNS(=O)(=O)c1ccc(Nc2nc([2H])c(C(F)(F)F)c(O[C@@H]3CCOC[C@@H]3O)n2)c([2H])c1]

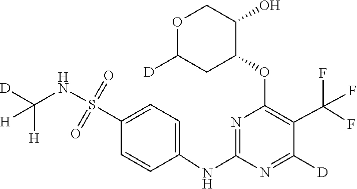 [SMILES: [2H]CNS(=O)(=O)c1ccc(Nc2nc([2H])c(C(F)(F)F)c(O[C@@H]3CC([2H])OC[C@@H]3O)n2)cc1]

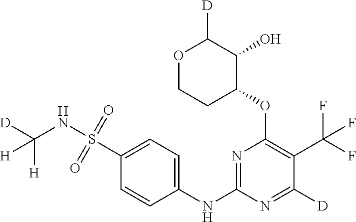 [SMILES: [2H]CNS(=O)(=O)c1ccc(Nc2nc([2H])c(C(F)(F)F)c(O[C@@H]3CCOC([2H])[C@@H]3O)n2)cc1]

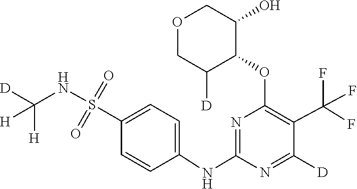 [SMILES: [2H]CNS(=O)(=O)c1ccc(Nc2nc([2H])c(C(F)(F)F)c(O[C@@H]3C([2H])COC[C@@H]3O)n2)cc1]

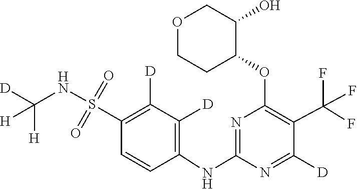 [SMILES: [2H]CNS(=O)(=O)c1ccc(Nc2nc([2H])c(C(F)(F)F)c(O[C@@H]3CCOC[C@@H]3O)n2)c([2H])c1[2H]]

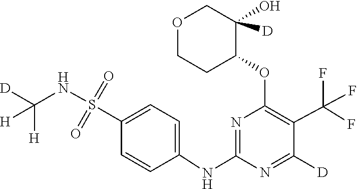 [SMILES: [2H]CNS(=O)(=O)c1ccc(Nc2nc([2H])c(C(F)(F)F)c(O[C@@H]3CCOC[C@]3([2H])O)n2)cc1]

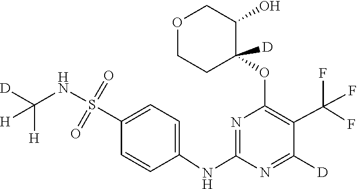 [SMILES: [2H]CNS(=O)(=O)c1ccc(Nc2nc([2H])c(C(F)(F)F)c(O[C@]3([2H])CCOC[C@@H]3O)n2)cc1]

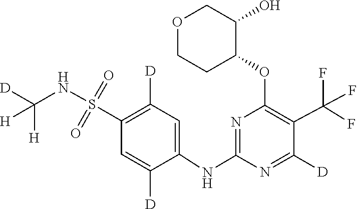 [SMILES: [2H]CNS(=O)(=O)c1cc([2H])c(Nc2nc([2H])c(C(F)(F)F)c(O[C@@H]3CCOC[C@@H]3O)n2)cc1[2H]]

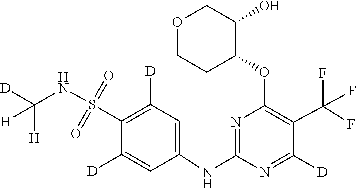 [SMILES: [2H]CNS(=O)(=O)c1c([2H])cc(Nc2nc([2H])c(C(F)(F)F)c(O[C@@H]3CCOC[C@@H]3O)n2)cc1[2H]]

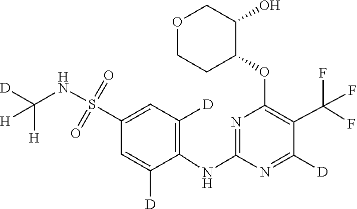 [SMILES: [2H]CNS(=O)(=O)c1cc([2H])c(Nc2nc([2H])c(C(F)(F)F)c(O[C@@H]3CCOC[C@@H]3O)n2)c([2H])c1]

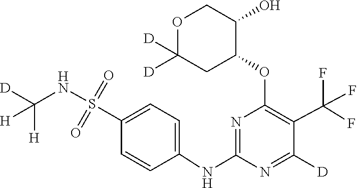 [SMILES: [2H]CNS(=O)(=O)c1ccc(Nc2nc([2H])c(C(F)(F)F)c(O[C@@H]3CC([2H])([2H])OC[C@@H]3O)n2)cc1]

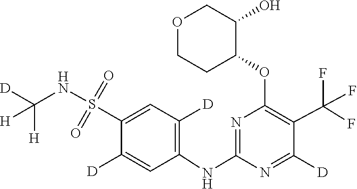 [SMILES: [2H]CNS(=O)(=O)c1cc([2H])c(Nc2nc([2H])c(C(F)(F)F)c(O[C@@H]3CCOC[C@@H]3O)n2)cc1[2H]]

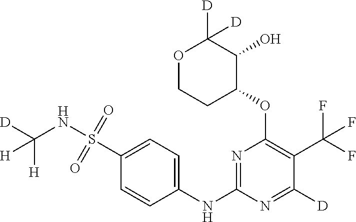 [SMILES: [2H]CNS(=O)(=O)c1ccc(Nc2nc([2H])c(C(F)(F)F)c(O[C@@H]3CCOC([2H])([2H])[C@@H]3O)n2)cc1]

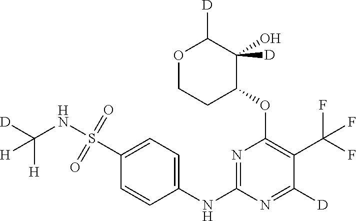 [SMILES: [2H]CNS(=O)(=O)c1ccc(Nc2nc([2H])c(C(F)(F)F)c(O[C@@H]3CCOC([2H])[C@]3([2H])O)n2)cc1]

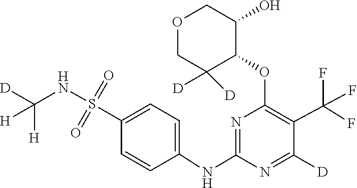 [SMILES: [2H]CNS(=O)(=O)c1ccc(Nc2nc([2H])c(C(F)(F)F)c(O[C@H]3[C@@H](O)COCC3([2H])[2H])n2)cc1]

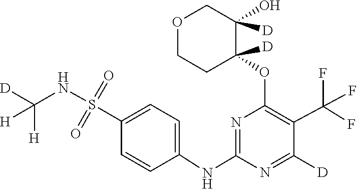 [SMILES: [2H]CNS(=O)(=O)c1ccc(Nc2nc([2H])c(C(F)(F)F)c(O[C@]3([2H])CCOC[C@]3([2H])O)n2)cc1]

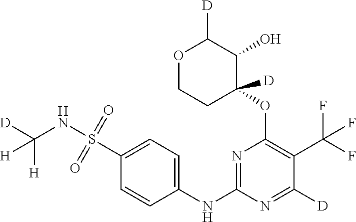 [SMILES: [2H]CNS(=O)(=O)c1ccc(Nc2nc([2H])c(C(F)(F)F)c(O[C@]3([2H])CCOC([2H])[C@@H]3O)n2)cc1]

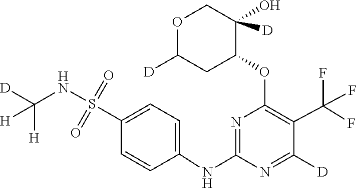 [SMILES: [2H]CNS(=O)(=O)c1ccc(Nc2nc([2H])c(C(F)(F)F)c(O[C@@H]3CC([2H])OC[C@]3([2H])O)n2)cc1]

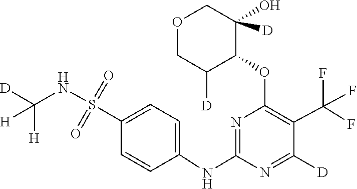 [SMILES: [2H]CNS(=O)(=O)c1ccc(Nc2nc([2H])c(C(F)(F)F)c(O[C@@H]3C([2H])COC[C@]3([2H])O)n2)cc1]

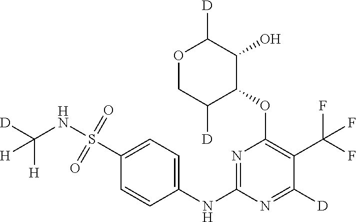 [SMILES: [2H]CNS(=O)(=O)c1ccc(Nc2nc([2H])c(C(F)(F)F)c(O[C@@H]3C([2H])COC([2H])[C@@H]3O)n2)cc1]

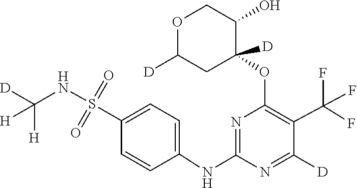 [SMILES: [2H]CNS(=O)(=O)c1ccc(Nc2nc([2H])c(C(F)(F)F)c(O[C@]3([2H])CC([2H])OC[C@@H]3O)n2)cc1]

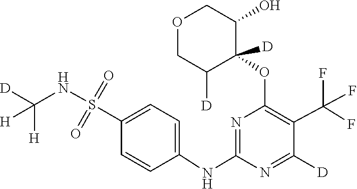 [SMILES: [2H]CNS(=O)(=O)c1ccc(Nc2nc([2H])c(C(F)(F)F)c(O[C@]3([2H])C([2H])COC[C@@H]3O)n2)cc1]

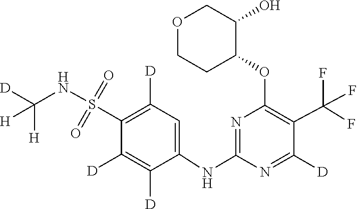 [SMILES: [2H]CNS(=O)(=O)c1c([2H])cc(Nc2nc([2H])c(C(F)(F)F)c(O[C@@H]3CCOC[C@@H]3O)n2)c([2H])c1[2H]]

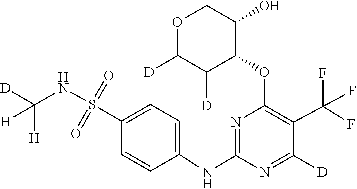 [SMILES: [2H]CNS(=O)(=O)c1ccc(Nc2nc([2H])c(C(F)(F)F)c(O[C@@H]3C([2H])C([2H])OC[C@@H]3O)n2)cc1]

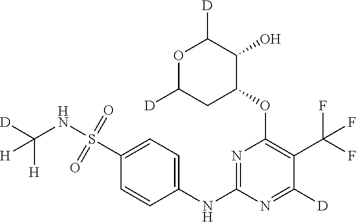 [SMILES: [2H]CNS(=O)(=O)c1ccc(Nc2nc([2H])c(C(F)(F)F)c(O[C@@H]3CC([2H])OC([2H])[C@@H]3O)n2)cc1]

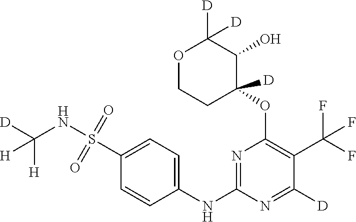 [SMILES: [2H]CNS(=O)(=O)c1ccc(Nc2nc([2H])c(C(F)(F)F)c(O[C@]3([2H])CCOC([2H])([2H])[C@@H]3O)n2)cc1]

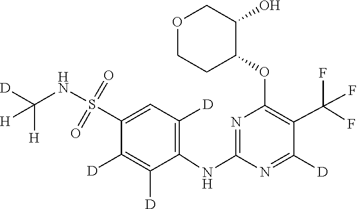 [SMILES: [2H]CNS(=O)(=O)c1cc([2H])c(Nc2nc([2H])c(C(F)(F)F)c(O[C@@H]3CCOC[C@@H]3O)n2)c([2H])c1[2H]]

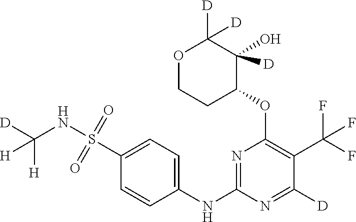 [SMILES: [2H]CNS(=O)(=O)c1ccc(Nc2nc([2H])c(C(F)(F)F)c(O[C@@H]3CCOC([2H])([2H])[C@]3([2H])O)n2)cc1]

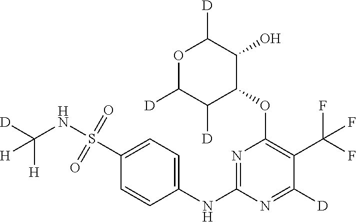 [SMILES: [2H]CNS(=O)(=O)c1ccc(Nc2nc([2H])c(C(F)(F)F)c(O[C@@H]3C([2H])C([2H])OC([2H])[C@@H]3O)n2)cc1]

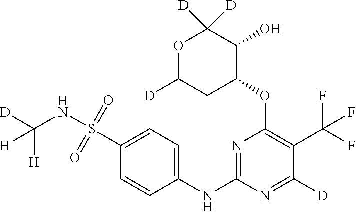 [SMILES: [2H]CNS(=O)(=O)c1ccc(Nc2nc([2H])c(C(F)(F)F)c(O[C@@H]3CC([2H])OC([2H])([2H])[C@@H]3O)n2)cc1]

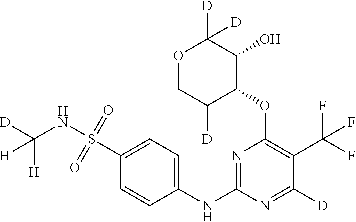 [SMILES: [2H]CNS(=O)(=O)c1ccc(Nc2nc([2H])c(C(F)(F)F)c(O[C@@H]3C([2H])COC([2H])([2H])[C@@H]3O)n2)cc1]

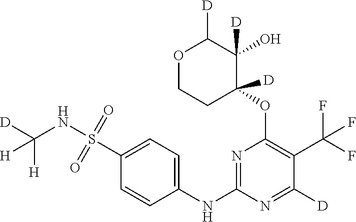 [SMILES: [2H]CNS(=O)(=O)c1ccc(Nc2nc([2H])c(C(F)(F)F)c(O[C@]3([2H])CCOC([2H])[C@]3([2H])O)n2)cc1]

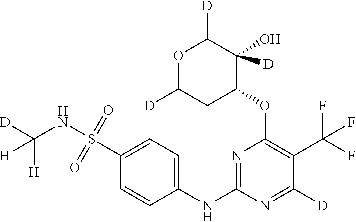 [SMILES: [2H]CNS(=O)(=O)c1ccc(Nc2nc([2H])c(C(F)(F)F)c(O[C@@H]3CC([2H])OC([2H])[C@]3([2H])O)n2)cc1]

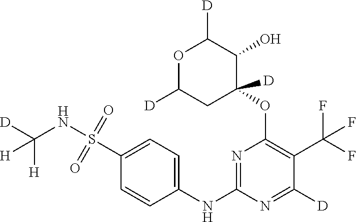 [SMILES: [2H]CNS(=O)(=O)c1ccc(Nc2nc([2H])c(C(F)(F)F)c(O[C@]3([2H])CC([2H])OC([2H])[C@@H]3O)n2)cc1]

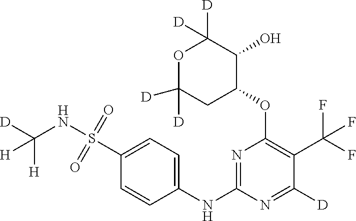 [SMILES: [2H]CNS(=O)(=O)c1ccc(Nc2nc([2H])c(C(F)(F)F)c(O[C@@H]3CC([2H])([2H])OC([2H])([2H])[C@@H]3O)n2)cc1]

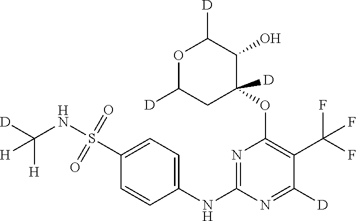 [SMILES: [2H]CNS(=O)(=O)c1ccc(Nc2nc([2H])c(C(F)(F)F)c(O[C@]3([2H])CC([2H])OC([2H])[C@@H]3O)n2)cc1]

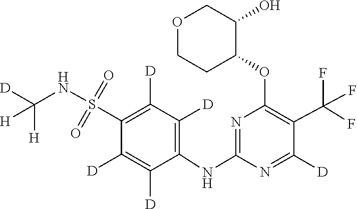 [SMILES: [2H]CNS(=O)(=O)c1c([2H])c([2H])c(Nc2nc([2H])c(C(F)(F)F)c(O[C@@H]3CCOC[C@@H]3O)n2)c([2H])c1[2H]]

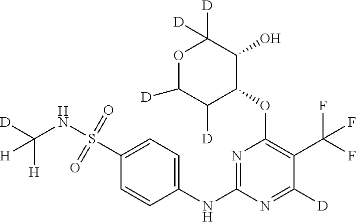 [SMILES: [2H]CNS(=O)(=O)c1ccc(Nc2nc([2H])c(C(F)(F)F)c(O[C@@H]3C([2H])C([2H])OC([2H])([2H])[C@@H]3O)n2)cc1]

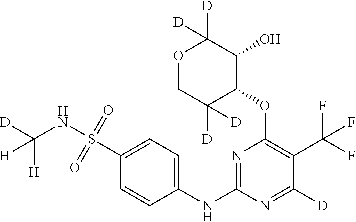 [SMILES: [2H]CNS(=O)(=O)c1ccc(Nc2nc([2H])c(C(F)(F)F)c(O[C@H]3[C@@H](O)C([2H])([2H])OCC3([2H])[2H])n2)cc1]

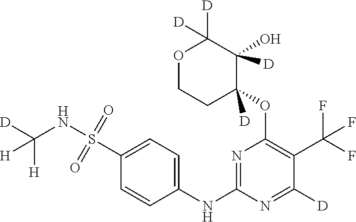 [SMILES: [2H]CNS(=O)(=O)c1ccc(Nc2nc([2H])c(C(F)(F)F)c(O[C@]3([2H])CCOC([2H])([2H])[C@]3([2H])O)n2)cc1]

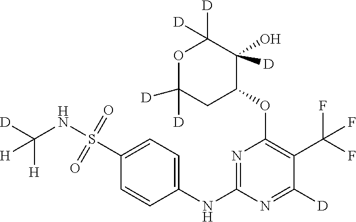 [SMILES: [2H]CNS(=O)(=O)c1ccc(Nc2nc([2H])c(C(F)(F)F)c(O[C@@H]3CC([2H])([2H])OC([2H])([2H])[C@]3([2H])O)n2)cc1]

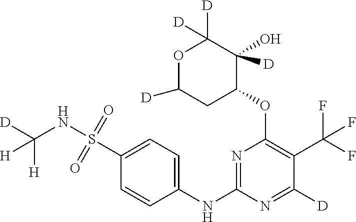 [SMILES: [2H]CNS(=O)(=O)c1ccc(Nc2nc([2H])c(C(F)(F)F)c(O[C@@H]3CC([2H])OC([2H])([2H])[C@]3([2H])O)n2)cc1]

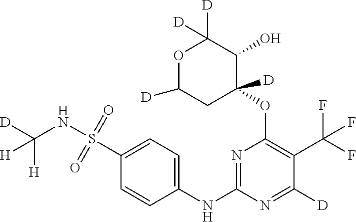 [SMILES: [2H]CNS(=O)(=O)c1ccc(Nc2nc([2H])c(C(F)(F)F)c(O[C@]3([2H])CC([2H])OC([2H])([2H])[C@@H]3O)n2)cc1]

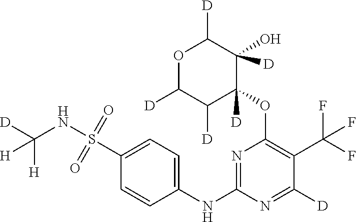 [SMILES: [2H]CNS(=O)(=O)c1ccc(Nc2nc([2H])c(C(F)(F)F)c(O[C@]3([2H])C([2H])C([2H])OC([2H])[C@]3([2H])O)n2)cc1]

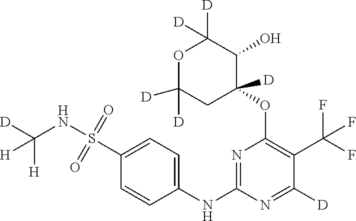 [SMILES: [2H]CNS(=O)(=O)c1ccc(Nc2nc([2H])c(C(F)(F)F)c(O[C@]3([2H])CC([2H])([2H])OC([2H])([2H])[C@@H]3O)n2)cc1]

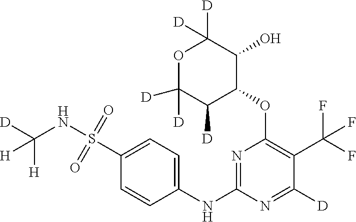 [SMILES: [2H]CNS(=O)(=O)c1ccc(Nc2nc([2H])c(C(F)(F)F)c(O[C@@H]3[C@@H]([2H])C([2H])([2H])OC([2H])([2H])[C@@H]3O)n2)cc1]

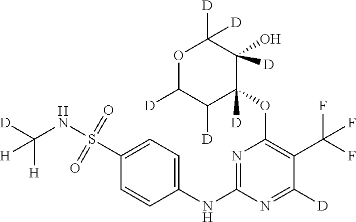 [SMILES: [2H]CNS(=O)(=O)c1ccc(Nc2nc([2H])c(C(F)(F)F)c(O[C@]3([2H])C([2H])C([2H])OC([2H])([2H])[C@]3([2H])O)n2)cc1]

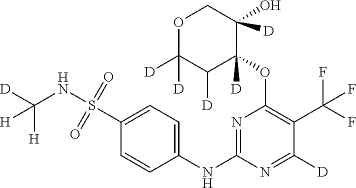 [SMILES: [2H]CNS(=O)(=O)c1ccc(Nc2nc([2H])c(C(F)(F)F)c(O[C@]3([2H])C([2H])C([2H])([2H])OC[C@]3([2H])O)n2)cc1]

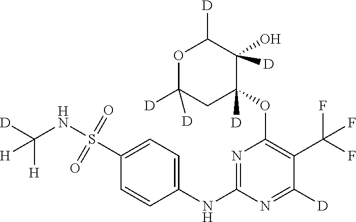 [SMILES: [2H]CNS(=O)(=O)c1ccc(Nc2nc([2H])c(C(F)(F)F)c(O[C@]3([2H])CC([2H])([2H])OC([2H])[C@]3([2H])O)n2)cc1]

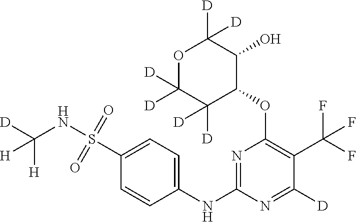 [SMILES: [2H]CNS(=O)(=O)c1ccc(Nc2nc([2H])c(C(F)(F)F)c(O[C@H]3[C@@H](O)C([2H])([2H])OC([2H])([2H])C3([2H])[2H])n2)cc1]

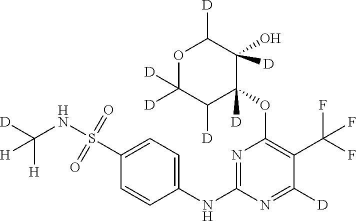 [SMILES: [2H]CNS(=O)(=O)c1ccc(Nc2nc([2H])c(C(F)(F)F)c(O[C@]3([2H])C([2H])C([2H])([2H])OC([2H])[C@]3([2H])O)n2)cc1]

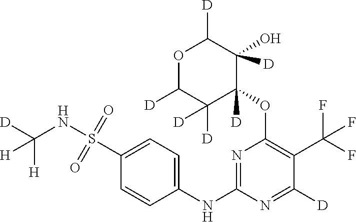 [SMILES: [2H]CNS(=O)(=O)c1ccc(Nc2nc([2H])c(C(F)(F)F)c(O[C@]3([2H])C([2H])([2H])C([2H])OC([2H])[C@]3([2H])O)n2)cc1]

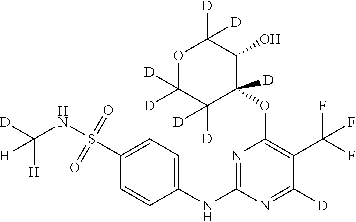 [SMILES: [2H]CNS(=O)(=O)c1ccc(Nc2nc([2H])c(C(F)(F)F)c(O[C@@]3([2H])[C@@H](O)C([2H])([2H])OC([2H])([2H])C3([2H])[2H])n2)cc1]

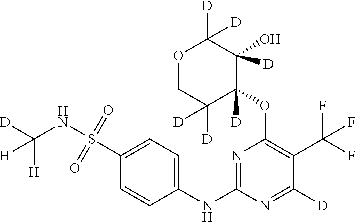 [SMILES: [2H]CNS(=O)(=O)c1ccc(Nc2nc([2H])c(C(F)(F)F)c(O[C@]3([2H])C([2H])([2H])COC([2H])([2H])[C@]3([2H])O)n2)cc1]

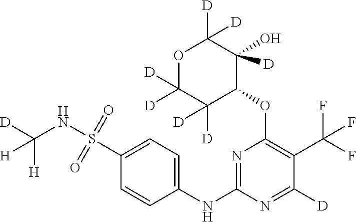 [SMILES: [2H]CNS(=O)(=O)c1ccc(Nc2nc([2H])c(C(F)(F)F)c(O[C@@H]3C([2H])([2H])C([2H])([2H])OC([2H])([2H])[C@]3([2H])O)n2)cc1]

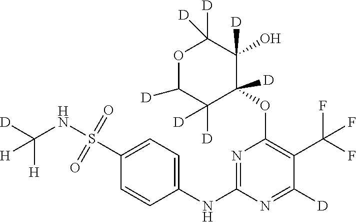 [SMILES: [2H]CNS(=O)(=O)c1ccc(Nc2nc([2H])c(C(F)(F)F)c(O[C@]3([2H])C([2H])([2H])C([2H])OC([2H])([2H])[C@]3([2H])O)n2)cc1]

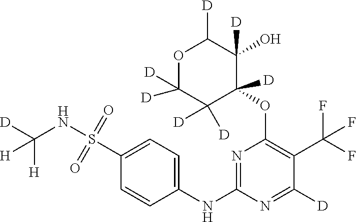 [SMILES: [2H]CNS(=O)(=O)c1ccc(Nc2nc([2H])c(C(F)(F)F)c(O[C@]3([2H])C([2H])([2H])C([2H])([2H])OC([2H])[C@]3([2H])O)n2)cc1]

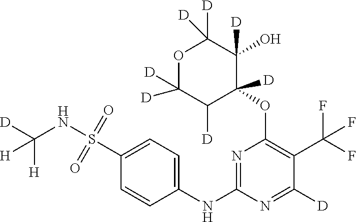 [SMILES: [2H]CNS(=O)(=O)c1ccc(Nc2nc([2H])c(C(F)(F)F)c(O[C@]3([2H])C([2H])C([2H])([2H])OC([2H])([2H])[C@]3([2H])O)n2)cc1]

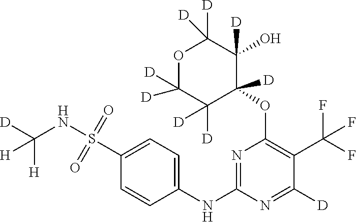 [SMILES: [2H]CNS(=O)(=O)c1ccc(Nc2nc([2H])c(C(F)(F)F)c(O[C@]3([2H])C([2H])([2H])C([2H])([2H])OC([2H])([2H])[C@]3([2H])O)n2)cc1]

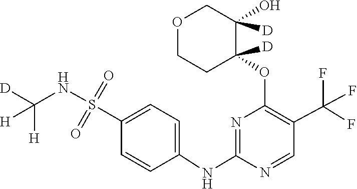 [SMILES: [2H]CNS(=O)(=O)c1ccc(Nc2ncc(C(F)(F)F)c(O[C@]3([2H])CCOC[C@]3([2H])O)n2)cc1]

TABLE 4
Deuterium enriched compounds of Formula (I):

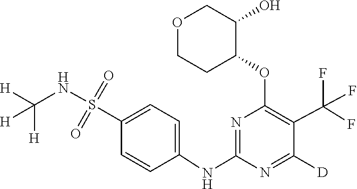 [SMILES: [2H]c1nc(Nc2ccc(S(=O)(=O)NC)cc2)nc(O[C@@H]2CCOC[C@@H]2O)c1C(F)(F)F]

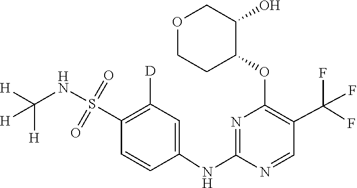 [SMILES: [2H]c1cc(Nc2ncc(C(F)(F)F)c(O[C@@H]3CCOC[C@@H]3O)n2)ccc1S(=O)(=O)NC]

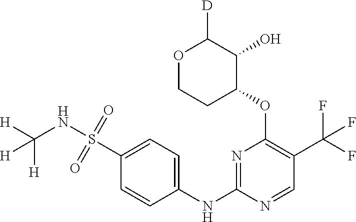 [SMILES: [2H]C1OCC[C@@H](Oc2nc(Nc3ccc(S(=O)(=O)NC)cc3)ncc2C(F)(F)F)[C@H]1O]

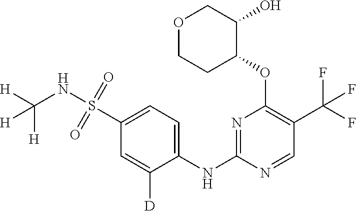 [SMILES: [2H]c1cc(S(=O)(=O)NC)ccc1Nc1ncc(C(F)(F)F)c(O[C@@H]2CCOC[C@@H]2O)n1]

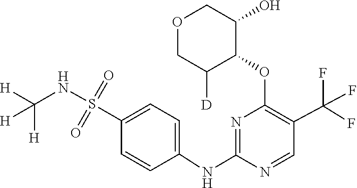 [SMILES: [2H]C1COC[C@H](O)[C@@H]1Oc1nc(Nc2ccc(S(=O)(=O)NC)cc2)ncc1C(F)(F)F]

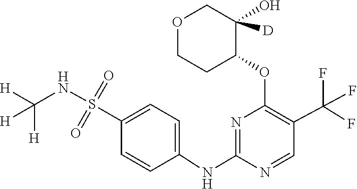 [SMILES: [2H][C@]1(O)COCC[C@H]1Oc1nc(Nc2ccc(S(=O)(=O)NC)cc2)ncc1C(F)(F)F]

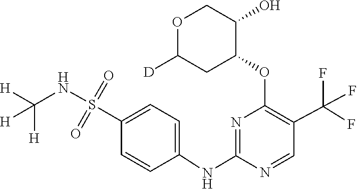 [SMILES: [2H]C1C[C@@H](Oc2nc(Nc3ccc(S(=O)(=O)NC)cc3)ncc2C(F)(F)F)[C@@H](O)CO1]

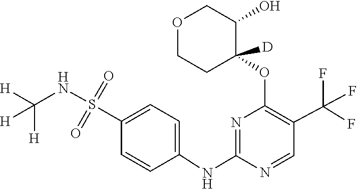 [SMILES: [2H][C@@]1(Oc2nc(Nc3ccc(S(=O)(=O)NC)cc3)ncc2C(F)(F)F)CCOC[C@@H]1O]

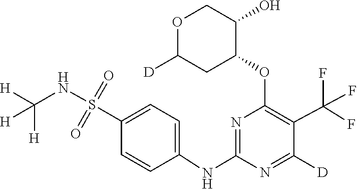 [SMILES: [2H]c1nc(Nc2ccc(S(=O)(=O)NC)cc2)nc(O[C@@H]2CC([2H])OC[C@@H]2O)c1C(F)(F)F]

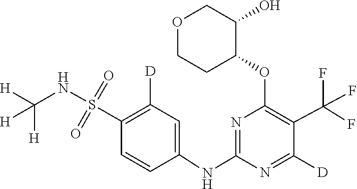 [SMILES: [2H]c1cc(Nc2nc([2H])c(C(F)(F)F)c(O[C@@H]3CCOC[C@@H]3O)n2)ccc1S(=O)(=O)NC]

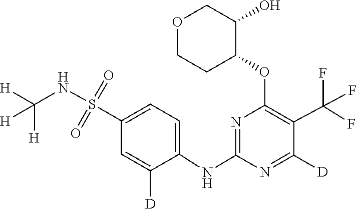 [SMILES: [2H]c1cc(S(=O)(=O)NC)ccc1Nc1nc([2H])c(C(F)(F)F)c(O[C@@H]2CCOC[C@@H]2O)n1]

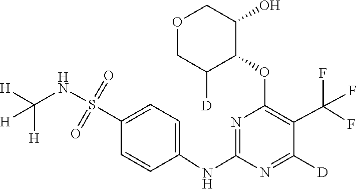 [SMILES: [2H]c1nc(Nc2ccc(S(=O)(=O)NC)cc2)nc(O[C@@H]2C([2H])COC[C@@H]2O)c1C(F)(F)F]

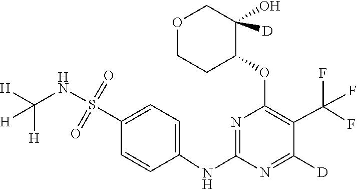 [SMILES: [2H]c1nc(Nc2ccc(S(=O)(=O)NC)cc2)nc(O[C@@H]2CCOC[C@]2([2H])O)c1C(F)(F)F]

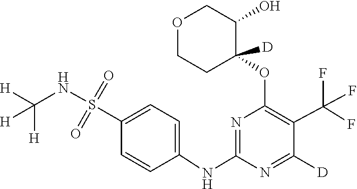 [SMILES: [2H]c1nc(Nc2ccc(S(=O)(=O)NC)cc2)nc(O[C@]2([2H])CCOC[C@@H]2O)c1C(F)(F)F]

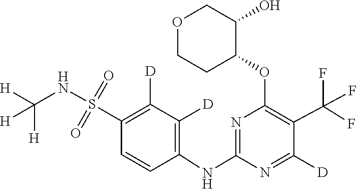 [SMILES: [2H]c1nc(Nc2ccc(S(=O)(=O)NC)c([2H])c2[2H])nc(O[C@@H]2CCOC[C@@H]2O)c1C(F)(F)F]

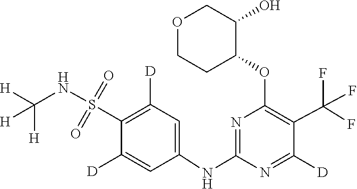 [SMILES: [2H]c1cc(Nc2nc([2H])c(C(F)(F)F)c(O[C@@H]3CCOC[C@@H]3O)n2)cc([2H])c1S(=O)(=O)NC]

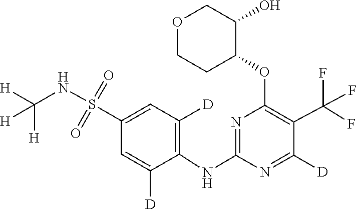 [SMILES: [2H]c1cc(S(=O)(=O)NC)cc([2H])c1Nc1nc([2H])c(C(F)(F)F)c(O[C@@H]2CCOC[C@@H]2O)n1]

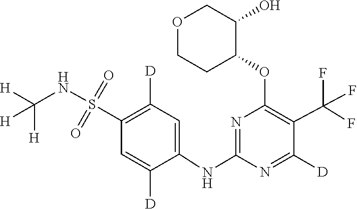 [SMILES: [2H]c1cc(S(=O)(=O)NC)c([2H])cc1Nc1nc([2H])c(C(F)(F)F)c(O[C@@H]2CCOC[C@@H]2O)n1]

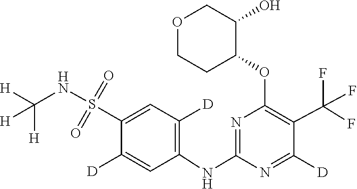 [SMILES: [2H]c1cc(S(=O)(=O)NC)c([2H])cc1Nc1nc([2H])c(C(F)(F)F)c(O[C@@H]2CCOC[C@@H]2O)n1]

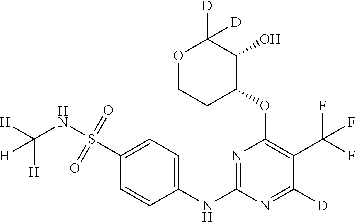 [SMILES: [2H]c1nc(Nc2ccc(S(=O)(=O)NC)cc2)nc(O[C@@H]2CCOC([2H])([2H])[C@@H]2O)c1C(F)(F)F]

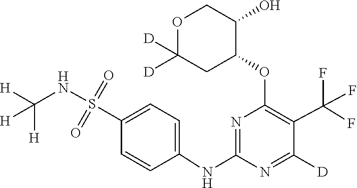 [SMILES: [2H]c1nc(Nc2ccc(S(=O)(=O)NC)cc2)nc(O[C@@H]2CC([2H])([2H])OC[C@@H]2O)c1C(F)(F)F]

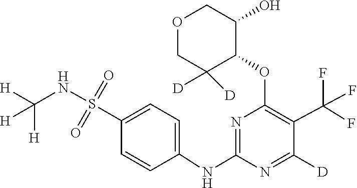 [SMILES: [2H]c1nc(Nc2ccc(S(=O)(=O)NC)cc2)nc(O[C@H]2[C@@H](O)COCC2([2H])[2H])c1C(F)(F)F]

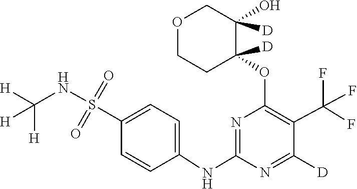 [SMILES: [2H]c1nc(Nc2ccc(S(=O)(=O)NC)cc2)nc(O[C@]2([2H])CCOC[C@]2([2H])O)c1C(F)(F)F]

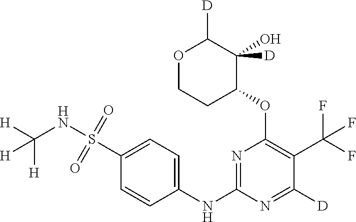 [SMILES: [2H]c1nc(Nc2ccc(S(=O)(=O)NC)cc2)nc(O[C@@H]2CCOC([2H])[C@]2([2H])O)c1C(F)(F)F]

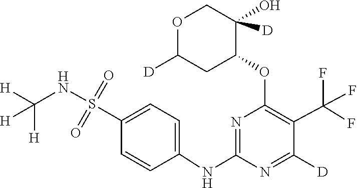 [SMILES: [2H]c1nc(Nc2ccc(S(=O)(=O)NC)cc2)nc(O[C@@H]2CC([2H])OC[C@]2([2H])O)c1C(F)(F)F]

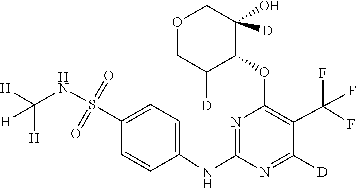 [SMILES: [2H]c1nc(Nc2ccc(S(=O)(=O)NC)cc2)nc(O[C@@H]2C([2H])COC[C@]2([2H])O)c1C(F)(F)F]

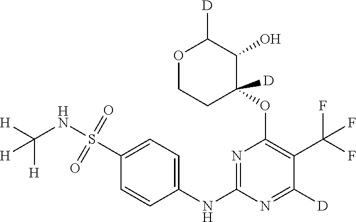 [SMILES: [2H]c1nc(Nc2ccc(S(=O)(=O)NC)cc2)nc(O[C@]2([2H])CCOC([2H])[C@@H]2O)c1C(F)(F)F]

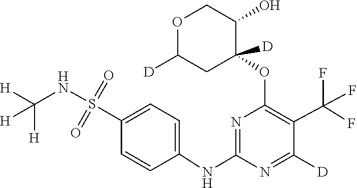 [SMILES: [2H]c1nc(Nc2ccc(S(=O)(=O)NC)cc2)nc(O[C@]2([2H])CC([2H])OC[C@@H]2O)c1C(F)(F)F]

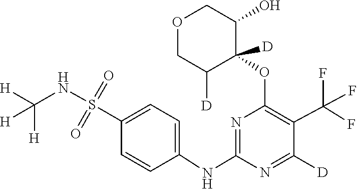 [SMILES: [2H]c1nc(Nc2ccc(S(=O)(=O)NC)cc2)nc(O[C@]2([2H])C([2H])COC[C@@H]2O)c1C(F)(F)F]

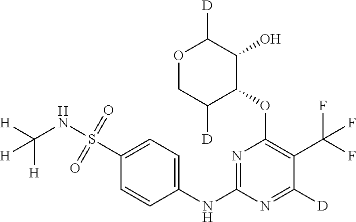 [SMILES: [2H]c1nc(Nc2ccc(S(=O)(=O)NC)cc2)nc(O[C@@H]2C([2H])COC([2H])[C@@H]2O)c1C(F)(F)F]

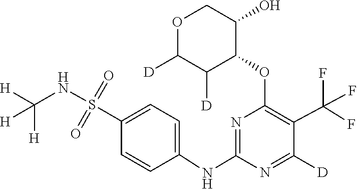 [SMILES: [2H]c1nc(Nc2ccc(S(=O)(=O)NC)cc2)nc(O[C@@H]2C([2H])C([2H])OC[C@@H]2O)c1C(F)(F)F]

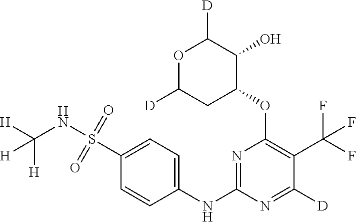 [SMILES: [2H]c1nc(Nc2ccc(S(=O)(=O)NC)cc2)nc(O[C@@H]2CC([2H])OC([2H])[C@@H]2O)c1C(F)(F)F]

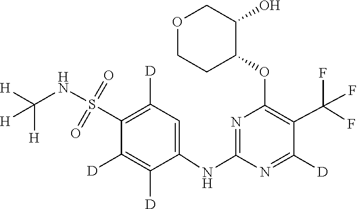 [SMILES: [2H]c1cc(Nc2nc([2H])c(C(F)(F)F)c(O[C@@H]3CCOC[C@@H]3O)n2)c([2H])c([2H])c1S(=O)(=O)NC]

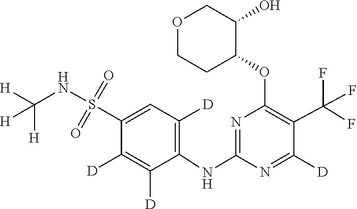 [SMILES: [2H]c1cc(S(=O)(=O)NC)c([2H])c([2H])c1Nc1nc([2H])c(C(F)(F)F)c(O[C@@H]2CCOC[C@@H]2O)n1]

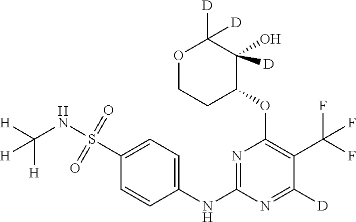 [SMILES: [2H]c1nc(Nc2ccc(S(=O)(=O)NC)cc2)nc(O[C@@H]2CCOC([2H])([2H])[C@]2([2H])O)c1C(F)(F)F]

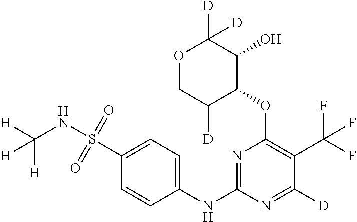 [SMILES: [2H]c1nc(Nc2ccc(S(=O)(=O)NC)cc2)nc(O[C@@H]2C([2H])COC([2H])([2H])[C@@H]2O)c1C(F)(F)F]

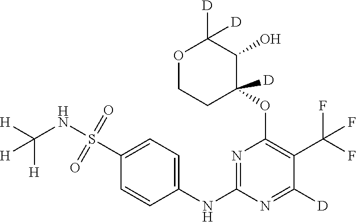 [SMILES: [2H]c1nc(Nc2ccc(S(=O)(=O)NC)cc2)nc(O[C@]2([2H])CCOC([2H])([2H])[C@@H]2O)c1C(F)(F)F]

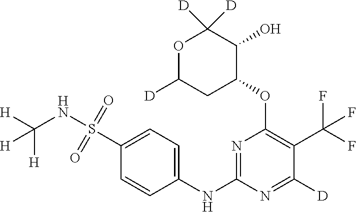 [SMILES: [2H]c1nc(Nc2ccc(S(=O)(=O)NC)cc2)nc(O[C@@H]2CC([2H])OC([2H])([2H])[C@@H]2O)c1C(F)(F)F]

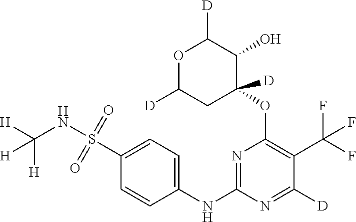 [SMILES: [2H]c1nc(Nc2ccc(S(=O)(=O)NC)cc2)nc(O[C@]2([2H])CC([2H])OC([2H])[C@@H]2O)c1C(F)(F)F]

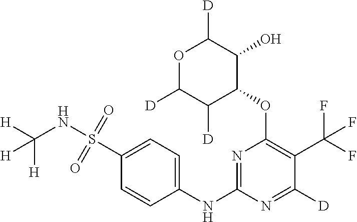 [SMILES: [2H]c1nc(Nc2ccc(S(=O)(=O)NC)cc2)nc(O[C@@H]2C([2H])C([2H])OC([2H])[C@@H]2O)c1C(F)(F)F]

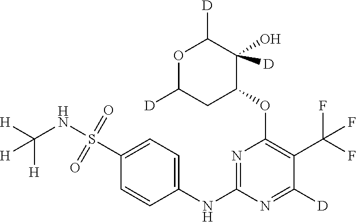 [SMILES: [2H]c1nc(Nc2ccc(S(=O)(=O)NC)cc2)nc(O[C@@H]2CC([2H])OC([2H])[C@]2([2H])O)c1C(F)(F)F]

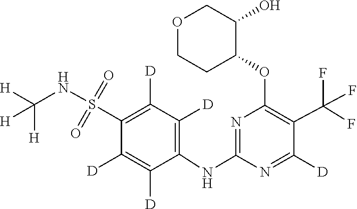 [SMILES: [2H]c1nc(Nc2c([2H])c([2H])c(S(=O)(=O)NC)c([2H])c2[2H])nc(O[C@@H]2CCOC[C@@H]2O)c1C(F)(F)F]

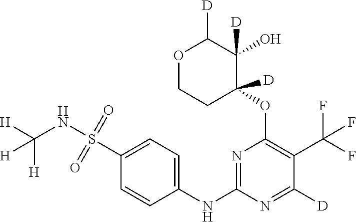 [SMILES: [2H]c1nc(Nc2ccc(S(=O)(=O)NC)cc2)nc(O[C@]2([2H])CCOC([2H])[C@]2([2H])O)c1C(F)(F)F]

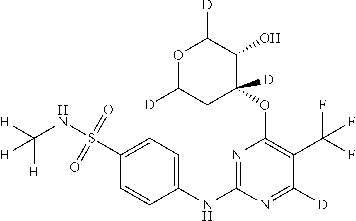 [SMILES: [2H]c1nc(Nc2ccc(S(=O)(=O)NC)cc2)nc(O[C@]2([2H])CC([2H])OC([2H])[C@@H]2O)c1C(F)(F)F]

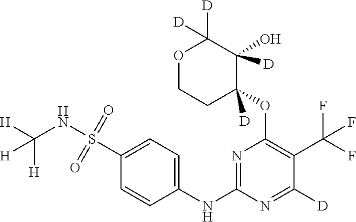 [SMILES: [2H]c1nc(Nc2ccc(S(=O)(=O)NC)cc2)nc(O[C@]2([2H])CCOC([2H])([2H])[C@]2([2H])O)c1C(F)(F)F]

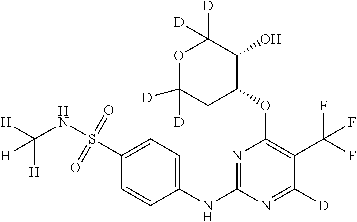 [SMILES: [2H]c1nc(Nc2ccc(S(=O)(=O)NC)cc2)nc(O[C@@H]2CC([2H])([2H])OC([2H])([2H])[C@@H]2O)c1C(F)(F)F]

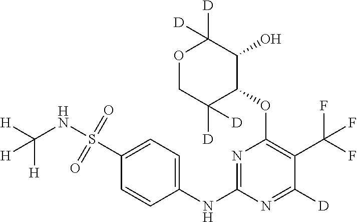 [SMILES: [2H]c1nc(Nc2ccc(S(=O)(=O)NC)cc2)nc(O[C@H]2[C@@H](O)C([2H])([2H])OCC2([2H])[2H])c1C(F)(F)F]

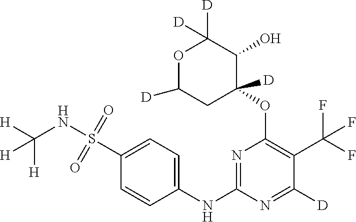 [SMILES: [2H]c1nc(Nc2ccc(S(=O)(=O)NC)cc2)nc(O[C@]2([2H])CC([2H])OC([2H])([2H])[C@@H]2O)c1C(F)(F)F]

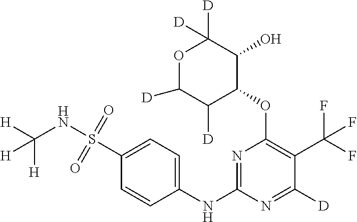 [SMILES: [2H]c1nc(Nc2ccc(S(=O)(=O)NC)cc2)nc(O[C@@H]2C([2H])C([2H])OC([2H])([2H])[C@@H]2O)c1C(F)(F)F]

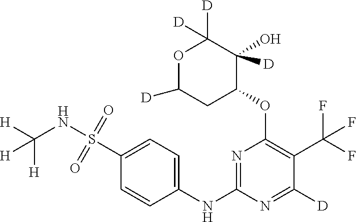 [SMILES: [2H]c1nc(Nc2ccc(S(=O)(=O)NC)cc2)nc(O[C@@H]2CC([2H])OC([2H])([2H])[C@]2([2H])O)c1C(F)(F)F]

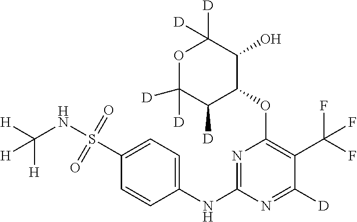 [SMILES: [2H]c1nc(Nc2ccc(S(=O)(=O)NC)cc2)nc(O[C@@H]2[C@@H]([2H])C([2H])([2H])OC([2H])([2H])[C@@H]2O)c1C(F)(F)F]

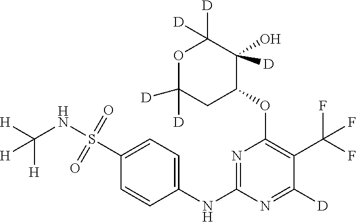 [SMILES: [2H]c1nc(Nc2ccc(S(=O)(=O)NC)cc2)nc(O[C@@H]2CC([2H])([2H])OC([2H])([2H])[C@]2([2H])O)c1C(F)(F)F]

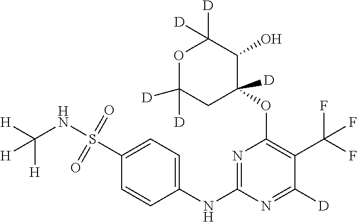 [SMILES: [2H]c1nc(Nc2ccc(S(=O)(=O)NC)cc2)nc(O[C@]2([2H])CC([2H])([2H])OC([2H])([2H])[C@@H]2O)c1C(F)(F)F]

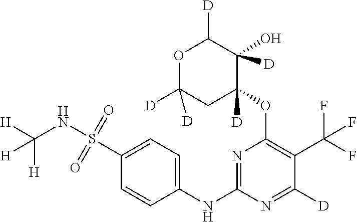 [SMILES: [2H]c1nc(Nc2ccc(S(=O)(=O)NC)cc2)nc(O[C@]2([2H])CC([2H])([2H])OC([2H])[C@]2([2H])O)c1C(F)(F)F]

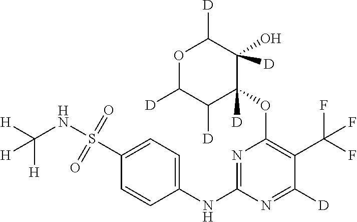 [SMILES: [2H]c1nc(Nc2ccc(S(=O)(=O)NC)cc2)nc(O[C@]2([2H])C([2H])C([2H])OC([2H])[C@]2([2H])O)c1C(F)(F)F]

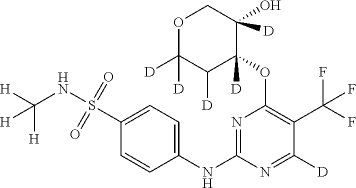 [SMILES: [2H]c1nc(Nc2ccc(S(=O)(=O)NC)cc2)nc(O[C@]2([2H])C([2H])C([2H])([2H])OC[C@]2([2H])O)c1C(F)(F)F]

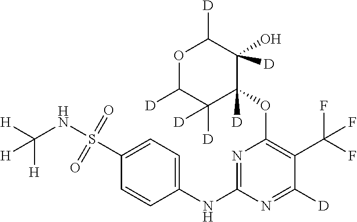 [SMILES: [2H]c1nc(Nc2ccc(S(=O)(=O)NC)cc2)nc(O[C@]2([2H])C([2H])([2H])C([2H])OC([2H])[C@]2([2H])O)c1C(F)(F)F]

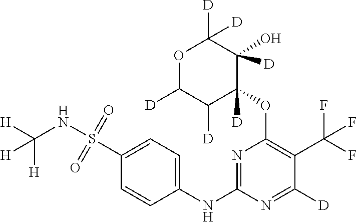 [SMILES: [2H]c1nc(Nc2ccc(S(=O)(=O)NC)cc2)nc(O[C@]2([2H])C([2H])C([2H])OC([2H])([2H])[C@]2([2H])O)c1C(F)(F)F]

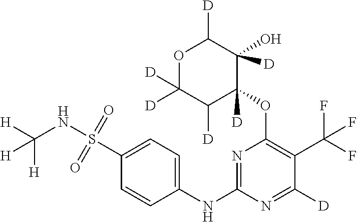 [SMILES: [2H]c1nc(Nc2ccc(S(=O)(=O)NC)cc2)nc(O[C@]2([2H])C([2H])C([2H])([2H])OC([2H])[C@]2([2H])O)c1C(F)(F)F]

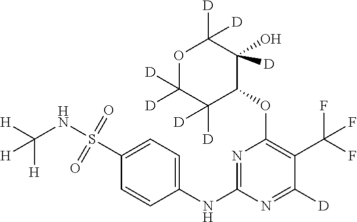 [SMILES: [2H]c1nc(Nc2ccc(S(=O)(=O)NC)cc2)nc(O[C@@H]2C([2H])([2H])C([2H])([2H])OC([2H])([2H])[C@]2([2H])O)c1C(F)(F)F]

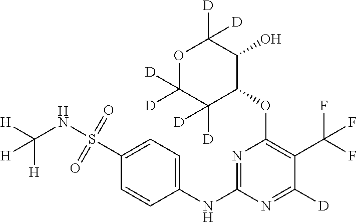 [SMILES: [2H]c1nc(Nc2ccc(S(=O)(=O)NC)cc2)nc(O[C@H]2[C@@H](O)C([2H])([2H])OC([2H])([2H])C2([2H])[2H])c1C(F)(F)F]

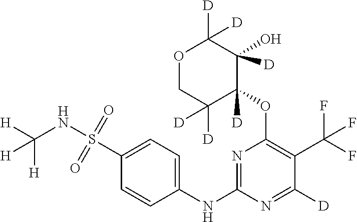 [SMILES: [2H]c1nc(Nc2ccc(S(=O)(=O)NC)cc2)nc(O[C@]2([2H])C([2H])([2H])COC([2H])([2H])[C@]2([2H])O)c1C(F)(F)F]

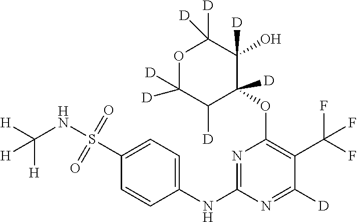 [SMILES: [2H]c1nc(Nc2ccc(S(=O)(=O)NC)cc2)nc(O[C@]2([2H])C([2H])C([2H])([2H])OC([2H])([2H])[C@]2([2H])O)c1C(F)(F)F]

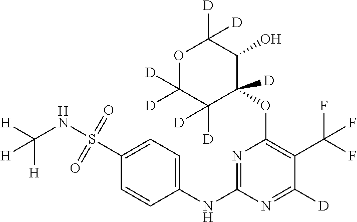 [SMILES: [2H]c1nc(Nc2ccc(S(=O)(=O)NC)cc2)nc(O[C@@]2([2H])[C@@H](O)C([2H])([2H])OC([2H])([2H])C2([2H])[2H])c1C(F)(F)F]

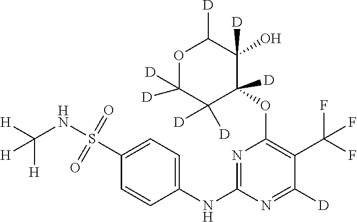 [SMILES: [2H]c1nc(Nc2ccc(S(=O)(=O)NC)cc2)nc(O[C@]2([2H])C([2H])([2H])C([2H])([2H])OC([2H])[C@]2([2H])O)c1C(F)(F)F]

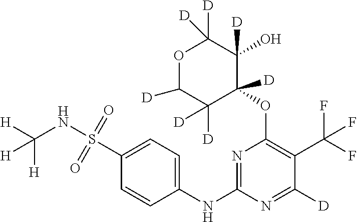 [SMILES: [2H]c1nc(Nc2ccc(S(=O)(=O)NC)cc2)nc(O[C@]2([2H])C([2H])([2H])C([2H])OC([2H])([2H])[C@]2([2H])O)c1C(F)(F)F]

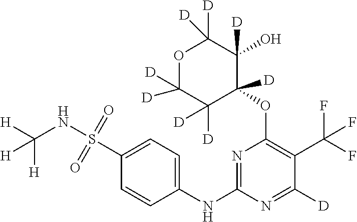 [SMILES: [2H]c1nc(Nc2ccc(S(=O)(=O)NC)cc2)nc(O[C@]2([2H])C([2H])([2H])C([2H])([2H])OC([2H])([2H])[C@]2([2H])O)c1C(F)(F)F]

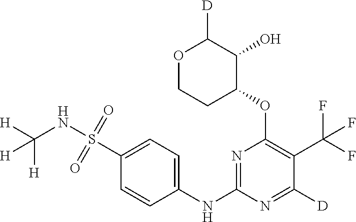 [SMILES: [2H]c1nc(Nc2ccc(S(=O)(=O)NC)cc2)nc(O[C@@H]2CCOC([2H])[C@@H]2O)c1C(F)(F)F]

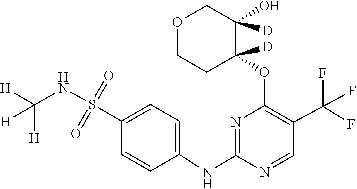 [SMILES: [2H][C@]1(O)COCC[C@@]1([2H])Oc1nc(Nc2ccc(S(=O)(=O)NC)cc2)ncc1C(F)(F)F]

In some embodiments, the compound is a compound of Formula (III-A), (III-B), (III-Q), (III-D), (III-E), (Ill-F), (III-G), or (Ill-H) selected from any one of the compounds in Table 5 or 6.

TABLE 5
Exemplary compounds of Formula (III-A), (III-B), (III-
C), (III-D), (III-E), (III-F), (III-G), or (III-H).

| Row | Y⁵ | Y⁶ | Y⁷ | Y⁸ | Y⁹ | Y¹⁰ | Y¹¹ | Y¹² | Y¹³ | Y¹⁴ | Y¹⁵ | Y¹⁶ |
|---|---|---|---|---|---|---|---|---|---|---|---|---|
| 1. | H | H | H | H | H | H | H | H | H | H | D | D |
| 2. | D | H | H | H | H | H | H | H | H | H | D | D |
| 3. | H | D | H | H | H | H | H | H | H | H | D | D |
| 4. | D | D | H | H | H | H | H | H | H | H | D | D |
| 5. | D | H | D | H | H | H | H | H | H | H | D | D |
| 6. | D | H | H | D | H | H | H | H | H | H | D | D |
| 7. | H | D | D | H | H | H | H | H | H | H | D | D |
| 8. | H | H | D | D | H | H | H | H | H | H | D | D |
| 9. | D | D | D | H | H | H | H | H | H | H | D | D |
| 10. | D | D | H | D | H | H | H | H | H | H | D | D |
| 11. | H | D | D | D | H | H | H | H | H | H | D | D |
| 12. | D | H | D | D | H | H | H | H | H | H | D | D |
| 13. | D | D | D | D | H | H | H | H | H | H | D | D |
| 14. | H | H | H | H | H | H | H | H | H | H | D | H |
| 15. | D | H | H | H | H | H | H | H | H | H | D | H |
| 16. | H | D | H | H | H | H | H | H | H | H | D | H |
| 17. | D | D | H | H | H | H | H | H | H | H | D | H |
| 18. | D | H | D | H | H | H | H | H | H | H | D | H |
| 19. | D | H | H | D | H | H | H | H | H | H | D | H |
| 20. | H | D | D | H | H | H | H | H | H | H | D | H |
| 21. | H | H | D | D | H | H | H | H | H | H | D | H |
| 22. | D | D | D | H | H | H | H | H | H | H | D | H |
| 23. | D | D | H | D | H | H | H | H | H | H | D | H |
| 24. | H | D | D | D | H | H | H | H | H | H | D | H |
| 25. | D | H | D | D | H | H | H | H | H | H | D | H |
| 26. | D | D | D | D | H | H | H | H | H | H | D | H |
| 27. | H | H | H | H | H | H | H | H | H | H | H | D |
| 28. | D | H | H | H | H | H | H | H | H | H | H | D |
| 29. | H | D | H | H | H | H | H | H | H | H | H | D |
| 30. | D | D | H | H | H | H | H | H | H | H | H | D |
| 31. | D | H | D | H | H | H | H | H | H | H | H | D |
| 32. | D | H | H | D | H | H | H | H | H | H | H | D |
| 33. | H | D | D | H | H | H | H | H | H | H | H | D |
| 34. | H | H | D | D | H | H | H | H | H | H | H | D |
| 35. | D | D | D | H | H | H | H | H | H | H | H | D |
| 36. | D | D | H | D | H | H | H | H | H | H | H | D |
| 37. | H | D | D | D | H | H | H | H | H | H | H | D |
| 38. | D | H | D | D | H | H | H | H | H | H | H | D |
| 39. | D | D | D | D | H | H | H | H | H | H | H | D |
| 40. | H | H | H | H | D | D | D | H | H | H | H | H |
| 41. | D | H | H | H | D | D | D | H | H | H | H | H |
| 42. | H | D | H | H | D | D | D | H | H | H | H | H |
| 43. | D | D | H | H | D | D | D | H | H | H | H | H |
| 44. | D | H | D | H | D | D | D | H | H | H | H | H |
| 45. | D | H | H | D | D | D | D | H | H | H | H | H |
| 46. | H | D | D | H | D | D | D | H | H | H | H | H |
| 47. | H | H | D | D | D | D | D | H | H | H | H | H |
| 48. | D | D | D | H | D | D | D | H | H | H | H | H |
| 49. | D | D | H | D | D | D | D | H | H | H | H | H |
| 50. | H | D | D | D | D | D | D | H | H | H | H | H |
| 51. | D | H | D | D | D | D | D | H | H | H | H | H |
| 52. | D | D | D | D | D | D | D | H | H | H | H | H |
| 53. | H | H | H | H | D | D | H | H | D | H | H | H |
| 54. | D | H | H | H | D | D | H | H | D | H | H | H |
| 55. | H | D | H | H | D | D | H | H | D | H | H | H |
| 56. | D | D | H | H | D | D | H | H | D | H | H | H |
| 57. | D | H | D | H | D | D | H | H | D | H | H | H |
| 58. | D | H | H | D | D | D | H | H | D | H | H | H |
| 59. | H | D | D | H | D | D | H | H | D | H | H | H |
| 60. | H | H | D | D | D | D | H | H | D | H | H | H |
| 61. | D | D | D | H | D | D | H | H | D | H | H | H |
| 62. | D | D | H | D | D | D | H | H | D | H | H | H |
| 63. | H | D | D | D | D | D | H | H | D | H | H | H |
| 64. | D | H | D | D | D | D | H | H | D | H | H | H |
| 65. | D | D | D | D | D | D | H | H | D | H | H | H |
| 66. | H | H | H | H | D | D | H | H | H | H | D | H |
| 67. | D | H | H | H | D | D | H | H | H | H | D | H |
| 68. | H | D | H | H | D | D | H | H | H | H | D | H |
| 69. | D | D | H | H | D | D | H | H | H | H | D | H |
| 70. | D | H | D | H | D | D | H | H | H | H | D | H |
| 71. | D | H | H | D | D | D | H | H | H | H | D | H |
| 72. | H | D | D | H | D | D | H | H | H | H | D | H |
| 73. | H | H | D | D | D | D | H | H | H | H | D | H |
| 74. | D | D | D | H | D | D | H | H | H | H | D | H |
| 75. | D | D | H | D | D | D | H | H | H | H | D | H |
| 76. | H | D | D | D | D | D | H | H | H | H | D | H |
| 77. | D | H | D | D | D | D | H | H | H | H | D | H |
| 78. | D | D | D | D | D | D | H | H | H | H | D | H |
| 79. | H | H | H | H | D | D | H | H | H | H | H | D |
| 80. | D | H | H | H | D | D | H | H | H | H | H | D |
| 81. | H | D | H | H | D | D | H | H | H | H | H | D |
| 82. | D | D | H | H | D | D | H | H | H | H | H | D |
| 83. | D | H | D | H | D | D | H | H | H | H | H | D |
| 84. | D | H | H | D | D | D | H | H | H | H | H | D |
| 85. | H | D | D | H | D | D | H | H | H | H | H | D |
| 86. | H | H | D | D | D | D | H | H | H | H | H | D |
| 87. | D | D | D | H | D | D | H | H | H | H | H | D |
| 88. | D | D | H | D | D | D | H | H | H | H | H | D |
| 89. | H | D | D | D | D | D | H | H | H | H | H | D |
| 90. | D | H | D | D | D | D | H | H | H | H | H | D |
| 91. | D | D | D | D | D | D | H | H | H | H | H | D |
| 92. | H | H | H | H | D | D | D | D | H | H | H | H |
| 93. | D | H | H | H | D | D | D | D | H | H | H | H |
| 94. | H | D | H | H | D | D | D | D | H | H | H | H |
| 95. | D | D | H | H | D | D | D | D | H | H | H | H |
| 96. | D | H | D | H | D | D | D | D | H | H | H | H |
| 97. | D | H | H | D | D | D | D | D | H | H | H | H |
| 98. | H | D | D | H | D | D | D | D | H | H | H | H |
| 99. | H | H | D | D | D | D | D | D | H | H | H | H |
| 100. | D | D | D | H | D | D | D | D | H | H | H | H |
| 101. | D | D | H | D | D | D | D | D | H | H | H | H |
| 102. | H | D | D | D | D | D | D | D | H | H | H | H |
| 103. | D | H | D | D | D | D | D | D | H | H | H | H |
| 104. | D | D | D | D | D | D | D | D | H | H | H | H |
| 105. | H | H | H | H | D | D | H | H | D | D | H | H |
| 106. | D | H | H | H | D | D | H | H | D | D | H | H |
| 107. | H | D | H | H | D | D | H | H | D | D | H | H |
| 108. | D | D | H | H | D | D | H | H | D | D | H | H |
| 109. | D | H | D | H | D | D | H | H | D | D | H | H |
| 110. | D | H | H | D | D | D | H | H | D | D | H | H |
| 111. | H | D | D | H | D | D | H | H | D | D | H | H |
| 112. | H | H | D | D | D | D | H | H | D | D | H | H |
| 113. | D | D | D | H | D | D | H | H | D | D | H | H |
| 114. | D | D | H | D | D | D | H | H | D | D | H | H |
| 115. | H | D | D | D | D | D | H | H | D | D | H | H |
| 116. | D | H | D | D | D | D | H | H | D | D | H | H |
| 117. | D | D | D | D | D | D | H | H | D | D | H | H |
| 118. | H | H | H | H | D | D | D | H | D | H | H | H |
| 119. | D | H | H | H | D | D | D | H | D | H | H | H |
| 120. | H | D | H | H | D | D | D | H | D | H | H | H |
| 121. | D | D | H | H | D | D | D | H | D | H | H | H |
| 122. | D | H | D | H | D | D | D | H | D | H | H | H |
| 123. | D | H | H | D | D | D | D | H | D | H | H | H |
| 124. | H | D | D | H | D | D | D | H | D | H | H | H |
| 125. | H | H | D | D | D | D | D | H | D | H | H | H |
| 126. | D | D | D | H | D | D | D | H | D | H | H | H |
| 127. | D | D | H | D | D | D | D | H | D | H | H | H |
| 128. | H | D | D | D | D | D | D | H | D | H | H | H |
| 129. | D | H | D | D | D | D | D | H | D | H | H | H |
| 130. | D | D | D | D | D | D | D | H | D | H | H | H |
| 131. | C | H | H | H | D | D | D | H | H | H | D | H |
| 132. | D | H | H | H | D | D | D | H | H | H | D | H |
| 133. | H | D | H | H | D | D | D | H | H | H | D | H |
| 134. | D | D | H | H | D | D | D | H | H | H | D | H |
| 135. | D | H | D | H | D | D | D | H | H | H | D | H |
| 136. | D | H | H | D | D | D | D | H | H | H | D | H |
| 137. | H | D | D | H | D | D | D | H | H | H | D | H |
| 138. | H | H | D | D | D | D | D | H | H | H | D | H |
| 139. | D | D | D | H | D | D | D | H | H | H | D | H |
| 140. | D | D | H | D | D | D | D | H | H | H | D | H |
| 141. | H | D | D | D | D | D | D | H | H | H | D | H |
| 142. | D | H | D | D | D | D | D | H | H | H | D | H |
| 143. | D | D | D | D | D | D | D | H | H | H | D | H |
| 144. | H | H | H | H | D | D | D | H | H | H | H | D |
| 145. | D | H | H | H | D | D | D | H | H | H | H | D |
| 146. | H | D | H | H | D | D | D | H | H | H | H | D |
| 147. | D | D | H | H | D | D | D | H | H | H | H | D |
| 148. | D | H | D | H | D | D | D | H | H | H | H | D |
| 149. | D | H | H | D | D | D | D | H | H | H | H | D |
| 150. | H | D | D | H | D | D | D | H | H | H | H | D |
| 151. | H | H | D | D | D | D | D | H | H | H | H | D |
| 152. | D | D | D | H | D | D | D | H | H | H | H | D |
| 153. | D | D | H | D | D | D | D | H | H | H | H | D |
| 154. | H | D | D | D | D | D | D | H | H | H | H | D |
| 155. | D | H | D | D | D | D | D | H | H | H | H | D |
| 156. | D | D | D | D | D | D | D | H | H | H | H | D |
| 157. | H | H | H | H | D | D | H | H | H | H | D | D |
| 158. | D | H | H | H | D | D | H | H | H | H | D | D |
| 159. | H | D | H | H | D | D | H | H | H | H | D | D |
| 160. | D | D | H | H | D | D | H | H | H | H | D | D |
| 161. | D | H | D | H | D | D | H | H | H | H | D | D |
| 162. | D | H | H | D | D | D | H | H | H | H | D | D |
| 163. | H | D | D | H | D | D | H | H | H | H | D | D |
| 164. | H | H | D | D | D | D | H | H | H | H | D | D |
| 165. | D | D | D | H | D | D | H | H | H | H | D | D |
| 166. | D | D | H | D | D | D | H | H | H | H | D | D |
| 167. | H | D | D | D | D | D | H | H | H | H | D | D |
| 168. | D | H | D | D | D | D | H | H | H | H | D | D |
| 169. | D | D | D | D | D | D | H | H | H | H | D | D |
| 170. | H | H | H | H | D | H | D | D | D | H | H | H |
| 171. | D | H | H | H | D | H | D | D | D | H | H | H |
| 172. | H | D | H | H | D | H | D | D | D | H | H | H |
| 173. | D | D | H | H | D | H | D | D | D | H | H | H |
| 174. | D | H | D | H | D | H | D | D | D | H | H | H |
| 175. | D | H | H | D | D | H | D | D | D | H | H | H |
| 176. | H | D | D | H | D | H | D | D | D | H | H | H |
| 177. | H | H | D | D | D | H | D | D | D | H | H | H |
| 178. | D | D | D | H | D | H | D | D | D | H | H | H |
| 179. | D | D | H | D | D | H | D | D | D | H | H | H |
| 180. | H | D | D | D | D | H | D | D | D | H | H | H |
| 181. | D | H | D | D | D | H | D | D | D | H | H | H |
| 182. | D | D | D | D | D | H | D | D | D | H | H | H |
| 183. | H | H | H | H | D | H | D | D | H | H | D | H |
| 184. | D | H | H | H | D | H | D | D | H | H | D | H |
| 185. | H | D | H | H | D | H | D | D | H | H | D | H |
| 186. | D | D | H | H | D | H | D | D | H | H | D | H |
| 187. | D | H | D | H | D | H | D | D | H | H | D | H |
| 188. | D | H | H | D | D | H | D | D | H | H | D | H |
| 189. | H | D | D | H | D | H | D | D | H | H | D | H |
| 190. | H | H | D | D | D | H | D | D | H | H | D | H |
| 191. | D | D | D | H | D | H | D | D | H | H | D | H |
| 192. | D | D | H | D | D | H | D | D | H | H | D | H |
| 193. | H | D | D | D | D | H | D | D | H | H | D | H |
| 194. | D | H | D | D | D | H | D | D | H | H | D | H |
| 195. | D | D | D | D | D | H | D | D | H | H | D | H |
| 196. | H | H | H | H | D | H | D | D | H | H | H | D |
| 197. | D | H | H | H | D | H | D | D | H | H | H | D |
| 198. | H | D | H | H | D | H | D | D | H | H | H | D |
| 199. | D | D | H | H | D | H | D | D | H | H | H | D |
| 200. | D | H | D | H | D | H | D | D | H | H | H | D |
| 201. | D | H | H | D | D | H | D | D | H | H | H | D |
| 202. | H | D | D | H | D | H | D | D | H | H | H | D |
| 203. | H | H | D | D | D | H | D | D | H | H | H | D |
| 204. | D | D | D | H | D | H | D | D | H | H | H | D |
| 205. | D | D | H | D | D | H | D | D | H | H | H | D |
| 206. | H | D | D | D | D | H | D | D | H | H | H | D |
| 207. | D | H | D | D | D | H | D | D | H | H | H | D |
| 208. | D | D | D | D | D | H | D | D | H | H | H | D |
| 209. | H | H | H | H | D | H | D | H | D | D | H | H |
| 210. | D | H | H | H | D | H | D | H | D | D | H | H |
| 211. | H | D | H | H | D | H | D | H | D | D | H | H |
| 212. | D | D | H | H | D | H | D | H | D | D | H | H |
| 213. | D | H | D | H | D | H | D | H | D | D | H | H |
| 214. | D | H | H | D | D | H | D | H | D | D | H | H |
| 215. | H | D | D | H | D | H | D | H | D | D | H | H |
| 216. | H | H | D | D | D | H | D | H | D | D | H | H |
| 217. | D | D | D | H | D | H | D | H | D | D | H | H |
| 218. | D | D | H | D | D | H | D | H | D | D | H | H |
| 219. | H | D | D | D | D | H | D | H | D | D | H | H |
| 220. | D | H | D | D | D | H | D | H | D | D | H | H |
| 221. | D | D | D | D | D | H | D | H | D | D | H | H |
| 222. | H | H | H | H | D | H | D | H | D | H | D | H |
| 223. | D | H | H | H | D | H | D | H | D | H | D | H |
| 224. | H | D | H | H | D | H | D | H | D | H | D | H |
| 225. | D | D | H | H | D | H | D | H | D | H | D | H |
| 226. | D | H | D | H | D | H | D | H | D | H | D | H |
| 227. | D | H | H | D | D | H | D | H | D | H | D | H |
| 228. | H | D | D | H | D | H | D | H | D | H | D | H |
| 229. | H | H | D | D | D | H | D | H | D | H | D | H |
| 230. | D | D | D | H | D | H | D | H | D | H | D | H |
| 231. | D | D | H | D | D | H | D | H | D | H | D | H |
| 232. | H | D | D | D | D | H | D | H | D | H | D | H |
| 233. | D | H | D | D | D | H | D | H | D | H | D | H |
| 234. | D | D | D | D | D | H | D | H | D | H | D | H |
| 235. | H | H | H | H | D | H | D | H | D | H | H | D |
| 236. | D | H | H | H | D | H | D | H | D | H | H | D |
| 237. | H | D | H | H | D | H | D | H | D | H | H | D |
| 238. | D | D | H | H | D | H | D | H | D | H | H | D |
| 239. | D | H | D | H | D | H | D | H | D | H | H | D |
| 240. | D | H | H | D | D | H | D | H | D | H | H | D |
| 241. | H | D | D | H | D | H | D | H | D | H | H | D |
| 242. | H | H | D | D | D | H | D | H | D | H | H | D |
| 243. | D | D | D | H | D | H | D | H | D | H | H | D |
| 244. | D | D | H | D | D | H | D | H | D | H | H | D |
| 245. | H | D | D | D | D | H | D | H | D | H | H | D |
| 246. | D | H | D | D | D | H | D | H | D | H | H | D |
| 247. | D | D | D | D | D | H | D | H | D | H | H | D |
| 248. | H | H | H | H | H | H | D | D | D | D | H | H |
| 249. | D | H | H | H | H | H | D | D | D | D | H | H |
| 250. | H | D | H | H | H | H | D | D | D | D | H | H |
| 251. | D | D | H | H | H | H | D | D | D | D | H | H |
| 252. | D | H | D | H | H | H | D | D | D | D | H | H |
| 253. | D | H | H | D | H | H | D | D | D | D | H | H |
| 254. | H | D | D | H | H | H | D | D | D | D | H | H |
| 255. | H | H | D | D | H | H | D | D | D | D | H | H |
| 256. | D | D | D | H | H | H | D | D | D | D | H | H |
| 257. | D | D | H | D | H | H | D | D | D | D | H | H |
| 258. | D | D | D | D | H | H | D | D | D | D | H | H |
| 259. | H | H | H | H | H | H | D | D | H | D | D | H |
| 260. | D | H | H | H | H | H | D | D | H | D | D | H |
| 261. | H | D | H | H | H | H | D | D | H | D | D | H |
| 262. | D | D | H | H | H | H | D | D | H | D | D | H |
| 263. | D | H | D | H | H | H | D | D | H | D | D | H |
| 264. | D | H | H | D | H | H | D | D | H | D | D | H |
| 265. | H | D | D | H | H | H | D | D | H | D | D | H |
| 266. | H | H | D | D | H | H | D | D | H | D | D | H |
| 267. | D | D | D | H | H | H | D | D | H | D | D | H |
| 268. | D | D | H | D | H | H | D | D | H | D | D | H |
| 269. | D | D | D | D | H | H | D | D | H | D | D | H |
| 270. | H | H | H | H | H | H | D | D | H | D | H | D |
| 271. | D | H | H | H | H | H | D | D | H | D | H | D |
| 272. | H | D | H | H | H | H | D | D | H | D | H | D |
| 273. | D | D | H | H | H | H | D | D | H | D | H | D |
| 274. | D | H | D | H | H | H | D | D | H | D | H | D |
| 275. | D | H | H | D | H | H | D | D | H | D | H | D |
| 276. | H | D | D | H | H | H | D | D | H | D | H | D |
| 277. | H | H | D | D | H | H | D | D | H | D | H | D |
| 278. | D | D | D | H | H | H | D | D | H | D | H | D |
| 279. | D | D | H | D | H | H | D | D | H | D | H | D |
| 280. | D | D | D | D | H | H | D | D | H | D | H | D |
| 281. | H | H | H | H | H | H | D | D | H | H | D | D |
| 282. | D | H | H | H | H | H | D | D | H | H | D | D |
| 283. | H | D | H | H | H | H | D | D | H | H | D | D |
| 284. | D | D | H | H | H | H | D | D | H | H | D | D |
| 285. | D | H | D | H | H | H | D | D | H | H | D | D |
| 286. | D | H | H | D | H | H | D | D | H | H | D | D |
| 287. | H | D | D | H | H | H | D | D | H | H | D | D |
| 288. | H | H | D | D | H | H | D | D | H | H | D | D |
| 289. | D | D | D | H | H | H | D | D | H | H | D | D |
| 290. | D | D | H | D | H | H | D | D | H | H | D | D |
| 291. | D | D | D | D | H | H | D | D | H | H | D | D |
| 292. | H | H | H | H | H | H | D | H | D | D | D | H |
| 293. | D | H | H | H | H | H | D | H | D | D | D | H |
| 294. | H | D | H | H | H | H | D | H | D | D | D | H |
| 295. | D | D | H | H | H | H | D | H | D | D | D | H |
| 296. | D | H | D | H | H | H | D | H | D | D | D | H |
| 297. | D | H | H | D | H | H | D | H | D | D | D | H |
| 298. | H | D | D | H | H | H | D | H | D | D | D | H |
| 299. | H | H | D | D | H | H | D | H | D | D | D | H |
| 300. | D | D | D | H | H | H | D | H | D | D | D | H |
| 301. | D | D | H | D | H | H | D | H | D | D | D | H |
| 302. | D | D | D | D | H | H | D | H | D | D | D | H |
| 303. | H | H | H | H | H | H | D | H | D | D | H | D |
| 304. | D | H | H | H | H | H | D | H | D | D | H | D |
| 305. | H | D | H | H | H | H | D | H | D | D | H | D |
| 306. | D | D | H | H | H | H | D | H | D | D | H | D |
| 307. | D | H | D | H | H | H | D | H | D | D | H | D |
| 308. | D | H | H | D | H | H | D | H | D | D | H | D |
| 309. | H | D | D | H | H | H | D | H | D | D | H | D |
| 310. | H | H | D | D | H | H | D | H | D | D | H | D |
| 311. | D | D | D | H | H | H | D | H | D | D | H | D |
| 312. | D | D | H | D | H | H | D | H | D | D | H | D |
| 313. | D | D | D | D | H | H | D | H | D | D | H | D |
| 314. | H | H | H | H | H | H | H | H | D | D | D | D |
| 315. | D | H | H | H | H | H | H | H | D | D | D | D |
| 316. | H | D | H | H | H | H | H | H | D | D | D | D |
| 317. | D | D | H | H | H | H | H | H | D | D | D | D |
| 318. | D | H | D | H | H | H | H | H | D | D | D | D |
| 319. | D | H | H | D | H | H | H | H | D | D | D | D |
| 320. | H | D | D | H | H | H | H | H | D | D | D | D |
| 321. | H | H | D | D | H | H | H | H | D | D | D | D |
| 322. | D | D | D | H | H | H | H | H | D | D | D | D |
| 323. | D | D | H | D | H | H | H | H | D | D | D | D |
| 324. | D | D | D | D | H | H | H | H | D | D | D | D |

TABLE 6
Exemplary compounds of Formula (III-A), (III-B), (III-
C), (III-D), (III-E), (III-F), (III-G), or (III-H).

| Row | Y⁵ | ⁶ | Y⁷ | Y⁸ | Y⁹ | Y¹⁰ | Y¹¹ | Y¹² | Y¹³ | Y¹⁴ | Y¹⁵ | Y¹⁶ |
|---|---|---|---|---|---|---|---|---|---|---|---|---|
| 1. | H | H | H | H | D | D | D | D | D | H | H | H |
| 2. | D | H | H | H | D | D | D | D | D | H | H | H |
| 3. | H | D | H | H | D | D | D | D | D | H | H | H |
| 4. | D | D | H | H | D | D | D | D | D | H | H | H |
| 5. | D | H | D | H | D | D | D | D | D | H | H | H |
| 6. | D | H | H | D | D | D | D | D | D | H | H | H |
| 7. | H | D | D | H | D | D | D | D | D | H | H | H |
| 8. | H | H | D | D | D | D | D | D | D | H | H | H |
| 9. | D | D | D | H | D | D | D | D | D | H | H | H |
| 10. | D | D | H | D | D | D | D | D | D | H | H | H |
| 11. | H | D | D | D | D | D | D | D | D | H | H | H |
| 12. | D | H | D | D | D | D | D | D | D | H | H | H |
| 13. | D | D | D | D | D | D | D | D | D | H | H | H |
| 14. | H | H | H | H | D | D | D | D | H | H | D | H |
| 15. | D | H | H | H | D | D | D | D | H | H | D | H |
| 16. | H | D | H | H | D | D | D | D | H | H | D | H |
| 17. | D | D | H | H | D | D | D | D | H | H | D | H |
| 18. | D | H | D | H | D | D | D | D | H | H | D | H |
| 19. | D | H | H | D | D | D | D | D | H | H | D | H |
| 20. | H | D | D | H | D | D | D | D | H | H | D | H |
| 21. | H | H | D | D | D | D | D | D | H | H | D | H |
| 22. | D | D | D | H | D | D | D | D | H | H | D | H |
| 23. | D | D | H | D | D | D | D | D | H | H | D | H |
| 24. | H | D | D | D | D | D | D | D | H | H | D | H |
| 25. | D | H | D | D | D | D | D | D | H | H | D | H |
| 26. | D | D | D | D | D | D | D | D | H | H | D | H |
| 27. | H | H | H | H | D | D | D | D | H | H | H | D |
| 28. | D | H | H | H | D | D | D | D | H | H | H | D |
| 29. | H | D | H | H | D | D | D | D | H | H | H | D |
| 30. | D | D | H | H | D | D | D | D | H | H | H | D |
| 31. | D | H | D | H | D | D | D | D | H | H | H | D |
| 32. | D | H | H | D | D | D | D | D | H | H | H | D |
| 33. | H | D | D | H | D | D | D | D | H | H | H | D |
| 34. | H | H | D | D | D | D | D | D | H | H | H | D |
| 35. | D | D | D | H | D | D | D | D | H | H | H | D |
| 36. | D | D | H | D | D | D | D | D | H | H | H | D |
| 37. | H | D | D | D | D | D | D | D | H | H | H | D |
| 38. | D | H | D | D | D | D | D | D | H | H | H | D |
| 39. | D | D | D | D | D | D | D | D | H | H | H | D |
| 40. | H | H | H | H | D | D | D | H | D | H | D | H |
| 41. | D | H | H | H | D | D | D | H | D | H | D | H |
| 42. | H | D | H | H | D | D | D | H | D | H | D | H |
| 43. | D | D | H | H | D | D | D | H | D | H | D | H |
| 44. | D | H | D | H | D | D | D | H | D | H | D | H |
| 45. | D | H | H | D | D | D | D | H | D | H | D | H |
| 46. | H | D | D | H | D | D | D | H | D | H | D | H |
| 47. | H | H | D | D | D | D | D | H | D | H | D | H |
| 48. | D | D | D | H | D | D | D | H | D | H | D | H |
| 49. | D | D | H | D | D | D | D | H | D | H | D | H |
| 50. | H | D | D | D | D | D | D | H | D | H | D | H |
| 51. | D | H | D | D | D | D | D | H | D | H | D | H |
| 52. | D | D | D | D | D | D | D | H | D | H | D | H |
| 53. | H | H | H | H | D | D | D | H | D | H | H | D |
| 54. | D | H | H | H | D | D | D | H | D | H | H | D |
| 55. | H | D | H | H | D | D | D | H | D | H | H | D |
| 56. | D | D | H | H | D | D | D | H | D | H | H | D |
| 57. | D | H | D | H | D | D | D | H | D | H | H | D |
| 58. | D | H | H | D | D | D | D | H | D | H | H | D |
| 59. | H | D | D | H | D | D | D | H | D | H | H | D |
| 60. | H | H | D | D | D | D | D | H | D | H | H | D |
| 61. | D | D | D | H | D | D | D | H | D | H | H | D |
| 62. | D | D | H | D | D | D | D | H | D | H | H | D |
| 63. | H | D | D | D | D | D | D | H | D | H | H | D |
| 64. | D | H | D | D | D | D | D | H | D | H | H | D |
| 65. | D | D | D | D | D | D | D | H | D | H | H | D |
| 66. | H | H | H | H | D | D | H | H | D | D | D | H |
| 67. | D | H | H | H | D | D | H | H | D | D | D | H |
| 68. | H | D | H | H | D | D | H | H | D | D | D | H |
| 69. | D | D | H | H | D | D | H | H | D | D | D | H |
| 70. | D | H | D | H | D | D | H | H | D | D | D | H |
| 71. | D | H | H | D | D | D | H | H | D | D | D | H |
| 72. | H | D | D | H | D | D | H | H | D | D | D | H |
| 73. | H | H | D | D | D | D | H | H | D | D | D | H |
| 74. | D | D | D | H | D | D | H | H | D | D | D | H |
| 75. | D | D | H | D | D | D | H | H | D | D | D | H |
| 76. | H | D | D | D | D | D | H | H | D | D | D | H |
| 77. | D | H | D | D | D | D | H | H | D | D | D | H |
| 78. | D | D | D | D | D | D | H | H | D | D | D | H |
| 79. | H | H | H | H | D | D | H | H | D | D | H | D |
| 80. | D | H | H | H | D | D | H | H | D | D | H | D |
| 81. | H | D | H | H | D | D | H | H | D | D | H | D |
| 82. | D | D | H | H | D | D | H | H | D | D | H | D |
| 83. | D | H | D | H | D | D | H | H | D | D | H | D |
| 84. | D | H | H | D | D | D | H | H | D | D | H | D |
| 85. | H | D | D | H | D | D | H | H | D | D | H | D |
| 86. | H | H | D | D | D | D | H | H | D | D | H | D |
| 87. | D | D | D | H | D | D | H | H | D | D | H | D |
| 88. | D | D | H | D | D | D | H | H | D | D | H | D |
| 89. | H | D | D | D | D | D | H | H | D | D | H | D |
| 90. | D | H | D | D | D | D | H | H | D | D | H | D |
| 91. | D | D | D | D | D | D | H | H | D | D | H | D |
| 92. | H | H | H | H | D | H | D | D | D | D | H | H |
| 93. | D | H | H | H | D | H | D | D | D | D | H | H |
| 94. | H | D | H | H | D | H | D | D | D | D | H | H |
| 95. | D | D | H | H | D | H | D | D | D | D | H | H |
| 96. | D | H | D | H | D | H | D | D | D | D | H | H |
| 97. | D | H | H | D | D | H | D | D | D | D | H | H |
| 98. | H | D | D | H | D | H | D | D | D | D | H | H |
| 99. | H | H | D | D | D | H | D | D | D | D | H | H |
| 100. | D | D | D | H | D | H | D | D | D | D | H | H |
| 101. | D | D | H | D | D | H | D | D | D | D | H | H |
| 102. | H | D | D | D | D | H | D | D | D | D | H | H |
| 103. | D | H | D | D | D | H | D | D | D | D | H | H |
| 104. | D | D | D | D | D | H | D | D | D | D | H | H |
| 105. | H | H | H | H | D | H | D | D | D | H | D | H |
| 106. | D | H | H | H | D | H | D | D | D | H | D | H |
| 107. | H | D | H | H | D | H | D | D | D | H | D | H |
| 108. | D | D | H | H | D | H | D | D | D | H | D | H |
| 109. | D | H | D | H | D | H | D | D | D | H | D | H |
| 110. | D | H | H | D | D | H | D | D | D | H | D | H |
| 111. | H | D | D | H | D | H | D | D | D | H | D | H |
| 112. | H | H | D | D | D | H | D | D | D | H | D | H |
| 113. | D | D | D | H | D | H | D | D | D | H | D | H |
| 114. | D | D | H | D | D | H | D | D | D | H | D | H |
| 115. | H | D | D | D | D | H | D | D | D | H | D | H |
| 116. | D | H | D | D | D | H | D | D | D | H | D | H |
| 117. | D | D | D | D | D | H | D | D | D | H | D | H |
| 118. | H | H | H | H | D | H | D | D | D | H | H | D |
| 119. | D | H | H | H | D | H | D | D | D | H | H | D |
| 120. | H | D | H | H | D | H | D | D | D | H | H | D |
| 121. | D | D | H | H | D | H | D | D | D | H | H | D |
| 122. | D | H | D | H | D | H | D | D | D | H | H | D |
| 123. | D | H | H | D | D | H | D | D | D | H | H | D |
| 124. | H | D | D | H | D | H | D | D | D | H | H | D |
| 125. | H | H | D | D | D | H | D | D | D | H | H | D |
| 126. | D | D | D | H | D | H | D | D | D | H | H | D |
| 127. | D | D | H | D | D | H | D | D | D | H | H | D |
| 128. | H | D | D | D | D | H | D | D | D | H | H | D |
| 129. | D | H | D | D | D | H | D | D | D | H | H | D |
| 130. | D | D | D | D | D | H | D | D | D | H | H | D |
| 131. | H | H | H | H | D | H | D | H | D | D | D | H |
| 132. | D | H | H | H | D | H | D | H | D | D | D | H |
| 133. | H | D | H | H | D | H | D | H | D | D | D | H |
| 134. | D | D | H | H | D | H | D | H | D | D | D | H |
| 135. | D | H | D | H | D | H | D | H | D | D | D | H |
| 136. | D | H | H | D | D | H | D | H | D | D | D | H |
| 137. | H | D | D | H | D | H | D | H | D | D | D | H |
| 138. | H | H | D | D | D | H | D | H | D | D | D | H |
| 139. | D | D | D | H | D | H | D | H | D | D | D | H |
| 140. | D | D | H | D | D | H | D | H | D | D | D | H |
| 141. | H | D | D | D | D | H | D | H | D | D | D | H |
| 142. | D | H | D | D | D | H | D | H | D | D | D | H |
| 143. | D | D | D | D | D | H | D | H | D | D | D | H |
| 144. | H | H | H | H | D | H | D | H | D | D | H | D |
| 145. | D | H | H | H | D | H | D | H | D | D | H | D |
| 146. | H | D | H | H | D | H | D | H | D | D | H | D |
| 147. | D | D | H | H | D | H | D | H | D | D | H | D |
| 148. | D | H | D | H | D | H | D | H | D | D | H | D |
| 149. | D | H | H | D | D | H | D | H | D | D | H | D |
| 150. | H | D | D | H | D | H | D | H | D | D | H | D |
| 151. | H | H | D | D | D | H | D | H | D | D | H | D |
| 152. | D | D | D | H | D | H | D | H | D | D | H | D |
| 153. | D | D | H | D | D | H | D | H | D | D | H | D |
| 154. | H | D | D | D | D | H | D | H | D | D | H | D |
| 155. | D | H | D | D | D | H | D | H | D | D | H | D |
| 156. | D | D | D | D | D | H | D | H | D | D | H | D |
| 157. | H | H | H | H | D | H | D | H | H | D | D | D |
| 158. | D | H | H | H | D | H | D | H | H | D | D | D |
| 159. | H | D | H | H | D | H | D | H | H | D | D | D |
| 160. | D | D | H | H | D | H | D | H | H | D | D | D |
| 161. | D | H | D | H | D | H | D | H | H | D | D | D |
| 162. | D | H | H | D | D | H | D | H | H | D | D | D |
| 163. | H | D | D | H | D | H | D | H | H | D | D | D |
| 164. | H | H | D | D | D | H | D | H | H | D | D | D |
| 165. | D | D | D | H | D | H | D | H | H | D | D | D |
| 166. | D | D | H | D | D | H | D | H | H | D | D | D |
| 167. | H | D | D | D | D | H | D | H | H | D | D | D |
| 168. | D | H | D | D | D | H | D | H | H | D | D | D |
| 169. | D | D | D | D | D | H | D | H | H | D | D | D |
| 170. | H | H | H | H | H | H | D | D | D | D | D | H |
| 171. | D | H | H | H | H | H | D | D | D | D | D | H |
| 172. | H | D | H | H | H | H | D | D | D | D | D | H |
| 173. | D | D | H | H | H | H | D | D | D | D | D | H |
| 174. | D | H | D | H | H | H | D | D | D | D | D | H |
| 175. | D | H | H | D | H | H | D | D | D | D | D | H |
| 176. | H | D | D | H | H | H | D | D | D | D | D | H |
| 177. | H | H | D | D | H | H | D | D | D | D | D | H |
| 178. | D | D | D | H | H | H | D | D | D | D | D | H |
| 179. | D | D | H | D | H | H | D | D | D | D | D | H |
| 180. | H | D | D | D | H | H | D | D | D | D | D | H |
| 181. | D | H | D | D | H | H | D | D | D | D | D | H |
| 182. | D | D | D | D | H | H | D | D | D | D | D | H |
| 183. | H | H | H | H | H | H | D | D | D | D | H | D |
| 184. | D | H | H | H | H | H | D | D | D | D | H | D |
| 185. | H | D | H | H | H | H | D | D | D | D | H | D |
| 186. | D | D | H | H | H | H | D | D | D | D | H | D |
| 187. | D | H | D | H | H | H | D | D | D | D | H | D |
| 188. | D | H | H | D | H | H | D | D | D | D | H | D |
| 189. | H | D | D | H | H | H | D | D | D | D | H | D |
| 190. | H | H | D | D | H | H | D | D | D | D | H | D |
| 191. | D | D | D | H | H | H | D | D | D | D | H | D |
| 192. | D | D | H | D | H | H | D | D | D | D | H | D |
| 193. | H | D | D | D | H | H | D | D | D | D | H | D |
| 194. | D | H | D | D | H | H | D | D | D | D | H | D |
| 195. | D | D | D | D | H | H | D | D | D | D | H | D |
| 196. | H | H | H | H | H | H | D | H | D | D | D | D |
| 197. | D | H | H | H | H | H | D | H | D | D | D | D |
| 198. | H | D | H | H | H | H | D | H | D | D | D | D |
| 199. | D | D | H | H | H | H | D | H | D | D | D | D |
| 200. | D | H | D | H | H | H | D | H | D | D | D | D |
| 201. | D | H | H | D | H | H | D | H | D | D | D | D |
| 202. | H | D | D | H | H | H | D | H | D | D | D | D |
| 203. | H | H | D | D | H | H | D | H | D | D | D | D |
| 204. | D | D | D | H | H | H | D | H | D | D | D | D |
| 205. | D | D | H | D | H | H | D | H | D | D | D | D |
| 206. | H | D | D | D | H | H | D | H | D | D | D | D |
| 207. | D | H | D | D | H | H | D | H | D | D | D | D |
| 208. | D | D | D | D | H | H | D | H | D | D | D | D |
| 209. | H | H | H | H | D | D | D | D | D | D | H | H |
| 210. | D | H | H | H | D | D | D | D | D | D | H | H |
| 211. | H | D | H | H | D | D | D | D | D | D | H | H |
| 212. | D | D | H | H | D | D | D | D | D | D | H | H |
| 213. | D | H | D | H | D | D | D | D | D | D | H | H |
| 214. | D | H | H | D | D | D | D | D | D | D | H | H |
| 215. | H | D | D | H | D | D | D | D | D | D | H | H |
| 216. | H | H | D | D | D | D | D | D | D | D | H | H |
| 217. | D | D | D | H | D | D | D | D | D | D | H | H |
| 218. | D | D | H | D | D | D | D | D | D | D | H | H |
| 219. | H | D | D | D | D | D | D | D | D | D | H | H |
| 220. | D | H | D | D | D | D | D | D | D | D | H | H |
| 221. | D | D | D | D | D | D | D | D | D | D | H | H |
| 222. | H | H | H | H | D | D | D | D | D | H | D | H |
| 223. | D | H | H | H | D | D | D | D | D | H | D | H |
| 224. | H | D | H | H | D | D | D | D | D | H | D | H |
| 225. | D | D | H | H | D | D | D | D | D | H | D | H |
| 226. | D | H | D | H | D | D | D | D | D | H | D | H |
| 227. | D | H | H | D | D | D | D | D | D | H | D | H |
| 228. | H | D | D | H | D | D | D | D | D | H | D | H |
| 229. | H | H | D | D | D | D | D | D | D | H | D | H |
| 230. | D | D | D | H | D | D | D | D | D | H | D | H |
| 231. | D | D | H | D | D | D | D | D | D | H | D | H |
| 232. | H | D | D | D | D | D | D | D | D | H | D | H |
| 233. | D | H | D | D | D | D | D | D | D | H | D | H |
| 234. | D | D | D | D | D | D | D | D | D | H | D | H |
| 235. | H | H | H | H | D | D | D | D | D | H | H | D |
| 236. | D | H | H | H | D | D | D | D | D | H | H | D |
| 237. | H | D | H | H | D | D | D | D | D | H | H | D |
| 238. | D | D | H | H | D | D | D | D | D | H | H | D |
| 239. | D | H | D | H | D | D | D | D | D | H | H | D |
| 240. | D | H | H | D | D | D | D | D | D | H | H | D |
| 241. | H | D | D | H | D | D | D | D | D | H | H | D |
| 242. | H | H | D | D | D | D | D | D | D | H | H | D |
| 243. | D | D | D | H | D | D | D | D | D | H | H | D |
| 244. | D | D | H | D | D | D | D | D | D | H | H | D |
| 245. | H | D | D | D | D | D | D | D | D | H | H | D |
| 246. | D | H | D | D | D | D | D | D | D | H | H | D |
| 247. | D | D | D | D | D | D | D | D | D | H | H | D |
| 248. | H | H | H | H | D | D | D | D | H | H | D | D |
| 249. | D | H | H | H | D | D | D | D | H | H | D | D |
| 250. | H | D | H | H | D | D | D | D | H | H | D | D |
| 251. | D | D | H | H | D | D | D | D | H | H | D | D |
| 252. | D | H | D | H | D | D | D | D | H | H | D | D |
| 253. | D | H | H | D | D | D | D | D | H | H | D | D |
| 254. | H | D | D | H | D | D | D | D | H | H | D | D |
| 255. | H | H | D | D | D | D | D | D | H | H | D | D |
| 256. | D | D | D | H | D | D | D | D | H | H | D | D |
| 257. | D | D | H | D | D | D | D | D | H | H | D | D |
| 258. | H | D | D | D | D | D | D | D | H | H | D | D |
| 259. | D | H | D | D | D | D | D | D | H | H | D | D |
| 260. | D | D | D | D | D | D | D | D | H | H | D | D |
| 261. | H | H | H | H | D | H | D | D | D | D | D | H |
| 262. | D | H | H | H | D | H | D | D | D | D | D | H |
| 263. | H | D | H | H | D | H | D | D | D | D | D | H |
| 264. | D | D | H | H | D | H | D | D | D | D | D | H |
| 265. | D | H | D | H | D | H | D | D | D | D | D | H |
| 266. | D | H | H | D | D | H | D | D | D | D | D | H |
| 267. | H | D | D | H | D | H | D | D | D | D | D | H |
| 268. | H | H | D | D | D | H | D | D | D | D | D | H |
| 269. | D | D | D | H | D | H | D | D | D | D | D | H |
| 270. | D | D | H | D | D | H | D | D | D | D | D | H |
| 271. | H | D | D | D | D | H | D | D | D | D | D | H |
| 272. | D | H | D | D | D | H | D | D | D | D | D | H |
| 273. | D | D | D | D | D | H | D | D | D | D | D | H |
| 274. | H | H | H | H | D | H | D | D | D | D | H | D |
| 275. | D | H | H | H | D | H | D | D | D | D | H | D |
| 276. | H | D | H | H | D | H | D | D | D | D | H | D |
| 277. | D | D | H | H | D | H | D | D | D | D | H | D |
| 278. | D | H | D | H | D | H | D | D | D | D | H | D |
| 279. | D | H | H | D | D | H | D | D | D | D | H | D |
| 280. | H | D | D | H | D | H | D | D | D | D | H | D |
| 281. | H | H | D | D | D | H | D | D | D | D | H | D |
| 282. | D | D | D | H | D | H | D | D | D | D | H | D |
| 283. | D | D | H | D | D | H | D | D | D | D | H | D |
| 284. | H | D | D | D | D | H | D | D | D | D | H | D |
| 285. | D | H | D | D | D | H | D | D | D | D | H | D |
| 286. | D | D | D | D | D | H | D | D | D | D | H | D |
| 287. | H | H | H | H | D | H | D | D | H | D | D | D |
| 288. | D | H | H | H | D | H | D | D | H | D | D | D |
| 289. | H | D | H | H | D | H | D | D | H | D | D | D |
| 290. | D | D | H | H | D | H | D | D | H | D | D | D |
| 291. | D | H | D | H | D | H | D | D | H | D | D | D |
| 292. | D | H | H | D | D | H | D | D | H | D | D | D |
| 293. | H | D | D | H | D | H | D | D | H | D | D | D |
| 294. | H | H | D | D | D | H | D | D | H | D | D | D |
| 295. | D | D | D | H | D | H | D | D | H | D | D | D |
| 296. | D | D | H | D | D | H | D | D | H | D | D | D |
| 297. | H | D | D | D | D | H | D | D | H | D | D | D |
| 298. | D | H | D | D | D | H | D | D | H | D | D | D |
| 299. | D | D | D | D | D | H | D | D | H | D | D | D |
| 300. | H | H | H | H | D | H | D | H | D | D | D | D |
| 301. | D | H | H | H | D | H | D | H | D | D | D | D |
| 302. | H | D | H | H | D | H | D | H | D | D | D | D |
| 303. | D | D | H | H | D | H | D | H | D | D | D | D |
| 304. | D | H | D | H | D | H | D | H | D | D | D | D |
| 305. | D | H | H | D | D | H | D | H | D | D | D | D |
| 306. | H | D | D | H | D | H | D | H | D | D | D | D |
| 307. | H | H | D | D | D | H | D | H | D | D | D | D |
| 308. | D | D | D | H | D | H | D | H | D | D | D | D |
| 309. | D | D | H | D | D | H | D | H | D | D | D | D |
| 310. | H | D | D | D | D | H | D | H | D | D | D | D |
| 311. | D | H | D | D | D | H | D | H | D | D | D | D |
| 312. | D | D | D | D | D | H | D | H | D | D | D | D |
| 313. | H | H | H | H | H | H | D | D | D | D | D | D |
| 314. | D | H | H | H | H | H | D | D | D | D | D | D |
| 315. | H | D | H | H | H | H | D | D | D | D | D | D |
| 316. | D | D | H | H | H | H | D | D | D | D | D | D |
| 317. | D | H | D | H | H | H | D | D | D | D | D | D |
| 318. | D | H | H | D | H | H | D | D | D | D | D | D |
| 319. | H | D | D | H | H | H | D | D | D | D | D | D |
| 320. | H | H | D | D | H | H | D | D | D | D | D | D |
| 321. | D | D | D | H | H | H | D | D | D | D | D | D |
| 322. | D | D | H | D | H | H | D | D | D | D | D | D |
| 323. | H | D | D | D | H | H | D | D | D | D | D | D |
| 324. | D | H | D | D | H | H | D | D | D | D | D | D |
| 325. | D | D | D | D | H | H | D | D | D | D | D | D |
| 326. | H | H | H | H | D | D | D | D | D | D | D | H |
| 327. | D | H | H | H | D | D | D | D | D | D | D | H |
| 328. | H | D | H | H | D | D | D | D | D | D | D | H |
| 329. | D | D | H | H | D | D | D | D | D | D | D | H |
| 330. | D | H | D | H | D | D | D | D | D | D | D | H |
| 331. | D | H | H | D | D | D | D | D | D | D | D | H |
| 332. | H | D | D | H | D | D | D | D | D | D | D | H |
| 333. | H | H | D | D | D | D | D | D | D | D | D | H |
| 334. | D | D | D | H | D | D | D | D | D | D | D | H |
| 335. | D | D | H | D | D | D | D | D | D | D | D | H |
| 336. | H | D | D | D | D | D | D | D | D | D | D | H |
| 337. | D | H | D | D | D | D | D | D | D | D | D | H |
| 338. | D | D | D | D | D | D | D | D | D | D | D | H |
| 339. | H | H | H | H | D | D | D | D | D | D | H | D |
| 340. | D | H | H | H | D | D | D | D | D | D | H | D |
| 341. | H | D | H | H | D | D | D | D | D | D | H | D |
| 342. | D | D | H | H | D | D | D | D | D | D | H | D |
| 343. | D | H | D | H | D | D | D | D | D | D | H | D |
| 344. | D | H | H | D | D | D | D | D | D | D | H | D |
| 345. | H | D | D | H | D | D | D | D | D | D | H | D |
| 346. | H | H | D | D | D | D | D | D | D | D | H | D |
| 347. | D | D | D | H | D | D | D | D | D | D | H | D |
| 348. | D | D | H | D | D | D | D | D | D | D | H | D |
| 349. | H | D | D | D | D | D | D | D | D | D | H | D |
| 350. | D | H | D | D | D | D | D | D | D | D | H | D |
| 351. | D | D | D | D | D | D | D | D | D | D | H | D |
| 352. | H | H | H | H | D | H | D | D | D | D | D | D |
| 353. | D | H | H | H | D | H | D | D | D | D | D | D |
| 354. | H | D | H | H | D | H | D | D | D | D | D | D |
| 355. | D | D | H | H | D | H | D | D | D | D | D | D |
| 356. | D | H | D | H | D | H | D | D | D | D | D | D |
| 357. | D | H | H | D | D | H | D | D | D | D | D | D |
| 358. | H | D | D | H | D | H | D | D | D | D | D | D |
| 359. | H | H | D | D | D | H | D | D | D | D | D | D |
| 360. | D | D | D | H | D | H | D | D | D | D | D | D |
| 361. | D | D | H | D | D | H | D | D | D | D | D | D |
| 362. | H | D | D | D | D | H | D | D | D | D | D | D |
| 363. | D | H | D | D | D | H | D | D | D | D | D | D |
| 364. | D | D | D | D | D | H | D | D | D | D | D | D |
| 365. | H | H | H | H | D | D | D | H | D | D | D | D |
| 366. | D | H | H | H | D | D | D | H | D | D | D | D |
| 367. | H | D | H | H | D | D | D | H | D | D | D | D |
| 368. | D | D | H | H | D | D | D | H | D | D | D | D |
| 369. | D | H | D | H | D | D | D | H | D | D | D | D |
| 370. | D | H | H | D | D | D | D | H | D | D | D | D |
| 371. | H | D | D | H | D | D | D | H | D | D | D | D |
| 372. | H | H | D | D | D | D | D | H | D | D | D | D |
| 373. | D | D | D | H | D | D | D | H | D | D | D | D |
| 374. | D | D | H | D | D | D | D | H | D | D | D | D |
| 375. | H | D | D | D | D | D | D | H | D | D | D | D |
| 376. | D | H | D | D | D | D | D | H | D | D | D | D |
| 377. | D | D | D | D | D | D | D | H | D | D | D | D |
| 378. | H | H | H | H | D | D | D | D | D | H | D | D |
| 379. | D | H | H | H | D | D | D | D | D | H | D | D |
| 380. | H | D | H | H | D | D | D | D | D | H | D | D |
| 381. | D | D | H | H | D | D | D | D | D | H | D | D |
| 382. | D | H | D | H | D | D | D | D | D | H | D | D |
| 383. | D | H | H | D | D | D | D | D | D | H | D | D |
| 384. | H | D | D | H | D | D | D | D | D | H | D | D |
| 385. | H | H | D | D | D | D | D | D | D | H | D | D |
| 386. | D | D | D | H | D | D | D | D | D | H | D | D |
| 387. | D | D | H | D | D | D | D | D | D | H | D | D |
| 388. | H | D | D | D | D | D | D | D | D | H | D | D |
| 389. | D | H | D | D | D | D | D | D | D | H | D | D |
| 390. | D | D | D | D | D | D | D | D | D | H | D | D |
| 391. | H | H | H | H | D | D | D | D | D | D | D | D |
| 392. | D | H | H | H | D | D | D | D | D | D | D | D |
| 393. | H | D | H | H | D | D | D | D | D | D | D | D |
| 394. | D | D | H | H | D | D | D | D | D | D | D | D |
| 395. | D | H | D | H | D | D | D | D | D | D | D | D |
| 396. | D | H | H | D | D | D | D | D | D | D | D | D |
| 397. | H | D | D | H | D | D | D | D | D | D | D | D |
| 398. | H | H | D | D | D | D | D | D | D | D | D | D |
| 399. | D | D | D | H | D | D | D | D | D | D | D | D |
| 400. | D | D | H | D | D | D | D | D | D | D | D | D |
| 401. | H | D | D | D | D | D | D | D | D | D | D | D |
| 402. | D | H | D | D | D | D | D | D | D | D | D | D |
| 403. | D | D | D | D | D | D | D | D | D | D | D | D |

In some embodiments, the compounds provided herein, including any compound specifically provided in the tables above, have a diastereomeric excess (de) of at least about 1%, about 5%, about 10%, about 20%, about 30%, about 40%, about 50%, about 75%, about 90%, about 95%, or about 99%. In some embodiments, the diastereomeric excess is at least 50%. In some embodiments, the diastereomeric excess is at least 75%. In some embodiments, the diastereomeric excess is at least 90%. In some embodiments, the diastereomeric excess is at least 95%. In some embodiments, the diastereomeric excess is at least 99%.

In some embodiments, the compounds provided herein, including any compound specifically provided in the tables above, have am enantiomeric excess (ee) of at least about 1%, about 5%, about 10%, about 20%, about 30%, about 40%, about 50%, about 75%, about 90%, about 95%, or about 99%. In some embodiments, the enantiomeric excess is at least 50%. In some embodiments, the enantiomeric excess is at least 75%. In some embodiments, the enantiomeric excess is at least 90%. In some embodiments, the enantiomeric excess is at least 95%. In some embodiments, the enantiomeric excess is at least 99%.

The compounds provided herein are often shown as having (3S,4R) stereochemistry in the tetrahydropyran moiety. In another set of embodiments, provided herein are the corresponding (3R, 4S), (1R, 2R) or (3S, 4S) compounds provided herein, including any compound specifically provided in the tables above. In another set of embodiments, provided herein are the corresponding mixtures of the compounds provided herein, including any compound specifically provided in the tables above.

In some embodiments, for the compounds provided herein, including any compound specifically provided in the tables above, each position designated as deuterium independently has a minimum isotopic enrichment factor of at least 1000 (15% deuterium incorporation), at least 2000 (30% deuterium incorporation), at least 3000 (45% deuterium incorporation), at least 3500 (52.5% deuterium incorporation), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation) at each designated deuterium atom.

The synthesis of the compounds provided herein may be readily achieved by synthetic chemists of ordinary skill by reference to the exemplary synthesis and examples provided herein. Analogous procedures used for the preparation of certain non-deuterium enriched aminoheteroaryl kinase inhibitors are provided in International Application Publication Nos. WO 2023/093769 and WO 2022/111621, the entireties of which are incorporated herein by reference.

### Pharmaceutical Compositions

Certain embodiments are directed to a pharmaceutical composition comprising one or more compounds described herein.

In some embodiments, the pharmaceutical composition can optionally contain one or more pharmaceutically acceptable excipients. In some embodiments, the pharmaceutical composition comprises a compound described herein (e.g., any of the specific compounds described in Tables 1-6 herein, or a pharmaceutically acceptable salt thereof) and one or more pharmaceutically acceptable excipients. Pharmaceutically acceptable excipients are known in the art. Non-limiting suitable excipients include, for example, encapsulating materials or additives such as antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. See also Remington's The Science and Practice of Pharmacy, 21st Edition, A. R. Gennaro (Lippincott, Williams & Wilkins, Baltimore, Md., 2005; incorporated herein by reference), which describes various excipients used in formulating pharmaceutical compositions and known techniques for the preparation thereof.

The pharmaceutical composition can include any one or more of the compounds described herein. For example, in some embodiments, the pharmaceutical composition comprises any of any of the specific compounds described in Tables 1-6 herein, or a pharmaceutically acceptable salt thereof, e.g., in a therapeutically effective amount. In any of the embodiments described herein, the pharmaceutical composition can comprise a therapeutically effective amount (e.g., for treating breast cancer or ovarian cancer) of a compound selected from any the specific compounds described in Tables 1-6 herein, or a pharmaceutically acceptable salt thereof. In some embodiments, the pharmaceutical composition can comprise a compound selected from the compounds according to those in Tables 1-6 herein that have a CDK2/CyclinE1 IC50 level of less than 100 nM, or less than 10 nM, when tested according to Biological Example 1 as described in WO2022/111621, the entirely of which is incorporated herein by reference.

The pharmaceutical composition herein can be formulated for delivery via any of the known routes of delivery, which include but not limited to administering orally, nasally, transdermally, pulmonary, inhalationally, buccally, sublingually, intraperintoneally, subcutaneously, intramuscularly, intravenously, rectally, intrapleurally, intrathecally or parenterally.

In some embodiments, the pharmaceutical composition can be formulated for oral administration. The oral formulations can be presented in discrete units, such as capsules, pills, cachets, lozenges, or tablets, each containing a predetermined amount of the active compound; as a powder or granules; as a solution or a suspension in an aqueous or non-aqueous liquid; or as an oil-in-water or water-in-oil emulsion. Excipients for the preparation of compositions for oral administration are known in the art. Non-limiting suitable excipients include, for example, agar, alginic acid, aluminum hydroxide, benzyl alcohol, benzyl benzoate, 1, 3-butylene glycol, carbomers, castor oil, cellulose, cellulose acetate, cocoa butter, corn starch, corn oil, cottonseed oil, cross-povidone, diglycerides, ethanol, ethyl cellulose, ethyl laureate, ethyl oleate, fatty acid esters, gelatin, germ oil, glucose, glycerol, groundnut oil, hydroxypropylmethyl cellulose, isopropanol, isotonic saline, lactose, magnesium hydroxide, magnesium stearate, malt, mannitol, monoglycerides, olive oil, peanut oil, potassium phosphate salts, potato starch, povidone, propylene glycol, Ringer's solution, safflower oil, sesame oil, sodium carboxymethyl cellulose, sodium phosphate salts, sodium lauryl sulfate, sodium sorbitol, soybean oil, stearic acids, stearyl fumarate, sucrose, surfactants, talc, tragacanth, tetrahydrofurfuryl alcohol, triglycerides, water, and mixtures thereof.

In some embodiments, the pharmaceutical composition is formulated for parenteral administration (such as intravenous injection or infusion, subcutaneous or intramuscular injection). The parenteral formulations can be, for example, an aqueous solution, a suspension, or an emulsion. Excipients for the preparation of parenteral formulations are known in the art. Non-limiting suitable excipients include, for example, 1, 3-butanediol, castor oil, corn oil, cottonseed oil, dextrose, germ oil, groundnut oil, liposomes, oleic acid, olive oil, peanut oil, Ringer's solution, safflower oil, sesame oil, soybean oil, U.S.P. or isotonic sodium chloride solution, water and mixtures thereof.

Compounds of the present application can be used alone, in combination with each other, or in combination with one or more additional therapeutic agents, e.g., in combination with an additional anticancer therapeutic agent, such as mitotic inhibitors, alkylating agents, antimetabolites, antitumor antibiotics, anti-angiogenesis agents, topoisomerase I and II inhibitors, plant alkaloids, hormonal agents and antagonists, growth factor inhibitors, radiation, signal transduction inhibitors, such as inhibitors of protein tyrosine kinases and/or serine/threonine kinases, cell cycle inhibitors, biological response modifiers, enzyme inhibitors, antisense oligonucleotides or oligonucleotide derivatives, cytotoxics, immuno-oncology agents, and the like. In some embodiments, one or more compounds of the present application can be used in combination with one or more targeted agents, such as inhibitors of PI3 kinase, mTOR, PARP, IDO, TDO, ALK, ROS, MEK, VEGF, FLT3, AXL, ROR2, EGFR, FGFR, Src/Abl, RTK/Ras, Myc, Raf, PDGF, AKT, c-Kit, erbB, CDK4/CDK6, CDK5, CDK7, CDK9, SMO, CXCR4, HER2, GLS1, EZH2 or Hsp90, or immunomodulatory agents, such as PD-1 or PD-L1 antagonists, OX40 agonists or 4-1BB agonists. In some embodiments, one or more compounds of the present application can be used in combination with a standard of care agent, such as tamoxifen, docetaxel, paclitaxel, cisplatin, capecitabine, gemcitabine, vinorelbine, exemestane, letrozole, fulvestrant, anastrozole or trastuzumab. Suitable additional anticancer therapeutic agents include any of those known in the art, such as those approved for the appropriate cancer by a regulatory agency such as the U.S. Food and Drug Administration. Some examples of suitable additional anticancer therapeutic agents also include those described in WO2020/157652, US2018/0044344, WO2008/122767, etc., the content of each of which is herein incorporated by reference in its entireties.

When used in combination with one or more additional therapeutic agents, compounds of the present application or pharmaceutical compositions herein can be administered to the subject either concurrently or sequentially in any order with such additional therapeutic agents. In some embodiments, the pharmaceutical composition can comprise one or more compounds of the present application and the one or more additional therapeutic agents in a single composition. In some embodiments, the pharmaceutical composition comprising one or more compounds of the present application can be included in a kit which also comprises a separate pharmaceutical composition comprising the one or more additional therapeutic agents.

The pharmaceutical composition can include various amounts of the compounds of the present application, depending on various factors such as the intended use and potency and selectivity of the compounds. In some embodiments, the pharmaceutical composition comprises a therapeutically effective amount of a compound of the present application. In some embodiments, the pharmaceutical composition comprises a therapeutically effective amount of the compound of the present application and a pharmaceutically acceptable excipient. As used herein, a therapeutically effective amount of a compound of the present application is an amount effective to treat a disease or disorder as described herein, such as breast cancer or ovarian cancer, which can depend on the recipient of the treatment, the disorder, condition or disease being treated and the severity thereof, the composition containing the compound, the time of administration, the route of administration, the duration of treatment, the compound potency, its rate of clearance and whether or not another drug is co-administered.

### Method of Treatment

Compounds of the present application have various utilities. For example, compounds of the present application can be used as therapeutic active substances for the treatment and/or prophylaxis of a CDK2-mediated disease or disorder. Accordingly, some embodiments of the present application are also directed to methods of using one or more compounds of the present application or pharmaceutical compositions herein for treating or preventing a CDK2-mediated disease or disorder in a subject in need thereof, such as for treating cancer in a subject in need thereof.

In some embodiments, the present application provides a method of inhibiting abnormal cell growth in a subject in need thereof, comprising administering to the subject a therapeutically effective amount of a compound of the present application or a pharmaceutical composition described herein. In some embodiments, the abnormal cell growth is cancer characterized by amplification or overexpression of cyclin E1 (CCNE1) and/or cyclin E2 (CCNE2). In some embodiments, the subject is identified as having a cancer characterized by amplification or overexpression of CCNE1 and/or CCNE2.

In some embodiments, the present application also provides a method of inhibiting CDK activity in a subject or biological sample. In some embodiments, the present application provides a method of inhibiting CDK2 activity in a subject or biological sample, which comprises contacting the subject or biological sample with an effective amount of the compound of the present application (e.g., any of the specific compounds disclosed in Tables 1-6 herein, or a pharmaceutically acceptable salt thereof) or a pharmaceutical composition described herein.

In some embodiments, the present application provides a method of treating or preventing a CDK mediated, in particular a CDK2-mediated, disease or disorder in a subject in need thereof. In some embodiments, the method comprises administering to the subject an effective amount of a compound of the present application (e.g., any of the specific compounds described in Tables 1-6 herein, or a pharmaceutically acceptable salt thereof) or an effective amount of a pharmaceutical composition described herein. In some embodiments, the CDK2-mediated disease or disorder is cancer. In some embodiments, the cancer is characterized by amplification or overexpression of CCNE1 and/or CCNE2.

In some embodiments, the present application also provides a method of treating or preventing cancer in a subject in need thereof, which comprises administering to the subject an effective amount of a compound of the present application (e.g., any of the specific compounds described in Tables 1-6 herein, or a pharmaceutically acceptable salt thereof) or an effective amount of a pharmaceutical composition described herein. In some embodiments, the cancer is characterized by amplification or overexpression of CCNE1 and/or CCNE2. In some embodiments, the subject is identified as having a cancer characterized by amplification or overexpression of CCNE1 and/or CCNE2. In some embodiments, the cancer is selected from breast cancer, ovarian cancer, bladder cancer, uterine cancer, prostate cancer, lung cancer (including NSCLC, SCLC, squamous cell carcinoma or adenocarcinoma), esophageal cancer, head and neck cancer, colorectal cancer, kidney cancer (including RCC), liver cancer (including HCC), pancreatic cancer, stomach (i.e., gastric) cancer, thyroid cancer, and combinations thereof. In some embodiments of the methods herein, the cancer is breast cancer, ovarian cancer, bladder cancer, uterine cancer, prostate cancer, lung cancer, esophageal cancer, liver cancer, pancreatic cancer and/or stomach cancer.

In some embodiments of the methods herein, the cancer is breast cancer, such as ER-positive/HR-positive, HER2-negative breast cancer; ER-positive/HR-positive, HER2-positive breast cancer; triple negative breast cancer (TNBC); or inflammatory breast cancer. In some embodiments, the breast cancer can be endocrine resistant breast cancer, trastuzumab resistant breast cancer, or breast cancer demonstrating primary or acquired resistance to CDK4/CDK6 inhibition. In some embodiments, the breast cancer can be advanced or metastatic breast cancer. In some embodiments, the breast cancer described herein is characterized by amplification or overexpression of CCNE1 and/or CCNE2.

In some embodiments of the methods herein, the cancer is ovarian cancer. In some embodiments, the ovarian cancer is characterized by amplification or overexpression of CCNE1 and/or CCNE2.

In some embodiments of the methods herein, the cancer is blood cancer such as leukemia. In some embodiments of the methods herein, the cancer is chronic lymphocytic leukemia, such as relapsed or refractory Chronic Lymphocytic Leukemia (CLL).

In some embodiments of the methods herein, the cancer is acute myeloid leukemia. In some embodiments of the methods herein, the cancer is relapsed or refractory Acute Myeloid Leukemia or Myelodysplastic Syndromes.

In any of the embodiments described herein, unless otherwise specified or contradictory, the cancer herein can be characterized by amplification or overexpression of CCNE1 and/or CCNE2.

In some embodiments, the present application also provides a method of treating breast cancer in a subject in need thereof, which comprises administering to the subject a therapeutically effective amount of a compound of the present application (e.g., any of the specific compounds described in Tables 1-6 herein, or a pharmaceutically acceptable salt thereof) or an effective amount of a pharmaceutical composition described herein. In some embodiments, the breast cancer is selected from ER-positive/HR-positive, HER2-negative breast cancer; ER-positive/HR-positive, HER2-positive breast cancer; triple negative breast cancer (TNBC); and inflammatory breast cancer. In some embodiments, the breast cancer is selected from endocrine resistant breast cancer, trastuzumab resistant breast cancer, or breast cancer demonstrating primary or acquired resistance to CDK4/CDK6 inhibition. In some embodiments, the breast cancer is advanced or metastatic breast cancer. In some embodiments, the breast cancer is characterized by amplification or overexpression of CCNE1 and/or CCNE2.

In some embodiments, the present application also provides a method of treating ovarian cancer in a subject in need thereof, which comprises administering to the subject a therapeutically effective amount of a compound of the present application (e.g., any of the specific compounds described in Tables 1-6 herein, or a pharmaceutically acceptable salt thereof) or an effective amount of a pharmaceutical composition described herein. In some embodiments, the ovarian cancer is characterized by amplification or overexpression of CCNE1 and/or CCNE2.

In some embodiments, the present application also provides a method of treating leukemia in a subject in need thereof, which comprises administering to the subject a therapeutically effective amount of a compound of the present application (e.g., any of the specific compounds described in Tables 1-6 herein, or a pharmaceutically acceptable salt thereof) or an effective amount of a pharmaceutical composition described herein. In some embodiments, the leukemia is characterized by amplification or overexpression of CCNE1 and/or CCNE2.

In some embodiments, the present application also provides a method of treating chronic lymphocytic leukemia, such as relapsed or refractory Chronic Lymphocytic Leukemia (CLL), in a subject in need thereof, which comprises administering to the subject a therapeutically effective amount of a compound of the present application (e.g., any of the specific compounds described in Tables 1-6 herein, or a pharmaceutically acceptable salt thereof) or an effective amount of a pharmaceutical composition described herein.

In some embodiments, the present application also provides a method of treating acute myeloid leukemia, such as relapsed or refractory Acute Myeloid Leukemia, in a subject in need thereof, which comprises administering to the subject a therapeutically effective amount of a compound of the present application (e.g., any of the specific compounds described in Tables 1-6 herein, or a pharmaceutically acceptable salt thereof) or an effective amount of a pharmaceutical composition described herein.

In some embodiments, the present application also provides a method of treating Myelodysplastic Syndromes in a subject in need thereof, which comprises administering to the subject a therapeutically effective amount of a compound of the present application (e.g., any of the specific compounds described in Tables 1-6 herein, or a pharmaceutically acceptable salt thereof) or an effective amount of a pharmaceutical composition described herein.

In some embodiments, the compound of the present application for the methods herein has a CDK2/CyclinE1 IC50 of less than 100 nM or less than 10 nM, measured/calculated according to the Biological Example 1 as described in WO2022/111621.

The administering in the methods herein is not limited to any particular route of administration. For example, in some embodiments, the administering can be orally, nasally, transdermally, pulmonary, inhalationally, buccally, sublingually, intraperintoneally, subcutaneously, intramuscularly, intravenously, rectally, intrapleurally, intrathecally and parenterally. In some embodiments, the administering is orally. In some embodiments, the administering is a parenteral injection, such as an intraveneous injection.

Compounds of the present application can be used as a monotherapy or in a combination therapy. In some embodiments according to the methods described herein, one or more compounds of the present application can be administered as the only active ingredient (s). In some embodiments according to the methods described herein, one or more compounds of the present application can also be co-administered with an additional therapeutic agent, either concurrently or sequentially in any order, to the subject in need thereof. The additional therapeutic agent can typically be an additional anticancer therapeutic agent, such as mitotic inhibitors, alkylating agents, antimetabolites, antitumor antibiotics, anti-angiogenesis agents, topoisomerase I and II inhibitors, plant alkaloids, hormonal agents and antagonists, growth factor inhibitors, radiation, signal transduction inhibitors, such as inhibitors of protein tyrosine kinases and/or serine/threonine kinases, cell cycle inhibitors, biological response modifiers, enzyme inhibitors, antisense oligonucleotides or oligonucleotide derivatives, cytotoxics, immuno-oncology agents, and the like. In some embodiments, the additional anticancer agent is an endocrine agent, such as an aromatase inhibitor, a SERD or a SERM. In some embodiments, one or more compounds of the present application can be administered in combination with one or more targeted agents, such as inhibitors of PI3 kinase, mTOR, PARP, IDO, TDO, ALK, ROS, MEK, VEGF, FLT3, AXL, ROR2, EGFR, FGFR, Src/Abl, RTK/Ras, Myc, Raf, PDGF, AKT, c-Kit, erbB, CDK4/CDK6, CDK5, CDK7, CDK9, SMO, CXCR4, HER2, GLS1, EZH2 or Hsp90, or immunomodulatory agents, such as PD-1 or PD-L1 antagonists, OX40 agonists or 4-1BB agonists. In some embodiments, one or more compounds of the present application can be administered in combination with a standard of care agent, such as tamoxifen, docetaxel, paclitaxel, cisplatin, capecitabine, gemcitabine, vinorelbine, exemestane, letrozole, fulvestrant, anastrozole or trastuzumab. Suitable additional anticancer therapeutic agent include any of those known in the art, such as those approved for the appropriate cancer by a regulatory agency such as the U.S. Food and Drug Administration. Some examples of suitable additional anticancer therapeutic agents also include those described in WO2020/157652, US2018/0044344, WO2008/122767, etc., the contents of each of which is incorporated by reference herein in their entirety.

Dosing regimen including doses for the methods described herein can vary and be adjusted, which can depend on the recipient of the treatment, the disorder, condition or disease being treated and the severity thereof, the composition containing the compound, the time of administration, the route of administration, the duration of treatment, the compound potency, its rate of clearance and whether or not another drug is co-administered.

### EXEMPLARY EMBODIMENTS

Embodiment I-1. A compound of Formula (I):

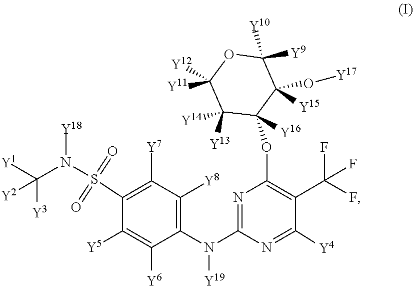

or a stereoisomer, a mixture of stereoisomers, tautomer, or a pharmaceutically acceptable salt thereof, wherein at least one of Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, Y¹⁶, Y¹⁷, Y¹⁸, and Y¹⁹ is a hydrogen that is isotopically enriched with deuterium (“D”), and the others of Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, Y¹⁶, Y¹⁷, Y¹⁸, and Y¹⁹ are non-enriched hydrogen atoms (“H”), provided that the compound is not

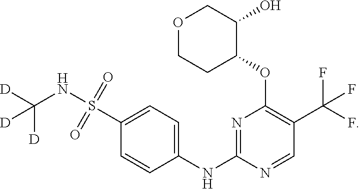 [SMILES: [2H]C([2H])([2H])NS(=O)(=O)c1ccc(Nc2ncc(C(F)(F)F)c(O[C@@H]3CCOC[C@@H]3O)n2)cc1]

Embodiment I-2. The compound of Embodiment I-1, which is a compound of Formula (II):

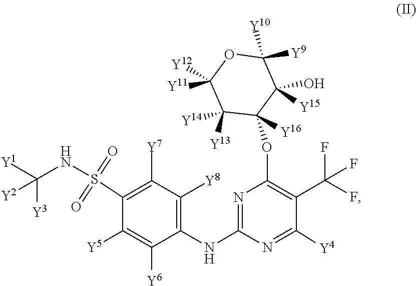

or a stereoisomer, a mixture of stereoisomers, tautomer, or a pharmaceutically acceptable salt thereof, wherein at least one of Y¹, Y², Y³, Y⁴, Y⁵, Y⁶, Y⁷, Y⁸, Y⁹, Y¹⁰, Y¹¹, Y¹², Y¹³, Y¹⁴, Y¹⁵ and Y¹⁶ is a hydrogen that is isotopically enriched with deuterium, and the others of Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, and Y¹⁶ are non-enriched hydrogen atoms.

Embodiment I-3. The compound of Embodiment I-1 or 2, wherein Y¹, Y², and Y³ are all H.

Embodiment I-4. The compound of Embodiment I-1 or 2, wherein Y¹ is D, and Y² and Y³ are H.

Embodiment I-5. The compound of Embodiment I-1 or 2, wherein Y¹ and Y² are D, and Y³ is H.

Embodiment I-6. The compound of Embodiment I-1 or 2, wherein Y¹, Y², and Y³ are all D.

Embodiment I-7. The compound of Embodiment I-2, which is a compound of Formula (III):

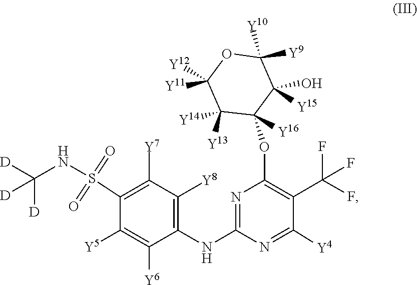

or a stereoisomer, a mixture of stereoisomers, tautomer, or a pharmaceutically acceptable salt thereof, wherein at least one of Y⁴, Y⁵, Y⁶, Y⁷, Y⁸, Y⁹, Y¹⁰, Y¹¹, Y¹², Y¹³, Y¹⁴, Y¹⁵, and Y¹⁶ is a hydrogen that is isotopically enriched with deuterium, and the others of Y⁴, Y⁵, Y⁶, Y⁷, Y⁸, Y⁹, Y¹⁰, Y¹¹, Y¹², Y¹³, Y¹⁴, Y¹⁵, and Y¹⁶ are non-enriched hydrogen atoms.

Embodiment I-8. The compound of any one of Embodiments I-1 to 7, wherein one of Y⁴, Y⁵, Y⁶, Y⁷, Y⁸, Y⁹, Y¹⁰, Y¹¹, Y¹², Y¹³, Y¹⁴, Y¹⁵, and Y¹⁶ is a hydrogen that is isotopically enriched with deuterium, and the others of Y⁴, Y⁵, Y⁶, Y⁷, Y⁸, Y⁹, Y¹⁰, Y¹¹, Y¹², Y¹³, Y¹⁴, Y¹⁵ and Y¹⁶ are non-enriched hydrogen atoms.

Embodiment I-9. The compound of any one of Embodiments I-1 to 7, wherein two of Y⁴, Y⁵, Y⁶, Y⁷, Y⁸, Y⁹, Y¹⁰, Y¹¹, Y¹², Y¹³, Y¹⁴, Y¹⁵, and Y¹⁶ are hydrogen atoms that are isotopically enriched with deuterium, and the others of Y⁴, y, Y⁶, Y⁷, Y⁸, Y⁹, Y¹⁰, Y¹¹, Y¹², Y¹³, Y¹⁴, Y¹⁵, and Y¹⁶ are non-enriched hydrogen atoms.

Embodiment I-10. The compound of any one of Embodiments I-1 to 7, wherein three of Y⁴, Y⁵, Y⁶, Y⁷, Y⁸, Y⁹, Y¹⁰, Y¹¹, Y¹², Y¹³, Y¹⁴, Y¹⁵, and Y¹⁶ are hydrogen atoms that are isotopically enriched with deuterium, and the others of Y⁴, Y⁵, Y⁶, Y⁷, Y⁸, Y⁹, Y¹⁰, Y¹¹, Y¹², Y¹³, Y¹⁴, Y¹⁵, and Y¹⁶ are non-enriched hydrogen atoms.

Embodiment I-11. The compound of any one of Embodiments I-1 to 7, wherein four, five, six, seven, eight, nine, ten, eleven, twelve, or thirteen of Y⁴, Y⁵, Y⁶, Y⁷, Y⁸, Y⁹, Y¹⁰, Y¹¹, Y¹², Y¹³, Y¹⁴, Y¹⁵, and Y¹⁶ are hydrogen atoms that are isotopically enriched with deuterium, and the others of Y⁴, Y⁵, Y⁶, Y⁷, Y⁸, Y⁹, Y¹⁰, Y¹¹, Y¹², Y¹³, Y¹⁴, Y¹⁵, and Y¹⁶ are non-enriched hydrogen atoms.

Embodiment I-12. The compound of any one of Embodiments I-1 to 11, wherein Y⁴ is D.

Embodiment I-13. The compound of any one of Embodiments I-1 to 12, wherein Y¹, Y², Y³ and Y⁴ are all D.

Embodiment I-14. The compound of Embodiment I-1, which is a compound of Formula (III-A), (III-B), (III-C), (III-D), (III-E), (III-F), (III-G), or (III-H).

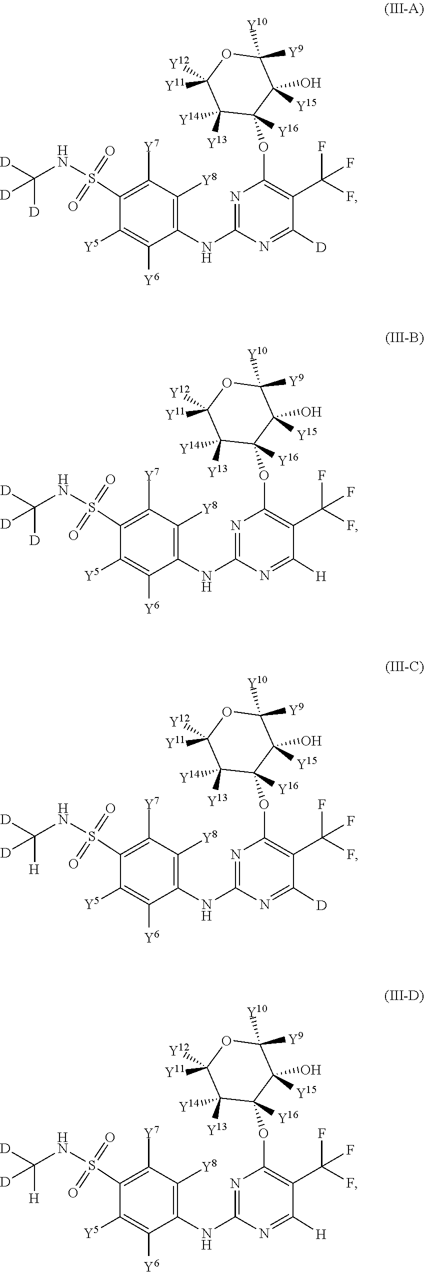

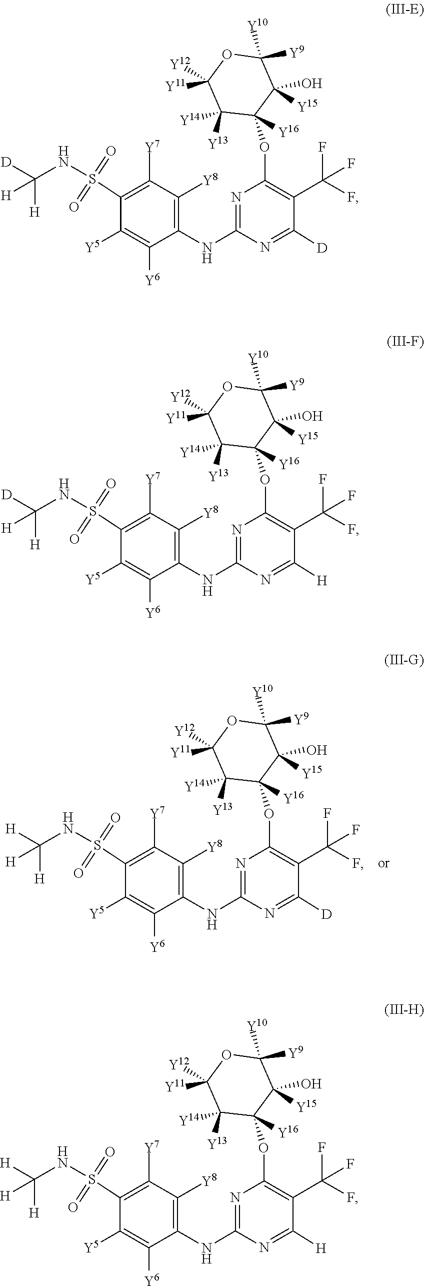

or a stereoisomer, a mixture of stereoisomers, tautomer, or a pharmaceutically acceptable salt thereof.

Embodiment I-15. The compound of Embodiment I-1, which is a compound of Formula (IV):

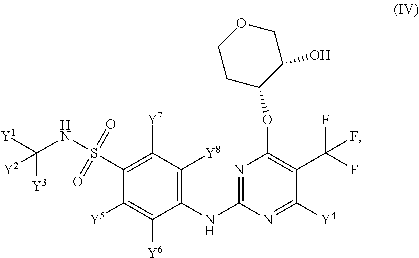

or a stereoisomer, a mixture of stereoisomers, tautomer, or a pharmaceutically acceptable salt thereof, wherein at least one of Y¹, Y², Y³, Y⁴, Y⁵, Y⁶, Y⁷, and Y⁸ is a hydrogen that is isotopically enriched with deuterium, and the others of Y¹, Y², Y², Y³, Y⁴, Y⁵, Y⁶, Y⁷, and Y⁸ are non-enriched hydrogen atoms.

Embodiment I-16. The compound of Embodiment I-1, which is a compound of Formula (V):

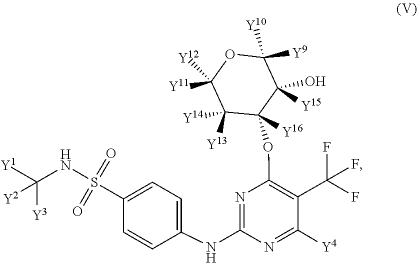

or a stereoisomer, a mixture of stereoisomers, tautomer, or a pharmaceutically acceptable salt thereof, wherein at least one of Y¹, Y², Y³, Y⁴, Y⁹, Y¹⁰, Y¹¹, Y¹², Y¹³, Y¹⁴, Y¹⁵, and Y¹⁶ is a hydrogen that is isotopically enriched with deuterium, and the others of Y¹, Y², Y³, Y⁴, Y⁹, Y¹⁰, Y¹¹, Y¹², Y¹³, Y¹⁴, Y¹⁵, and Y¹⁶ are non-enriched hydrogen atoms.

Embodiment I-17. The compound of Embodiment I-1, which is a compound of Formula (VI):

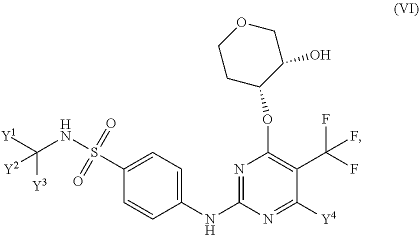

or a stereoisomer, a mixture of stereoisomers, tautomer, or a pharmaceutically acceptable salt thereof, wherein at least one of Y¹, Y², Y³, and Y⁴ is a hydrogen that is isotopically enriched with deuterium, and the others of Y¹, Y², Y³, and Y⁴ are non-enriched hydrogen atoms.

Embodiment I-18. The compound of Embodiment I-1, which is selected from any one of the compounds in Tables 1 to 6, or a stereoisomer, a mixture of stereoisomers, tautomer, or a pharmaceutically acceptable salt thereof.

Embodiment I-19. The compound of Embodiment I-1, which is:

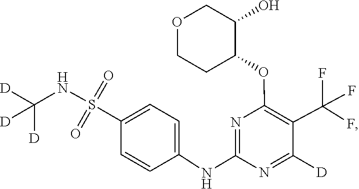 [SMILES: [2H]c1nc(Nc2ccc(S(=O)(=O)NC([2H])([2H])[2H])cc2)nc(O[C@@H]2CCOC[C@@H]2O)c1C(F)(F)F]

or a pharmaceutically acceptable salt thereof.

Embodiment I-20. A pharmaceutical composition comprising a compound of any one of Embodiments I-1 to 19, and a pharmaceutically acceptable excipient.

Embodiment I-21. A method of treating cancer in a subject having cancer, wherein the method comprises administering to the subject a therapeutically effective amount of the compound of any one of Embodiments I-1 to 19 or the pharmaceutical composition of Embodiment I-20.

Embodiment I-22. The method of Embodiment I-21, wherein the cancer is breast cancer, ovarian cancer, bladder cancer, uterine cancer, prostate cancer, lung cancer (including NSCLC, SCLC, squamous cell carcinoma or adenocarcinoma), esophageal cancer, head and neck cancer, colorectal cancer, kidney cancer (including RCC), liver cancer (including HCC), pancreatic cancer, stomach (e.g., gastric) cancer and/or thyroid cancer.

Embodiment I-23. The method of Embodiment I-21, wherein the cancer is breast cancer ER-positive/HR-positive, HER2-negative breast cancer; ER-positive/HR-positive, HER2-positive breast cancer; triple negative breast cancer (TNBC); or inflammatory breast cancer.

Embodiment I-24. The method of Embodiment I-21, wherein the cancer is breast cancer is endocrine resistant breast cancer, trastuzumab resistant breast cancer, or breast cancer demonstrating primary or acquired resistance to CDK4/CDK6 inhibition.

Embodiment I-25. The method of Embodiment I-21, wherein the cancer is advanced or metastatic breast cancer.

Embodiment I-26. The method of Embodiment I-21, wherein the cancer is ovarian cancer.

Embodiment I-27. The method of any one of Embodiments I-21 to 26, wherein the cancer is characterized by an amplification or overexpression of cyclin E1 and/or cyclin E2.

Embodiment I-28. Use of the compound of any one of Embodiments I-1 to 19 or the pharmaceutical composition of Embodiment I-20 for the treatment of cancer.

Embodiment I-29. The compound of any one of Embodiments I-1 to 19 or the pharmaceutical composition of Embodiment I-20 for use in treating cancer.

Embodiment I-30. The compound of any one of Embodiments I-1 to 19 or the pharmaceutical composition of Embodiment I-20 for use as a medicament.

Embodiment I-31. Use of the compound of any one of Embodiments I-1 to 19 or the pharmaceutical composition of Embodiment I-20 for the manufacture of a medicament for the treatment of cancer.

### EXAMPLES

The chemical entities described herein can be synthesized according to one or more illustrative schemes herein and/or techniques well known in the art.

### Example 1: Synthesis of Deuterated Intermediates

(Cis)-tetrahydro-2H-pyran-2,2,6,6-d₄-3,4-diol (Intermediate 1) can be synthesized as a racemic mixture from a known compound S-1 described in U.S. Pat. No. 9,242,996B2 using the procedure described in WO2022111621A1, the entirely of each of which is incorporated herein by reference.

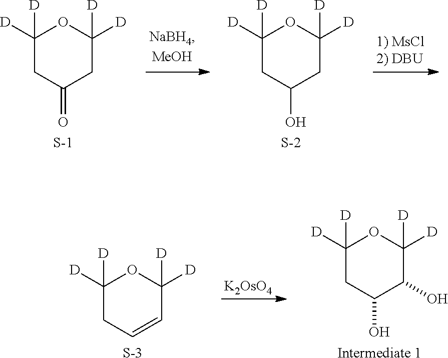

(Cis)-tetrahydro-2H-pyran-2,2,3,4,6,6-d₆-3,4-diol (Intermediate 2). S-4 can be synthesized similarly from S-1 (U.S. Pat. No. 9,242,996B2) using the procedure described in Eur. J. Org. Chem., 2012, 33, 6586, which is incorporated herein by reference. S-5 can be synthesized similarly from S-4 using the procedure described in Helvetica Chimica Acta, 2004, 87, 227-239. Intermediate 2 as a racemic mixture can be synthesized similarly from S-5 using the procedure described in WO2022111621A1.

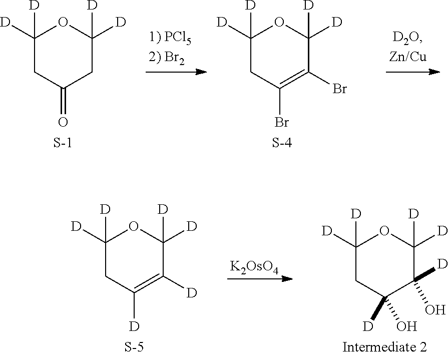

(Cis)-tetrahydro-2H-pyran-3,4-d₂-3,4-diol (Intermediate 3) as a racemic mixture can be synthesized using the analogous method described above for Intermediate 2 but using commercially available S-6 as the starting material.

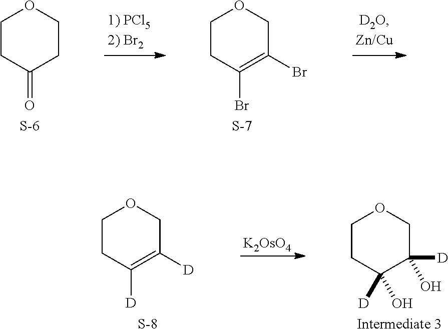

(3S,4R)-tetrahydro-2H-pyran-4-d-3,4-diol (Intermediate 4) and (3S,4R)-tetrahydro-2H-pyran-3-d-3,4-diol (Intermediate 5) can be synthesized from S-9 described in WO2023093769 via the routes designed below:

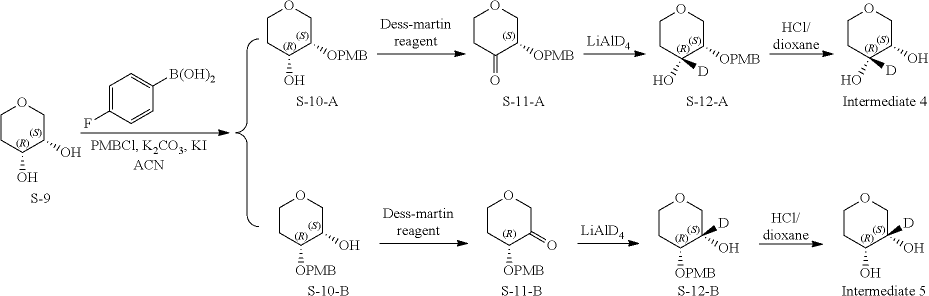

4-amino-N-(methyl-d₃)benzene-2,3,5,6-d₄-sulfonamide (Intermediate 6). S-14 can be synthesized from commercially available S-13 using the procedure described in CN110028457, which is incorporated herein by reference. S-14 can be readily converted into sulfonamide S-15, which can be further hydrolyzed into Intermediate 6 using the procedure described in Eur. J Med. Chem. 2014, 71, 1.

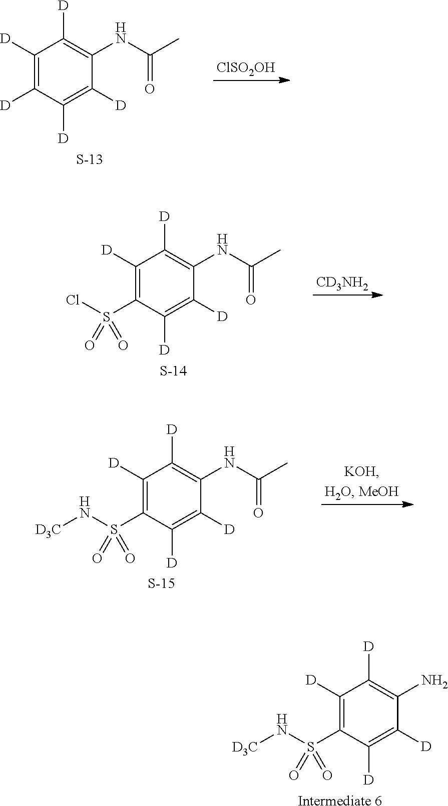

### Example 2: Synthesis of Deuterated Compounds

### Synthesis of 4-((4-(((3S,4R)-3-hydroxytetrahydro-2H-pyran-4-yl)oxy)-5-(trifluoromethyl)pyrimidin-2-yl-6-d)amino)-N-(methyl-d₃)benzenesulfonamide (Compound I-H)

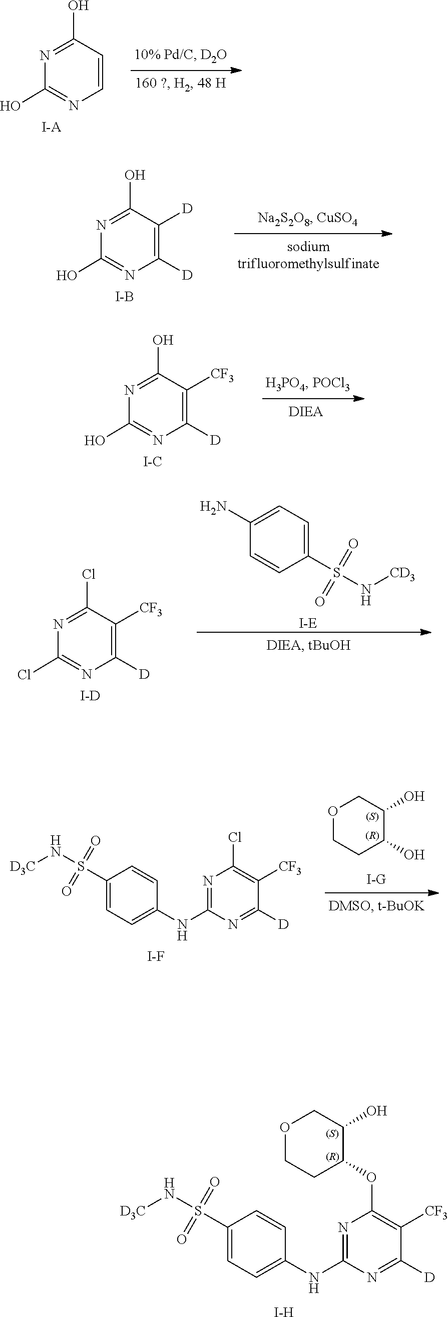

Preparation of Compound I-B: Pyrimidine-2,4-diol (I-A, 2.5 g, 22.32 mmol) and 10% Pd/C (250 mg, 55 wt % of the substrate) in D₂O (50 mL) were stirred at 160° C. in an autoclave under H₂ (0.8 MPa) atmosphere for 48 h. After cooling to room temperature, the reaction mixture was filtered, and the filtered catalyst was washed with boiling water (150 mL). The combined filtrates were concentrated in vacuo to give the deuterated pyrimidine-2,4-diol as a white powder (I-B, 2.5 g, 21.93 mmol, 98%). The deuterium content (%) was determined by ¹H NMR using 3-trimethylsilyl-1-propanesulfonic acid sodium salt (DSS) as the internal standard and confirmed by mass spectroscopy. LC-MS (ESI): m/z 115.1 [M+H]⁺.

Preparation of Compound I-C: A mixture of pyrimidine-d₂-2,4-diol (I-B, 2.5 g, 21.93 mmol) and sodium trifluoromethanesulfinate (5.13 g, 32.87 mmol) in 15 ml of water was stirred with temperature raised to and maintained at 60~65° C. Disodium dioxidanedisulfonate (10.43 g, 43.82 mmol) and cupric sulfate (1.05 g, 6.57 mmol) were added in 10 batches. After 1 hour of stirring, the mixture was extracted with ethyl acetate (3×5 mL). The organic layers were combined, dried over sodium sulfate, concentrated under reduced pressure, and the residue separated using silica gel column chromatography (ethyl acetate/petroleum ether=20-80%) to afford 5-(trifluoromethyl)pyrimidine-6-d-2,4-diol (I-C, 1.9 g, 10.50 mmol, 47.91%) as a white solid. LC-MS (ESI): m z 182.2 [M+H]⁺.

Preparation of Compound I-D: To a mixture of 5-(trifluoromethyl)pyrimidine-6-d-2,4-diol (I-C, 1.9 g, 10.50 mmol) and phosphorus oxychloride (8.05 g, 52.49 mmol) in a 100 mL flask under a nitrogen atmosphere, concentrated phosphonic acid (102.86 mg, 1.05 mmol) was added slowly. Diisopropylethylamine (1.70 mL, 10.50 mmol) was then added dropwise over 2 minutes. The orange solution was heated to and maintained at 100° C. After 20 hours, the reaction mixture was cooled to 40° C. HCl (3N, 15 mL) and diethyl ether (10 mL) were added with the temperature maintained between 10 to 15° C. The layers were separated, and the aqueous layer was extracted once with ethyl acetate (50 mL). The organic extract and the organic layer of the reaction mixture were combined and washed with water until the washes were neutral (5×15 mL washes), dried with sodium sulfate and concentrated. The oily residue was further purified with silica gel column chromatography to afford 2,4-dichloro-5-(trifluoromethyl)pyrimidine-6-d (I-D, 1.1 g, 5.07 mmol, 48.3%) as a colorless oil.

Preparation of Compound I-F: To a mixture of 4-amino-N-(methyl-d₃)benzenesulfonamide (I-E, 305.24 mg, 1.61 mmol) and 2,4-dichloro-5-(trifluoromethyl)pyrimidine-6-d (I-D, 350 mg, 1.61 mmol). in tertiary butanol (10 mL) was added diisopropylethylamine (625.44 mg, 4.84 mmol). The reaction mixture was stirred at 80° C. under N₂ overnight. After completion, the mixture was concentrated under reduced pressure. The residue was diluted with water (20 mL) and extracted with ethyl acetate (10 mL×3). The combined organic layers were washed with brine (20 mL), dried over sodium sulfate, and concentrated under reduced pressure. The residue was subjected to preparative HPLC to afford 4-((4-chloro-5-(trifluoromethyl)pyrimidin-2-yl-6-d)amino)-N-(methyl-d₃)benzenesulfonamide (I-F, 228 mg, 0.62 mmol, 38.1%) as white solid. LC-MS (ESI): m z 371.1 [M+H]⁺.

Preparation of Compound I-H: To a solution of 4-{[6-chloro-4-deuterio-5-(trifluoromethyl)pyrimidin-2-yl]amino}-N-(trideuteriomethyl)benzenesulfonamide (I-F, 200 mg, 0.54 mmol) in dimethylsulfoxide (2 mL) were added (3S,4R)-tetrahydropyran-3,4-diol (I-G, 63.72 mg, 0.54 mmol) and potassium tert-butoxide (242.11 mg, 2.16 mmol). The reaction mixture was stirred at 90° C. for 2 hours. After completion, the reaction mixture was diluted with water (10 mL) and extracted with ethyl acetate (2×5 mL). The combined organic layers were washed with brine (10 mL), dried over sodium sulfate and concentrated under reduced pressure to yield a residue, which was subjected to silica gel column chromatography followed by chiral HPLC separation to give 4-((4-(((3S,4R)-3-hydroxytetrahydro-2H-pyran-4-yl)oxy)-5-(trifluoromethyl)pyrimidin-2-yl-6-d)amino)-N-(methyl-d₃)benzenesulfonamide (65 mg, 0.14 mmol, 26.63%). HPLC retention time: 0.93 min. LC-MS (ESI): m/z 453.2 [M+H]⁺. ¹H NMR (500 MHz, DMSO-d₆) δ 10.50 (s, 1H), 8.60 (s, 0.06H), 7.93 (d, J=8.4 Hz, 2H), 7.74 (d, J=9.0 Hz, 2H), 7.27 (s, 1H), 5.60 (s, 1H), 5.05 (d, J=4.9 Hz, 1H), 3.90 (s, 1H), 3.71-3.50 (m, 4H), 2.09-1.84 (m, 2H).

Analytical method: Instrument: SHIMADZU-20AD-XR; Column: Cellulose SB, 4.6*50 mm, 3.0 μm; Mobile phase: A for Hex (0.1% DEA) and B for EtOH; Gradient: B 30%; Flow rate: 1.67 mL/min; High pressure: 110 bar; Column temperature: 25° C.; Wavelength: 280 nm

Preparative separation method: Instrument: GILSON-LC07; Column: Cellulose SB, 20*250 mm, 5 μm; Mobile phase: A for Hex (10 Mm NH₃) and B for EtOH; Gradient: B 30%; Flow rate: 20 mL/min; High pressure: 58 bar; Column temperature: 25° C.; Wavelength: 297 nm/310 nm; Cycle time: ~12 min. Sample preparation: the sample was prepared by dissolving Compound I-H in about 6 mL MeOH/DCM; Injection: 0.5 ml per injection. Work up: after separation, the fractions were dried off via rotary evaporator at bath temperature 30° C. and by freeze-drying.

### Synthesis of 4-((4-(((3S,4R)-3-hydroxytetrahydro-2H-pyran-4-yl-2,2,6,6-d₄)oxy)-5-(trifluoromethyl)pyrimidin-2-yl)amino)-N-(methyl-d₃)benzenesulfonamide (Compound P1)

Compound P1 can be synthesized from Intermediate 1 (racemic) and S-16 described in WO2022111621A1, which is incorporated herein by reference. S-17 is prepared as a racemic mixture, and can be purified with chiral HPLC to obtain P1 and SPI.

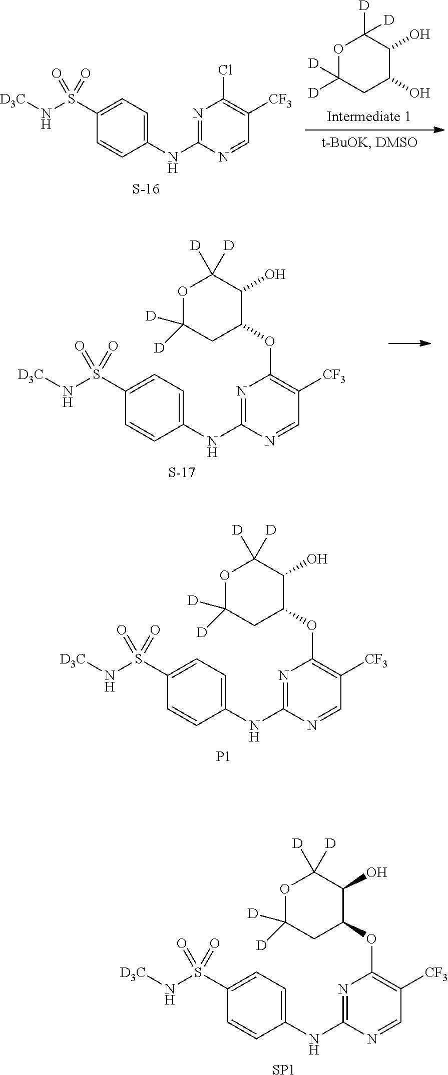

This procedure can be applied similarly to other diol intermediates (e.g. Intermediate 2, Intermediate 3, Intermediate 4 and Intermediate 5) to synthesize corresponding deuterated compounds, such as compounds P2, P3, P4, and P5 below:

[symbol]

### Synthesis of 4-((4-(((3S,4R)-3-hydroxytetrahydro-2H-pyran-4-yl)oxy)-5-(trifluoromethyl)pyrimidin-2-yl)amino)-N-(methyl-d)benzene-2,3,5,6-d₄-sulfonamide (Compound P6)

Compound P6 can be synthesized using the procedure reported in WO2022111621A1 using Intermediate 6.

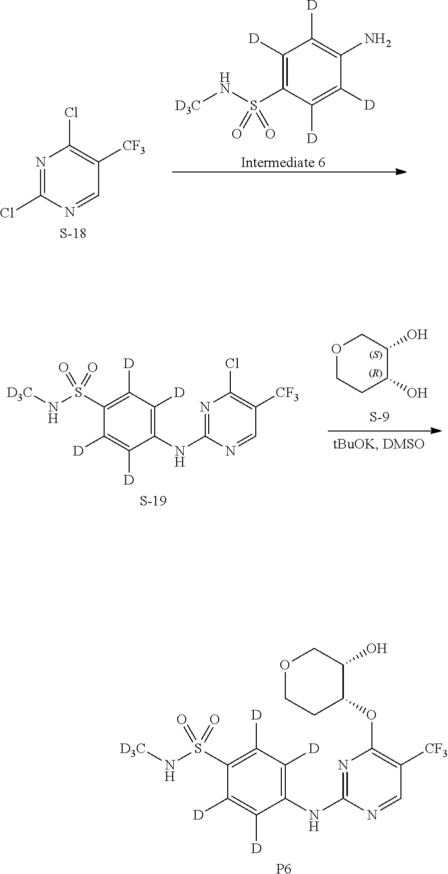

### Example 3: Biological Activity

CDK2/CyclinE1 kinase inhibitory activity (IC50): 5 pd of various dilutions of test compounds in 1× kinase buffer (50 mM HEPES pH 7.5, 10 mM MgCl2, 2 mM DTT and 0.01% Brij-35) were mixed with 10 μL of CDK2/CyclinE1 (Cama, 04-165 #, final concentration 3 nM in 1×Kinase buffer) in 384 plates and incubated at room temperature for 10 min. To initiate each reaction, 10 μL of peptide solution containing fluorescently-labeled-peptide 18(5-FAM-QSPKKG-CONH2) (GL, 114202 #, final concentration 3000 nM) and ATP (final concentration 77 μM) in 1×Kinase buffer was added to each of the wells containing test compound and CDK2/CyclinE1 mixture. The reaction was then allowed to proceed at 28° C. for 30 min and terminated by the addition of 25 μL stop buffer (100 mM HEPES pH 7.5, 50 mM EDTA, 0.2% Coating Reagent #3 (Perkin Elmer, 760050 #) and 0.015% Brij-35).

Following the kinase reaction, Caliper EZ reader II (Downstream voltages: −500V, Upstream voltages: −2250V, Base pressure −0.5 PSI, Screen pressure −1.2 PSI) was used to separate the phosphorylated (product) and the unphosphorylated (substrate) fluorescently-labeled peptide 18 based on their different mobility. Both substrate and product were measured and the ratio of these values were used to generate % conversion by Caliper EZ reader II. These conversion values were then transformed into % inhibition of kinase activity using the formula: % Inhibition=[(MA−X)/(MA−MI)]×100% where MA=conversion value of DMSO only controls, MI=conversion value of no enzyme controls and X=conversion value at any given compound dose. IC50 values were then calculated by plotting dose-response curves and then using the XLfit application in Excel software.

CDK1/CyclinB kinase inhibitory activity (IC50): 5 μl of various dilutions of test compound in 1×kinase buffer (50 mM HEPES pH 7.5, 10 mM MgCl2, 2 mM DTT and 0.01% Brij-35) was mixed with 10 μL of CDK1/CyclinB (Millipore, 14-450M #, final concentration 3 nM in 1×Kinase buffer) in 384 plates and incubated at room temperature for 10 min. To initiate each reaction, 10 μL of peptide solution containing fluorescently-labeled-peptide 18(5-FAM-QSPKKG-CONH2) (GL, 114202 #, final concentration 3000 nM) and ATP (final concentration 20 μM) in 1×Kinase buffer was added to each of the wells containing test compound and CDK1/CyclinB mixture. The reaction is then allowed to proceed at 28° C. for 30 min and terminated by the addition of 25 μL stop buffer (100 mM HEPES pH 7.5, 50 mM EDTA, 0.2% Coating Reagent #3 (Perkin Elmer, 760050 #) and 0.015% Brij-35).

Following the kinase reaction, Caliper EZ reader II (Downstream voltages: −500V, Upstream voltages: −2250V, Base pressure −0.5 PSI, Screen pressure −1.2 PSI) was used to separate the phosphorylated (product) and the unphosphorylated (substrate) fluorescently-labeled peptide 18 based on their different mobility. Both substrate and product were measured and the ratio of these values were used to generate % conversion by Caliper EZ reader II. These conversion values were then transformed into % inhibition of kinase activity using the formula: % Inhibition=[(MA−X)/(MA−MI)]×100% where MA=conversion value of DMSO only controls, MI=conversion value of no enzyme controls and X=conversion value at any given compound dose. IC50 values were then calculated by plotting dose-response curves and then using the XLfit application in Excel software.

CDK4/CyclinD1 kinase inhibitory activity (IC50): 5 μl of various dilutions of test compound in 1× kinase buffer (20 mM HEPES, pH 7.5, 10 mM MgCl2, 2 mM DTT and 0.01% Triton X-100) was mixed with 10 μL of either CDK4/Cyclin D1 (ProQinase, 0142-0143-1 #, final concentration 20 nM in 1× Kinase buffer) or CDK4/CyclinD3 (Carna, 04-105 #, final concentration 10 nM in 1×Kinase buffer) in 384 plates and incubated at room temperature for 10 min. To initiate each reaction, 10 μL of peptide solution containing fluorescently-labeled-peptide 8(5-FAM-IPTSPITTTYFFFKKK-COOH, GL, 112396 #, final concentration 3000 nM) and ATP (final concentration 672 uM for CDK4/CyclinD1 or 280 μM for CDK4/Cyclin D3) in 1×Kinase buffer was added to each of the wells containing test compound and CDK4/CyclinD3 mixture. The reaction is then allowed to proceed at 28° C. for 30 min and terminated by the addition of 25 μL stop buffer (100 mM HEPES pH 7.5, 50 mM EDTA, 0.2% Coating Reagent #3 (Perkin Elmer, 760050 #) and 0.015% Brij-35).

Following the kinase reaction, Caliper EZ reader II (Downstream voltages: −500V, Upstream voltages: −2250V, Base pressure −0.5 PSI, Screen pressure −1.2 PSI) was used to separate the phosphorylated (product) and the unphosphorylated (substrate) fluorescently-labeled peptide 8 based on their different mobility. Both substrate and product were measured and the ratio of these values were used to generate % conversion by Caliper EZ reader II. These conversion values were then transformed into % inhibition of kinase activity using the formula: % Inhibition=[(MA−X)/(MA−MI)]×100% where MA=conversion value of DMSO only controls, MI=conversion value of no enzyme controls and X=conversion value at any given compound dose. IC50 values were then calculated by plotting dose-response curves and then using the XLfit application in Excel software.

CDK6/CyclinD1 kinase inhibitory activity (IC50): 5 μl of various dilutions of test compound in 1× kinase buffer (50 mM HEPES pH 7.5, 10 mM MgCl2, 2 mM DTT and 0.01% Brij-35) was mixed with 10 μL of CDK6/CyclinD1 (Carna, 04-114 #, final concentration 7.5 nM in 1× Kinase buffer) or CDK6/Cyclin D3 (Carna, 04-107 #, final concentration 15 nM in 1× Kinase buffer) in 384 plates and incubated at room temperature for 10 min. To initiate each reaction, 10 μL of peptide solution containing fluorescently-labeled-peptide 8(5-FAM-IPTSPITTTYFFFKKK-COOH, GL, 112396 #, final concentration 3000 nM) and ATP (final concentration 230 μM for CDK6/CyclinD1 or 800 uM for CDK6/CyclinD3) in 1× Kinase buffer was added to each of the wells containing test compound and CDK6/CyclinD1 or CDK6/Cyclin D3 mixture. The reaction is then allowed to proceed at 28° C. for 30 min and terminated by the addition of 25 μL stop buffer (100 mM HEPES pH 7.5, 50 mM EDTA, 0.2% Coating Reagent #3 (Perkin Elmer, 760050 #) and 0.015% Brij-35).

Following the kinase reaction, Caliper EZ reader II (Downstream voltages: −500V, Upstream voltages: −2250V, Base pressure −0.5 PSI, Screen pressure −1.2 PSI) was used to separate the phosphorylated (product) and the unphosphorylated (substrate) fluorescently-labeled peptide 8 based on their different mobility. Both substrate and product were measured and the ratio of these values were used to generate % conversion by Caliper EZ reader II. These conversion values were then transformed into % inhibition of kinase activity using the formula: % Inhibition=[(MA−X)/(MA−MI)]×100% where MA=conversion value of DMSO only controls, MI=conversion value of no enzyme controls and X=conversion value at any given compound dose. IC50 values were then calculated by plotting dose-response curves and then using the XLfit application in Excel software.

CDK7/CyclinH/MAT1 kinase inhibitory activity (IC50): 5 μl of various dilutions of test compound in 1× kinase buffer (20 mM HEPES, pH 7.5, 10 mM MgCl2, 2 mM DTT and 0.01% Triton X-100) was mixed with 10 μL of CDK7/CyclinH/MAT1 (Millipore, 14-476M #, final concentration 12.5 nM in 1× Kinase buffer) in 384 plates and incubated at room temperature for 10 min. To initiate each reaction, 10 μL of peptide solution containing fluorescently-labeled-peptide CTD3 (5-FAM-ACSYSPTSPSYSPTSPSYSPTSPSKK, GL, SY346885 #, final concentration 3000 nM) and ATP (final concentration 70 μM) in 1× Kinase buffer was added to each of the wells containing test compound and CDK7/CyclinH/MAT1 mixture. The reaction is then allowed to proceed at 28° C. for 30 min and terminated by the addition of 25 μL stop buffer (100 mM HEPES pH 7.5, 50 mM EDTA, 0.2% Coating Reagent #3 (Perkin Elmer, 760050 #) and 0.015% Brij-35).

Following the kinase reaction, Caliper EZ reader II (Downstream voltages: −500V, Upstream voltages: −2250V, Base pressure −0.5 PSI, Screen pressure −1.2 PSI) was used to separate the phosphorylated (product) and the unphosphorylated (substrate) fluorescently-labeled peptide CTD3 based on their different mobility. Both substrate and product were measured and the ratio of these values were used to generate % conversion by Caliper EZ reader II. These conversion values were then transformed into % inhibition of kinase activity using the formula: % Inhibition=[(MA−X)/(MA−MI)]×100% where MA=conversion value of DMSO only controls, MI=conversion value of no enzyme controls and X=conversion value at any given compound dose. IC50 values were then calculated by plotting dose-response curves and then using the XLfit application in Excel software.

CDK9/CyclinT1 kinase inhibitory activity (IC50): 5 μl of various dilutions of test compound in 1× kinase buffer (20 mM HEPES, pH 7.5, 10 mM MgCl2, 2 mM DTT and 0.01% Triton X-100) was mixed with 10 μL of CDK9/CyclinT1 (Millipore, 14-685M #, final concentration 12.5 nM in 1× Kinase buffer) in 384 plates and incubated at room temperature for 10 min. To initiate each reaction, 10 μL of peptide solution containing fluorescently-labeled-peptide CTD3 (5-FAM-ACSYSPTSPSYSPTSPSYSPTSPSKK, GL, SY346885 #, final concentration 3000 nM) and ATP (final concentration 10 μM) in 1× Kinase buffer was added to each of the wells containing test compound and CDK9/CyclinT1 mixture. The reaction is then allowed to proceed at 28° C. for 30 min and terminated by the addition of 25 μL stop buffer (100 mM HEPES pH 7.5, 50 mM EDTA, 0.2% Coating Reagent #3 (Perkin Elmer, 760050 #) and 0.015% Brij-35).

Following the kinase reaction, Caliper EZ reader II (Downstream voltages: −500V, Upstream voltages: −2250V, Base pressure −0.5 PSI, Screen pressure −1.2 PSI) was used to separate the phosphorylated (product) and the unphosphorylated (substrate) fluorescently-labeled peptide CTD3 based on their different mobility. Both substrate and product were measured and the ratio of these values were used to generate % conversion by Caliper EZ reader II. These conversion values were then transformed into % inhibition of kinase activity using the formula: % Inhibition=[(MA−X)/(MA−MI)]×100% where MA=conversion value of DMSO only controls, MI=conversion value of no enzyme controls and X=conversion value at any given compound dose. IC50 values were then calculated by plotting dose-response curves and then using the XLfit application in Excel software.

Biological activity data for representative deuterated compounds of the present application are provided in Table 7 below. Exemplary results are presented as calculated IC₅₀ values. In Table 7, “A” represents a calculated IC₅₀ value of less than 10 nM; “B” represents a calculated IC₅₀ value of greater than or equal to 10 nM and less than 100 nM; “C” represents a calculated IC₅₀ value of greater than or equal to 100 nM and less than 1 μM; and “D” represents a calculated IC₅₀ value of 1 μM or greater.

TABLE 7
Selected in vitro IC₅₀(nM) data on different CDKs

| | CDK1/ | CDK2/ | CDK4/ | CDK6/ | CDK7/ | CDK9/ |
|---|---|---|---|---|---|---|
| | Cyclin | Cyclin | Cyclin | Cyclin | Cyclin | Cyclin |
| Compound structure | B1 | E1 | D1 | D1 | H/MAT1 | T1 |
| 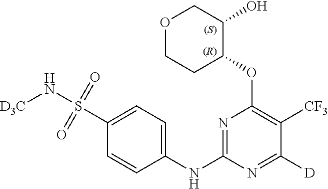 | B | A | C | C | C | C |

The present invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims. Various publications, patents and patent applications are cited herein, the applications of which are incorporated by reference in their entireties.

1-31: (canceled)

32. A method of producing 4-amino-N-(methyl-d₃)benzene-2,3,5,6-d₄-sulfonamide, the method comprising hydrolyzing N-(4-(N-(methyl-d₃)sulfamoyl)phenyl-2,3,5,6-d₄)acetamide.

33. The method of claim 32, further comprising reacting 4-acetamidobenzenesulfonyl chloride-2,3,5,6-d₄ with methan-d₃-amine to produce the N-(4-(N-(methyl-d₃)sulfamoyl)phenyl-2,3,5,6-d₄)acetamide.

34. The method of claim 33, further comprising reacting N-(phenyl-d₅)acetamide with sulfurochloridic acid to produce the 4-acetamidobenzenesulfonyl chloride-2,3,5,6-d₄.

35. A method of producing 2,4-dichloro-5-(trifluoromethyl)pyrimidine-6-d, the method comprising reacting 5-(trifluoromethyl)pyrimidine-6-d-2,4-diol with phosphoryl chloride.

36. The method of claim 35, further comprising reacting pyrimidine-d₂-2,4-diol with sodium trifluoromethylsulfinate to produce the 5-(trifluoromethyl)pyrimidine-6-d-2,4-diol.

37. The method of claim 36, further comprising reacting pyrimidine-2,4-diol with D₂O under an H₂ atmosphere and in the presence of a Pd/C catalyst to product the pyrimidine-d₂-2,4-diol.

38. A compound selected from:

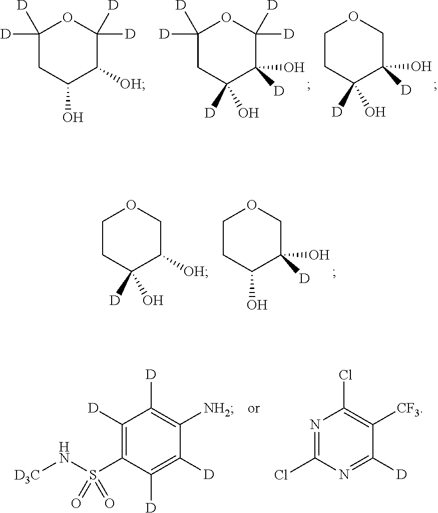

39. The compound of claim 38, wherein the compound is:

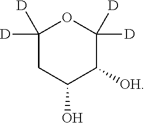 [SMILES: [2H]C1([2H])C[C@@H](O)[C@@H](O)C([2H])([2H])O1]

40. The compound of claim 38, wherein the compound is:

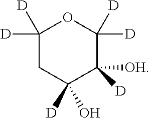 [SMILES: [2H]C1([2H])C[C@@]([2H])(O)[C@@]([2H])(O)C([2H])([2H])O1]

41. The compound of claim 38, wherein the compound is:

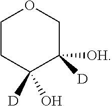

42. The compound of claim 38, wherein the compound is:

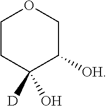

43. The compound of claim 38, wherein the compound is:

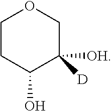

44. The compound of claim 38, wherein the compound is:

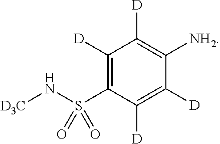 [SMILES: [2H]c1c([2H])c(S(=O)(=O)NC([2H])([2H])[2H])c([2H])c([2H])c1N]

45. The compound of claim 38, wherein the compound is: 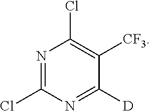 [SMILES: [2H]c1nc(Cl)nc(Cl)c1C(F)(F)F]
